SEQUENCE LISTING (152 sequences)

SEQ ID NO: 1 - protein, 8 aa, Artificial Sequence
  Heavy chain variable region CDR sequence
VYSFTAYT

SEQ ID NO: 2 - protein, 8 aa, Artificial Sequence
  Heavy chain variable region CDR sequence
INPHNGGT

SEQ ID NO: 3 - protein, 14 aa, Artificial Sequence
  Heavy chain variable region CDR sequence
AISRYGSSSFYFDV

SEQ ID NO: 4 - protein, 8 aa, Artificial Sequence
  Heavy chain variable region CDR sequence
GYAFTNYW

SEQ ID NO: 5 - protein, 8 aa, Artificial Sequence
  Heavy chain variable region CDR sequence
FYPRTGNT

SEQ ID NO: 6 - protein, 9 aa, Artificial Sequence
  Heavy chain variable region CDR sequence
ARAGTGFDY

SEQ ID NO: 7 - protein, 8 aa, Artificial Sequence
  Heavy chain variable region CDR sequence
GYRFSSYW

SEQ ID NO: 8 - protein, 8 aa, Artificial Sequence
  Heavy chain variable region CDR sequence
ILPGRGII

SEQ ID NO: 9 - protein, 10 aa, Artificial Sequence
  Heavy chain variable region CDR sequence
ARTDPPYFGV

SEQ ID NO: 10 - protein, 8 aa, Artificial Sequence
  Heavy chain variable region CDR sequence
GYTFSTYW

SEQ ID NO: 11 - protein, 8 aa, Artificial Sequence
  Heavy chain variable region CDR sequence
NLPGRHIT

SEQ ID NO: 12 - protein, 11 aa, Artificial Sequence
  Heavy chain variable region CDR sequence
ARGRGTYYFDY

SEQ ID NO: 13 - protein, 8 aa, Artificial Sequence
  Heavy chain variable region CDR sequence
GYSFTGYT

SEQ ID NO: 14 - protein, 8 aa, Artificial Sequence
  Heavy chain variable region CDR sequence
INPYNGGT

SEQ ID NO: 15 - protein, 14 aa, Artificial Sequence
  Heavy chain variable region CDR sequence
AFSYYGSRGFYFDY

SEQ ID NO: 16 - protein, 8 aa, Artificial Sequence
  Heavy chain variable region CDR sequence
GYSFTGYT

SEQ ID NO: 17 - protein, 8 aa, Artificial Sequence
  Heavy chain variable region CDR sequence
INPYNGGT

SEQ ID NO: 18 - protein, 15 aa, Artificial Sequence
  Heavy chain variable region CDR sequence
ASSSYRNDGNWYFDV

SEQ ID NO: 19 - protein, 8 aa, Artificial Sequence
  Heavy chain variable region CDR sequence
GYSITGYT

SEQ ID NO: 20 - protein, 8 aa, Artificial Sequence
  Heavy chain variable region CDR sequence
VNPYNGGT

SEQ ID NO: 21 - protein, 14 aa, Artificial Sequence
  Heavy chain variable region CDR sequence
AISRYGSESWYFDV

SEQ ID NO: 22 - protein, 8 aa, Artificial Sequence
  Heavy chain variable region CDR sequence
GYTFNIYW

SEQ ID NO: 23 - protein, 8 aa, Artificial Sequence
  Heavy chain variable region CDR sequence
ILPGSGNT

SEQ ID NO: 24 - protein, 11 aa, Artificial Sequence
  Heavy chain variable region CDR sequence
ARTDGRGYFDY

SEQ ID NO: 25 - protein, 8 aa, Artificial Sequence
  Heavy chain variable region CDR sequence
GYTFSSYW

SEQ ID NO: 26 - protein, 8 aa, Artificial Sequence
  Heavy chain variable region CDR sequence
FLPRSGKT

SEQ ID NO: 27 - protein, 10 aa, Artificial Sequence
  Heavy chain variable region CDR sequence
ARTDPPYFGV

SEQ ID NO: 28 - protein, 5 aa, Artificial Sequence
  Light chain variable region CDR sequence
SSISY

SEQ ID NO: 29 - protein, 3 aa, Artificial Sequence
  Light chain variable region CDR sequence
ATS

SEQ ID NO: 30 - protein, 9 aa, Artificial Sequence
  Light chain variable region CDR sequence
QQWSSNPPT

SEQ ID NO: 31 - protein, 6 aa, Artificial Sequence
  Light chain variable region CDR sequence
ENVGGY

SEQ ID NO: 32 - protein, 3 aa, Artificial Sequence
  Light chain variable region CDR sequence
GAS

SEQ ID NO: 33 - protein, 9 aa, Artificial Sequence
  Light chain variable region CDR sequence
GQNYIYPFT

SEQ ID NO: 34 - protein, 9 aa, Artificial Sequence
  Light chain variable region CDR sequence
TGAVTISNY

SEQ ID NO: 35 - protein, 3 aa, Artificial Sequence
  Light chain variable region CDR sequence
GTN

SEQ ID NO: 36 - protein, 9 aa, Artificial Sequence
  Light chain variable region CDR sequence
VLWYSNHWV

SEQ ID NO: 37 - protein, 11 aa, Artificial Sequence
  Light chain variable region CDR sequence
QSLLHSNGITY

SEQ ID NO: 38 - protein, 3 aa, Artificial Sequence
  Light chain variable region CDR sequence
QMS

SEQ ID NO: 39 - protein, 9 aa, Artificial Sequence
  Light chain variable region CDR sequence
AQNLEFPFT

SEQ ID NO: 40 - protein, 5 aa, Artificial Sequence
  Light chain variable region CDR sequence
SSVSY

SEQ ID NO: 41 - protein, 3 aa, Artificial Sequence
  Light chain variable region CDR sequence
LTS

SEQ ID NO: 42 - protein, 9 aa, Artificial Sequence
  Light chain variable region CDR sequence
QQWSSNPPT

SEQ ID NO: 43 - protein, 5 aa, Artificial Sequence
  Light chain variable region CDR sequence
SSVSY

SEQ ID NO: 44 - protein, 3 aa, Artificial Sequence
  Light chain variable region CDR sequence
DTS

SEQ ID NO: 45 - protein, 9 aa, Artificial Sequence
  Light chain variable region CDR sequence
QQWSSNPPT

SEQ ID NO: 46 - protein, 5 aa, Artificial Sequence
  Light chain variable region CDR sequence
SSVSY

SEQ ID NO: 47 - protein, 3 aa, Artificial Sequence
  Light chain variable region CDR sequence
ATS

SEQ ID NO: 48 - protein, 9 aa, Artificial Sequence
  Light chain variable region CDR sequence
QQWSSNPPT

SEQ ID NO: 49 - protein, 10 aa, Artificial Sequence
  Light chain variable region CDR sequence
KSVSSSAYSY

SEQ ID NO: 50 - protein, 3 aa, Artificial Sequence
  Light chain variable region CDR sequence
LAS

SEQ ID NO: 51 - protein, 9 aa, Artificial Sequence
  Light chain variable region CDR sequence
QHSRELPFT

SEQ ID NO: 52 - protein, 5 aa, Artificial Sequence
  Light chain variable region CDR sequence
SNISY

SEQ ID NO: 53 - protein, 3 aa, Artificial Sequence
  Light chain variable region CDR sequence
DTS

SEQ ID NO: 54 - protein, 9 aa, Artificial Sequence
  Light chain variable region CDR sequence
QQWSSVPLT

SEQ ID NO: 55 - protein, 121 aa, Artificial Sequence
  Heavy chain variable region
EVLLQQSGPELVKPGASMKISCKASVYSFTAYTMNWVKQSHGKNLEWIGLINPHNGGTRYNQKFKGKATLTLDKSSSTAYMDLLSLTSEDSAVYYCAISRYGSSSFYFDVWGAGTTVAVSS

SEQ ID NO: 56 - protein, 116 aa, Artificial Sequence
  Heavy chain variable region
QVQLQQSGAELVRPGTSVKISCKASGYAFTNYWLGWMKQRPGHGLEWIGDFYPRTGNTFYNENFKGKVTLTADKSSNTAYMQLSSLTSEDSAVYLCARAGTGFDYWGQGTTLTVSS

SEQ ID NO: 57 - protein, 117 aa, Artificial Sequence
  Heavy chain variable region
QVQLQQSGAELMKPGASVKISCKTTGYRFSSYWIEWVKQRPGHGLEWLGEILPGRGIINYNENFRGKATFTADTSSNTAYVQLSSLTSEDSAVYFCARTDPPYFGVWGAGTTVTVSS

SEQ ID NO: 58 - protein, 118 aa, Artificial Sequence
  Heavy chain variable region
QVQLQQSGAELMKPGASMKISCKATGYTFSTYWIEWVKQRPGHGLEWIGENLPGRHITNYNEKFKGKATFTADTSSNTAYMQLSSLTSEDSAVYYCARGRGTYYFDYWGQGTPLTVSS

SEQ ID NO: 59 - protein, 121 aa, Artificial Sequence
  Heavy chain variable region
EVQLQQSGPELVKPGASMKISCKASGYSFTGYTMNWVKQSHGKNLEWIGLINPYNGGTNYNQKFKGKATLTVDKSSSTAYMELLSLTSEDSAVYYCAFSYYGSRGFYFDYWGQGTTLTVSS

SEQ ID NO: 60 - protein, 122 aa, Artificial Sequence
  Heavy chain variable region
EVQLQQSGPELVKPGASMKISCKASGYSFTGYTMNWVKQSHGKNLEWIGLINPYNGGTRYNQKFKGKATLTVDKSSSTAYMELLSLTSEDSAVYYCASSSYRNDGNWYFDVWGAGTTVTVSS

SEQ ID NO: 61 - protein, 121 aa, Artificial Sequence
  Heavy chain variable region
EVQLQQSGPELVKPGASMKISCKASGYSITGYTMNWVKQSHGKNLEWIGLVNPYNGGTSYNQKFKGKATLTVDKSSSTAYMELLSLKSEDSAVYYCAISRYGSESWYFDVWGAGTTVTVSS

SEQ ID NO: 62 - protein, 118 aa, Artificial Sequence
  Heavy chain variable region
QVHLQQSGAELMKPGASVKISCKATGYTFNIYWIDWVKQRPGHGLEWIGEILPGSGNTHYNENFKGKATMTADTSSNTAYMQLTSLTSEDSAVYYCARTDGRGYFDYWGQGTTLTVSS

SEQ ID NO: 63 - protein, 117 aa, Artificial Sequence
  Heavy chain variable region
QVQLQQSGAELMKPGASVKISCKATGYTFSSYWIEWVKQRPGHGLEWLGEFLPRSGKTNYNEEFRGKATFTADTSSNTAYMQLSSLTSEDSAVYYCARTDPPYFGVWGAGTMVAVSS

SEQ ID NO: 64 - protein, 106 aa, Artificial Sequence
  Light chain variable region
QIVLSQSPAILSASPGEKVTMTCRASSSISYMHWYQQKPGSSPKPWISATSNLASGVPARFSGSGSGTSYSLTISGVEAEDAATYYCQQWSSNPPTFGGGTNLEIK

SEQ ID NO: 65 - protein, 107 aa, Artificial Sequence
  Light chain variable region
SIVMTQSPKSMSMSVGERVTLSCKASENVGGYVSWYQQKPDQSPKLLIYGASSRHTGVPDRFTGSGSETDFTLTISSVQAEDLAAYHCGQNYIYPFTFGGGTKLEIK

SEQ ID NO: 66 - protein, 109 aa, Artificial Sequence
  Light chain variable region
QAVVTQESALTTSPGETVTLTCRSSTGAVTISNYANWVQEKPDHLFTGLIGGTNNRPPGVPARFSGSLIGDKAALTITGAQTEDEAIYFCVLWYSNHWVFGGGTKLTVL

SEQ ID NO: 67 - protein, 112 aa, Artificial Sequence
  Light chain variable region
DIVMTQAAFSTPVTLGTSASISCRSSQSLLHSNGITYLYWYLQKPGQSPQLLIYQMSNLASGVPDRFSSSGSGTDFTLRISRVEAEDVGVYYCAQNLEFPFTFGSGTKLEIK

SEQ ID NO: 68 - protein, 106 aa, Artificial Sequence
  Light chain variable region
QIVLTQSPALMSASPGEKVTMTCSATSSVSYIYWYQQKPRSSPKPWIYLTSNLASGVPARFSGSGSGTSYSLTISSMEAEDAATYYCQQWSSNPPTFGGGTKLEIK

SEQ ID NO: 69 - protein, 106 aa, Artificial Sequence
  Light chain variable region
DIVMTQSPAIMSASPGEKVTMTCSASSSVSYMHWYQQKSGTSPKRWIYDTSKLASGVPARFSGSGSGTSYSLTISSMETEDAATYYCQQWSSNPPTFGGGTKLELK

SEQ ID NO: 70 - protein, 106 aa, Artificial Sequence
  Light chain variable region
QIVLSQSPAILSASPGEKVTMTCRATSSVSYMYWYQQKPGSSPKPWIYATSNLASGVPARFSGSGSGTSYSLTISRVEAEDAATYYCQQWSSNPPTFGGGTKLEKK

SEQ ID NO: 71 - protein, 111 aa, Artificial Sequence
  Light chain variable region
DIVLTQSPASLVVSLGQRATISCRTSKSVSSSAYSYMHWYQQKPGQPPKVLIYLASNLESGVPARFSGSGSGTDFTLNIHPVEEEDAATYYCQHSRELPFTFGSGTKLEIK

SEQ ID NO: 72 - protein, 106 aa, Artificial Sequence
  Light chain variable region
QIVLTQSPPIMSASPGEKVTMTCSASSNISYMHWYQQKSGTSPKRWIYDTSKLASGVPARFSGSGSGTSYSLTISSMEAEDAATYYCQQWSSVPLTFGAGTKLEIK

SEQ ID NO: 73 - protein, 229 aa, Artificial Sequence
  Human IgG4 wild type Fc sequence
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 326 aa, Artificial Sequence
  Full-length sequence of the constant region of the antibody
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 75 - protein, 107 aa, Artificial Sequence [sequence omitted]

SEQ ID NO: 76 - protein, 447 aa, Artificial Sequence
  Heavy chain
EVLLQQSGPELVKPGASMKISCKASVYSFTAYTMNWVKQSHGKNLEWIGLINPHNGGTRYNQKFKGKATLTLDKSSSTAYMDLLSLTSEDSAVYYCAISRYGSSSFYFDVWGAGTTVAVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 77 - protein, 442 aa, Artificial Sequence
  Heavy chain
QVQLQQSGAELVRPGTSVKISCKASGYAFTNYWLGWMKQRPGHGLEWIGDFYPRTGNTFYNENFKGKVTLTADKSSNTAYMQLSSLTSEDSAVYLCARAGTGFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 78 - protein, 443 aa, Artificial Sequence
  Heavy chain
QVQLQQSGAELMKPGASVKISCKTTGYRFSSYWIEWVKQRPGHGLEWLGEILPGRGIINYNENFRGKATFTADTSSNTAYVQLSSLTSEDSAVYFCARTDPPYFGVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 79 - protein, 444 aa, Artificial Sequence
  Heavy chain
QVQLQQSGAELMKPGASMKISCKATGYTFSTYWIEWVKQRPGHGLEWIGENLPGRHITNYNEKFKGKATFTADTSSNTAYMQLSSLTSEDSAVYYCARGRGTYYFDYWGQGTPLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 80 - protein, 447 aa, Artificial Sequence
  Heavy chain
EVQLQQSGPELVKPGASMKISCKASGYSFTGYTMNWVKQSHGKNLEWIGLINPYNGGTNYNQKFKGKATLTVDKSSSTAYMELLSLTSEDSAVYYCAFSYYGSRGFYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 81 - protein, 448 aa, Artificial Sequence
  Heavy chain
EVQLQQSGPELVKPGASMKISCKASGYSFTGYTMNWVKQSHGKNLEWIGLINPYNGGTRYNQKFKGKATLTVDKSSSTAYMELLSLTSEDSAVYYCASSSYRNDGNWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 82 - protein, 447 aa, Artificial Sequence
  Heavy chain
EVQLQQSGPELVKPGASMKISCKASGYSITGYTMNWVKQSHGKNLEWIGLVNPYNGGTSYNQKFKGKATLTVDKSSSTAYMELLSLKSEDSAVYYCAISRYGSESWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 83 - protein, 444 aa, Artificial Sequence
  Heavy chain
QVHLQQSGAELMKPGASVKISCKATGYTFNIYWIDWVKQRPGHGLEWIGEILPGSGNTHYNENFKGKATMTADTSSNTAYMQLTSLTSEDSAVYYCARTDGRGYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 84 - protein, 443 aa, Artificial Sequence
  Heavy chain
QVQLQQSGAELMKPGASVKISCKATGYTFSSYWIEWVKQRPGHGLEWLGEFLPRSGKTNYNEEFRGKATFTADTSSNTAYMQLSSLTSEDSAVYYCARTDPPYFGVWGAGTMVAVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 85 - protein, 213 aa, Artificial Sequence
  Light chain
QIVLSQSPAILSASPGEKVTMTCRASSSISYMHWYQQKPGSSPKPWISATSNLASGVPARFSGSGSGTSYSLTISGVEAEDAATYYCQQWSSNPPTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 214 aa, Artificial Sequence
  Light chain
SIVMTQSPKSMSMSVGERVTLSCKASENVGGYVSWYQQKPDQSPKLLIYGASSRHTGVPDRFTGSGSETDFTLTISSVQAEDLAAYHCGQNYIYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 216 aa, Artificial Sequence
  Light chain
QAVVTQESALTTSPGETVTLTCRSSTGAVTISNYANWVQEKPDHLFTGLIGGTNNRPPGVPARFSGSLIGDKAALTITGAQTEDEAIYFCVLWYSNHWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 219 aa, Artificial Sequence
  Light chain
DIVMTQAAFSTPVTLGTSASISCRSSQSLLHSNGITYLYWYLQKPGQSPQLLIYQMSNLASGVPDRFSSSGSGTDFTLRISRVEAEDVGVYYCAQNLEFPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 213 aa, Artificial Sequence
  Light chain
QIVLTQSPALMSASPGEKVTMTCSATSSVSYIYWYQQKPRSSPKPWIYLTSNLASGVPARFSGSGSGTSYSLTISSMEAEDAATYYCQQWSSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 213 aa, Artificial Sequence
  Light chain
DIVMTQSPAIMSASPGEKVTMTCSASSSVSYMHWYQQKSGTSPKRWIYDTSKLASGVPARFSGSGSGTSYSLTISSMETEDAATYYCQQWSSNPPTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 213 aa, Artificial Sequence
  Light chain
QIVLSQSPAILSASPGEKVTMTCRATSSVSYMYWYQQKPGSSPKPWIYATSNLASGVPARFSGSGSGTSYSLTISRVEAEDAATYYCQQWSSNPPTFGGGTKLEKKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 218 aa, Artificial Sequence
  Light chain
DIVLTQSPASLVVSLGQRATISCRTSKSVSSSAYSYMHWYQQKPGQPPKVLIYLASNLESGVPARFSGSGSGTDFTLNIHPVEEEDAATYYCQHSRELPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 213 aa, Artificial Sequence
  Light chain
QIVLTQSPPIMSASPGEKVTMTCSASSNISYMHWYQQKSGTSPKRWIYDTSKLASGVPARFSGSGSGTSYSLTISSMEAEDAATYYCQQWSSVPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 242 aa, Artificial Sequence
  Single chain antibody fragment
QIVLSQSPAILSASPGEKVTMTCRASSSISYMHWYQQKPGSSPKPWISATSNLASGVPARFSGSGSGTSYSLTISGVEAEDAATYYCQQWSSNPPTFGGGTNLEIKGGGGSGGGGSGGGGSEVLLQQSGPELVKPGASMKISCKASVYSFTAYTMNWVKQSHGKNLEWIGLINPHNGGTRYNQKFKGKATLTLDKSSSTAYMDLLSLTSEDSAVYYCAISRYGSSSFYFDVWGAGTTVAVSS

SEQ ID NO: 95 - protein, 242 aa, Artificial Sequence
  Single chain antibody fragment
EVLLQQSGPELVKPGASMKISCKASVYSFTAYTMNWVKQSHGKNLEWIGLINPHNGGTRYNQKFKGKATLTLDKSSSTAYMDLLSLTSEDSAVYYCAISRYGSSSFYFDVWGAGTTVAVSSGGGGSGGGGSGGGGSQIVLSQSPAILSASPGEKVTMTCRASSSISYMHWYQQKPGSSPKPWISATSNLASGVPARFSGSGSGTSYSLTISGVEAEDAATYYCQQWSSNPPTFGGGTNLEIK

SEQ ID NO: 96 - protein, 238 aa, Artificial Sequence
  Single chain antibody fragment
SIVMTQSPKSMSMSVGERVTLSCKASENVGGYVSWYQQKPDQSPKLLIYGASSRHTGVPDRFTGSGSETDFTLTISSVQAEDLAAYHCGQNYIYPFTFGGGTKLEIKGGGGSGGGGSGGGGSQVQLQQSGAELVRPGTSVKISCKASGYAFTNYWLGWMKQRPGHGLEWIGDFYPRTGNTFYNENFKGKVTLTADKSSNTAYMQLSSLTSEDSAVYLCARAGTGFDYWGQGTTLTVSS

SEQ ID NO: 97 - protein, 238 aa, Artificial Sequence
  Single chain antibody fragment
QVQLQQSGAELVRPGTSVKISCKASGYAFTNYWLGWMKQRPGHGLEWIGDFYPRTGNTFYNENFKGKVTLTADKSSNTAYMQLSSLTSEDSAVYLCARAGTGFDYWGQGTTLTVSSGGGGSGGGGSGGGGSSIVMTQSPKSMSMSVGERVTLSCKASENVGGYVSWYQQKPDQSPKLLIYGASSRHTGVPDRFTGSGSETDFTLTISSVQAEDLAAYHCGQNYIYPFTFGGGTKLEIK

SEQ ID NO: 98 - protein, 241 aa, Artificial Sequence
  Single chain antibody fragment
QAVVTQESALTTSPGETVTLTCRSSTGAVTISNYANWVQEKPDHLFTGLIGGTNNRPPGVPARFSGSLIGDKAALTITGAQTEDEAIYFCVLWYSNHWVFGGGTKLTVLGGGGSGGGGSGGGGSQVQLQQSGAELMKPGASVKISCKTTGYRFSSYWIEWVKQRPGHGLEWLGEILPGRGIINYNENFRGKATFTADTSSNTAYVQLSSLTSEDSAVYFCARTDPPYFGVWGAGTTVTVSS

SEQ ID NO: 99 - protein, 241 aa, Artificial Sequence
  Single chain antibody fragment
QVQLQQSGAELMKPGASVKISCKTTGYRFSSYWIEWVKQRPGHGLEWLGEILPGRGIINYNENFRGKATFTADTSSNTAYVQLSSLTSEDSAVYFCARTDPPYFGVWGAGTTVTVSSGGGGSGGGGSGGGGSQAVVTQESALTTSPGETVTLTCRSSTGAVTISNYANWVQEKPDHLFTGLIGGTNNRPPGVPARFSGSLIGDKAALTITGAQTEDEAIYFCVLWYSNHWVFGGGTKLTVL

SEQ ID NO: 100 - protein, 245 aa, Artificial Sequence
  Single chain antibody fragment
DIVMTQAAFSTPVTLGTSASISCRSSQSLLHSNGITYLYWYLQKPGQSPQLLIYQMSNLASGVPDRFSSSGSGTDFTLRISRVEAEDVGVYYCAQNLEFPFTFGSGTKLEIKGGGGSGGGGSGGGGSQVQLQQSGAELMKPGASMKISCKATGYTFSTYWIEWVKQRPGHGLEWIGENLPGRHITNYNEKFKGKATFTADTSSNTAYMQLSSLTSEDSAVYYCARGRGTYYFDYWGQGTPLTVSS

SEQ ID NO: 101 - protein, 245 aa, Artificial Sequence
  Single chain antibody fragment
QVQLQQSGAELMKPGASMKISCKATGYTFSTYWIEWVKQRPGHGLEWIGENLPGRHITNYNEKFKGKATFTADTSSNTAYMQLSSLTSEDSAVYYCARGRGTYYFDYWGQGTPLTVSSGGGGSGGGGSGGGGSDIVMTQAAFSTPVTLGTSASISCRSSQSLLHSNGITYLYWYLQKPGQSPQLLIYQMSNLASGVPDRFSSSGSGTDFTLRISRVEAEDVGVYYCAQNLEFPFTFGSGTKLEIK

SEQ ID NO: 102 - protein, 242 aa, Artificial Sequence
  Single chain antibody fragment
QIVLTQSPALMSASPGEKVTMTCSATSSVSYIYWYQQKPRSSPKPWIYLTSNLASGVPARFSGSGSGTSYSLTISSMEAEDAATYYCQQWSSNPPTFGGGTKLEIKGGGGSGGGGSGGGGSEVQLQQSGPELVKPGASMKISCKASGYSFTGYTMNWVKQSHGKNLEWIGLINPYNGGTNYNQKFKGKATLTVDKSSSTAYMELLSLTSEDSAVYYCAFSYYGSRGFYFDYWGQGTTLTVSS

SEQ ID NO: 103 - protein, 242 aa, Artificial Sequence
  Single chain antibody fragment
EVQLQQSGPELVKPGASMKISCKASGYSFTGYTMNWVKQSHGKNLEWIGLINPYNGGTNYNQKFKGKATLTVDKSSSTAYMELLSLTSEDSAVYYCAFSYYGSRGFYFDYWGQGTTLTVSSGGGGSGGGGSGGGGSQIVLTQSPALMSASPGEKVTMTCSATSSVSYIYWYQQKPRSSPKPWIYLTSNLASGVPARFSGSGSGTSYSLTISSMEAEDAATYYCQQWSSNPPTFGGGTKLEIK

SEQ ID NO: 104 - protein, 243 aa, Artificial Sequence
  Single chain antibody fragment
DIVMTQSPAIMSASPGEKVTMTCSASSSVSYMHWYQQKSGTSPKRWIYDTSKLASGVPARFSGSGSGTSYSLTISSMETEDAATYYCQQWSSNPPTFGGGTKLELKGGGGSGGGGSGGGGSEVQLQQSGPELVKPGASMKISCKASGYSFTGYTMNWVKQSHGKNLEWIGLINPYNGGTRYNQKFKGKATLTVDKSSSTAYMELLSLTSEDSAVYYCASSSYRNDGNWYFDVWGAGTTVTVSS

SEQ ID NO: 105 - protein, 243 aa, Artificial Sequence
  Single chain antibody fragment
EVQLQQSGPELVKPGASMKISCKASGYSFTGYTMNWVKQSHGKNLEWIGLINPYNGGTRYNQKFKGKATLTVDKSSSTAYMELLSLTSEDSAVYYCASSSYRNDGNWYFDVWGAGTTVTVSSGGGGSGGGGSGGGGSDIVMTQSPAIMSASPGEKVTMTCSASSSVSYMHWYQQKSGTSPKRWIYDTSKLASGVPARFSGSGSGTSYSLTISSMETEDAATYYCQQWSSNPPTFGGGTKLELK

SEQ ID NO: 106 - protein, 242 aa, Artificial Sequence
  Single chain antibody fragment
QIVLSQSPAILSASPGEKVTMTCRATSSVSYMYWYQQKPGSSPKPWIYATSNLASGVPARFSGSGSGTSYSLTISRVEAEDAATYYCQQWSSNPPTFGGGTKLEKKGGGGSGGGGSGGGGSEVQLQQSGPELVKPGASMKISCKASGYSITGYTMNWVKQSHGKNLEWIGLVNPYNGGTSYNQKFKGKATLTVDKSSSTAYMELLSLKSEDSAVYYCAISRYGSESWYFDVWGAGTTVTVSS

SEQ ID NO: 107 - protein, 242 aa, Artificial Sequence
  Single chain antibody fragment
EVQLQQSGPELVKPGASMKISCKASGYSITGYTMNWVKQSHGKNLEWIGLVNPYNGGTSYNQKFKGKATLTVDKSSSTAYMELLSLKSEDSAVYYCAISRYGSESWYFDVWGAGTTVTVSSGGGGSGGGGSGGGGSQIVLSQSPAILSASPGEKVTMTCRATSSVSYMYWYQQKPGSSPKPWIYATSNLASGVPARFSGSGSGTSYSLTISRVEAEDAATYYCQQWSSNPPTFGGGTKLEKK

SEQ ID NO: 108 - protein, 244 aa, Artificial Sequence
  Single chain antibody fragment
DIVLTQSPASLVVSLGQRATISCRTSKSVSSSAYSYMHWYQQKPGQPPKVLIYLASNLESGVPARFSGSGSGTDFTLNIHPVEEEDAATYYCQHSRELPFTFGSGTKLEIKGGGGSGGGGSGGGGSQVHLQQSGAELMKPGASVKISCKATGYTFNIYWIDWVKQRPGHGLEWIGEILPGSGNTHYNENFKGKATMTADTSSNTAYMQLTSLTSEDSAVYYCARTDGRGYFDYWGQGTTLTVSS

SEQ ID NO: 109 - protein, 244 aa, Artificial Sequence
  Single chain antibody fragment
QVHLQQSGAELMKPGASVKISCKATGYTFNIYWIDWVKQRPGHGLEWIGEILPGSGNTHYNENFKGKATMTADTSSNTAYMQLTSLTSEDSAVYYCARTDGRGYFDYWGQGTTLTVSSGGGGSGGGGSGGGGSDIVLTQSPASLVVSLGQRATISCRTSKSVSSSAYSYMHWYQQKPGQPPKVLIYLASNLESGVPARFSGSGSGTDFTLNIHPVEEEDAATYYCQHSRELPFTFGSGTKLEIK

SEQ ID NO: 110 - protein, 238 aa, Artificial Sequence
  Single chain antibody fragment
QIVLTQSPPIMSASPGEKVTMTCSASSNISYMHWYQQKSGTSPKRWIYDTSKLASGVPARFSGSGSGTSYSLTISSMEAEDAATYYCQQWSSVPLTFGAGTKLEIKGGGGSGGGGSGGGGSQVQLQQSGAELMKPGASVKISCKATGYTFSSYWIEWVKQRPGHGLEWLGEFLPRSGKTNYNEEFRGKATFTADTSSNTAYMQLSSLTSEDSAVYYCARTDPPYFGVWGAGTMVAVSS

SEQ ID NO: 111 - protein, 238 aa, Artificial Sequence
  Single chain antibody fragment
QVQLQQSGAELMKPGASVKISCKATGYTFSSYWIEWVKQRPGHGLEWLGEFLPRSGKTNYNEEFRGKATFTADTSSNTAYMQLSSLTSEDSAVYYCARTDPPYFGVWGAGTMVAVSSGGGGSGGGGSGGGGSQIVLTQSPPIMSASPGEKVTMTCSASSNISYMHWYQQKSGTSPKRWIYDTSKLASGVPARFSGSGSGTSYSLTISSMEAEDAATYYCQQWSSVPLTFGAGTKLEIK

SEQ ID NO: 112 - DNA, 1353 bp, Artificial Sequence
  Nucleic acid molecule
gaagtgctgctgcagcagagcggaccagagctggtcaagcccggcgcctccatgaagatcagctgtaaggcctccgtgtacagcttcacagcctacacaatgaactgggtgaagcagagccacggcaagaatctggagtggatcggactgatcaacccacacaatggcggcactaggtacaaccagaagttcaagggcaaggccacactgactctggataagtccagcagcactgcctacatggatctgctgtctctgacaagcgaggacagcgccgtctactattgcgccatctctaggtacggcagcagcagcttctacttcgatgtgtggggcgccggcacaacagtggctgtgagcagcgccagcaccaagggccccagcgtgttccctctggctccttgtagccggtccacctccgagtccacagctgctctgggctgcctcgtgaaggactactttcccgaacccgttaccgtgagctggaatagcggcgctttaacctccggagtgcacaccttccccgctgtgctccagtcctccggtttatactctttatcctccgtggtgaccgtgccttcctccagcctcggcaccaagacctacacttgtaacgtggaccacaagcccagcaacaccaaggtggacaagagggtggagtccaagtacggacctccttgtcccccttgccccgcccccgaggccgctggcggaccctccgtgttcctcttcccccccaaacccaaggacactttaatgatctcccggacccccgaagtgacttgtgtggtggtggacgtgtcccaagaagaccccgaggtgcagtttaactggtacgtggatggcgtggaggtgcacaacgccaagaccaagcctagggaggaacagttcaactccacctaccgggtggtgtccgtgctcaccgtgctgcatcaagattggctgaacggcaaggagtacaagtgcaaggtgagcaacaagggactgcccagctccatcgagaagaccatcagcaaggccaaaggccagccccgggaacctcaagtttatacactgccccccagccaagaagagatgaccaagaaccaagtttctttaacttgtttagtgaagggcttctaccctagcgacatcgctgtggagtgggagtccaatggccagcccgaaaacaattataagaccaccccccccgtgctggactccgatggttctttttttttatactccaagctgacagtggacaagtctcgttggcaagaaggcaacgtgttctcttgtagcgtgatgcacgaggctttacacaaccactacacccagaagtctttatctctgtctttaggctgatgagaattc

SEQ ID NO: 113 - DNA, 1338 bp, Artificial Sequence
  Nucleic acid molecule
caagtgcagctccagcagagcggagctgagctggtgagacccggcactagcgtgaagatcagctgtaaggccagcggctacgccttcactaattactggctgggctggatgaagcagagacccggccatggactggagtggatcggcgacttctacccaaggactggcaacactttctacaacgagaactttaagggcaaggtgactctgactgccgataagtccagcaacactgcctacatgcagctgtcctctctgactagcgaagatagcgccgtgtatctgtgtgctagggctggcactggcttcgattactggggccaaggcacaacactgacagtgagcagcgccagcaccaagggccccagcgtgttccctctggctccttgtagccggtccacctccgagtccacagctgctctgggctgcctcgtgaaggactactttcccgaacccgttaccgtgagctggaatagcggcgctttaacctccggagtgcacaccttccccgctgtgctccagtcctccggtttatactctttatcctccgtggtgaccgtgccttcctccagcctcggcaccaagacctacacttgtaacgtggaccacaagcccagcaacaccaaggtggacaagagggtggagtccaagtacggacctccttgtcccccttgccccgcccccgaggccgctggcggaccctccgtgttcctcttcccccccaaacccaaggacactttaatgatctcccggacccccgaagtgacttgtgtggtggtggacgtgtcccaagaagaccccgaggtgcagtttaactggtacgtggatggcgtggaggtgcacaacgccaagaccaagcctagggaggaacagttcaactccacctaccgggtggtgtccgtgctcaccgtgctgcatcaagattggctgaacggcaaggagtacaagtgcaaggtgagcaacaagggactgcccagctccatcgagaagaccatcagcaaggccaaaggccagccccgggaacctcaagtttatacactgccccccagccaagaagagatgaccaagaaccaagtttctttaacttgtttagtgaagggcttctaccctagcgacatcgctgtggagtgggagtccaatggccagcccgaaaacaattataagaccaccccccccgtgctggactccgatggttctttttttttatactccaagctgacagtggacaagtctcgttggcaagaaggcaacgtgttctcttgtagcgtgatgcacgaggctttacacaaccactacacccagaagtctttatctctgtctttaggctgatgagaattc

SEQ ID NO: 114 - DNA, 1341 bp, Artificial Sequence
  Nucleic acid molecule
caagtgcagctgcagcagagcggcgctgagctgatgaagcccggcgcttccgtcaagatcagctgcaagactactggctataggttcagcagctactggatcgagtgggtgaagcagagacccggccacggactggaatggctgggcgaaatcctccccggaaggggcatcatcaactacaatgagaactttaggggcaaggccacattcacagccgacacaagcagcaacactgcctacgtgcagctgagctctctgacttccgaggacagcgccgtgtacttctgcgccagaacagaccctccttatttcggcgtgtggggcgccggaactactgtgactgtgtccagcgccagcaccaagggccccagcgtgttccctctggctccttgtagccggtccacctccgagtccacagctgctctgggctgcctcgtgaaggactactttcccgaacccgttaccgtgagctggaatagcggcgctttaacctccggagtgcacaccttccccgctgtgctccagtcctccggtttatactctttatcctccgtggtgaccgtgccttcctccagcctcggcaccaagacctacacttgtaacgtggaccacaagcccagcaacaccaaggtggacaagagggtggagtccaagtacggacctccttgtcccccttgccccgcccccgaggccgctggcggaccctccgtgttcctcttcccccccaaacccaaggacactttaatgatctcccggacccccgaagtgacttgtgtggtggtggacgtgtcccaagaagaccccgaggtgcagtttaactggtacgtggatggcgtggaggtgcacaacgccaagaccaagcctagggaggaacagttcaactccacctaccgggtggtgtccgtgctcaccgtgctgcatcaagattggctgaacggcaaggagtacaagtgcaaggtgagcaacaagggactgcccagctccatcgagaagaccatcagcaaggccaaaggccagccccgggaacctcaagtttatacactgccccccagccaagaagagatgaccaagaaccaagtttctttaacttgtttagtgaagggcttctaccctagcgacatcgctgtggagtgggagtccaatggccagcccgaaaacaattataagaccaccccccccgtgctggactccgatggttctttttttttatactccaagctgacagtggacaagtctcgttggcaagaaggcaacgtgttctcttgtagcgtgatgcacgaggctttacacaaccactacacccagaagtctttatctctgtctttaggctgatgagaattc

SEQ ID NO: 115 - DNA, 1344 bp, Artificial Sequence
  Nucleic acid molecule
caagtgcagctgcagcagagcggagccgagctgatgaagcccggcgcctccatgaagatcagctgcaaggccactggctacacattcagcacatactggatcgagtgggtcaagcagagacccggccacggactggagtggatcggagagaatctgcccggaaggcacatcactaactacaacgagaagttcaagggcaaggccactttcacagccgacactagcagcaacactgcctacatgcagctcagctctctgacaagcgaagatagcgccgtgtactactgtgctaggggaaggggcacttactacttcgattactggggccaaggcactccactgactgtgtccagcgccagcaccaagggccccagcgtgttccctctggctccttgtagccggtccacctccgagtccacagctgctctgggctgcctcgtgaaggactactttcccgaacccgttaccgtgagctggaatagcggcgctttaacctccggagtgcacaccttccccgctgtgctccagtcctccggtttatactctttatcctccgtggtgaccgtgccttcctccagcctcggcaccaagacctacacttgtaacgtggaccacaagcccagcaacaccaaggtggacaagagggtggagtccaagtacggacctccttgtcccccttgccccgcccccgaggccgctggcggaccctccgtgttcctcttcccccccaaacccaaggacactttaatgatctcccggacccccgaagtgacttgtgtggtggtggacgtgtcccaagaagaccccgaggtgcagtttaactggtacgtggatggcgtggaggtgcacaacgccaagaccaagcctagggaggaacagttcaactccacctaccgggtggtgtccgtgctcaccgtgctgcatcaagattggctgaacggcaaggagtacaagtgcaaggtgagcaacaagggactgcccagctccatcgagaagaccatcagcaaggccaaaggccagccccgggaacctcaagtttatacactgccccccagccaagaagagatgaccaagaaccaagtttctttaacttgtttagtgaagggcttctaccctagcgacatcgctgtggagtgggagtccaatggccagcccgaaaacaattataagaccaccccccccgtgctggactccgatggttctttttttttatactccaagctgacagtggacaagtctcgttggcaagaaggcaacgtgttctcttgtagcgtgatgcacgaggctttacacaaccactacacccagaagtctttatctctgtctttaggctgatgagaattc

SEQ ID NO: 116 - DNA, 1353 bp, Artificial Sequence
  Nucleic acid molecule
gaggtgcagctccaacagagcggaccagagctggtgaaacccggcgccagcatgaagatcagctgtaaggcctccggctacagcttcactggctacactatgaactgggtgaagcagtcccacggcaagaatctggagtggatcggactgatcaacccatacaacggcggcacaaactacaaccagaagttcaagggcaaggccactctgacagtcgataagagctccagcactgcctacatggagctgctgagcctcactagcgaggacagcgctgtgtactactgtgccttctcctactacggctctaggggcttctacttcgattactggggccaaggcacaacactgacagtgtccagcgccagcaccaagggccccagcgtgttccctctggctccttgtagccggtccacctccgagtccacagctgctctgggctgcctcgtgaaggactactttcccgaacccgttaccgtgagctggaatagcggcgctttaacctccggagtgcacaccttccccgctgtgctccagtcctccggtttatactctttatcctccgtggtgaccgtgccttcctccagcctcggcaccaagacctacacttgtaacgtggaccacaagcccagcaacaccaaggtggacaagagggtggagtccaagtacggacctccttgtcccccttgccccgcccccgaggccgctggcggaccctccgtgttcctcttcccccccaaacccaaggacactttaatgatctcccggacccccgaagtgacttgtgtggtggtggacgtgtcccaagaagaccccgaggtgcagtttaactggtacgtggatggcgtggaggtgcacaacgccaagaccaagcctagggaggaacagttcaactccacctaccgggtggtgtccgtgctcaccgtgctgcatcaagattggctgaacggcaaggagtacaagtgcaaggtgagcaacaagggactgcccagctccatcgagaagaccatcagcaaggccaaaggccagccccgggaacctcaagtttatacactgccccccagccaagaagagatgaccaagaaccaagtttctttaacttgtttagtgaagggcttctaccctagcgacatcgctgtggagtgggagtccaatggccagcccgaaaacaattataagaccaccccccccgtgctggactccgatggttctttttttttatactccaagctgacagtggacaagtctcgttggcaagaaggcaacgtgttctcttgtagcgtgatgcacgaggctttacacaaccactacacccagaagtctttatctctgtctttaggctgatgagaattc

SEQ ID NO: 117 - DNA, 1356 bp, Artificial Sequence
  Nucleic acid molecule
gaagtgcagctgcaacagagcggcccagagctcgtgaagcccggcgccagcatgaagatcagctgcaaggccagcggctacagcttcactggctacacaatgaactgggtcaagcagagccacggaaaaaatctggagtggatcggactgatcaacccttacaacggcggcacaaggtacaatcagaagttcaagggcaaggccactctcactgtggataagagcagcagcactgcctacatggagctgctgtctctgacaagcgaggatagcgccgtgtactactgcgccagctcctcctataggaacgacggcaactggtacttcgatgtgtggggcgccggcactactgtgacagtgagctccgccagcaccaagggccccagcgtgttccctctggctccttgtagccggtccacctccgagtccacagctgctctgggctgcctcgtgaaggactactttcccgaacccgttaccgtgagctggaatagcggcgctttaacctccggagtgcacaccttccccgctgtgctccagtcctccggtttatactctttatcctccgtggtgaccgtgccttcctccagcctcggcaccaagacctacacttgtaacgtggaccacaagcccagcaacaccaaggtggacaagagggtggagtccaagtacggacctccttgtcccccttgccccgcccccgaggccgctggcggaccctccgtgttcctcttcccccccaaacccaaggacactttaatgatctcccggacccccgaagtgacttgtgtggtggtggacgtgtcccaagaagaccccgaggtgcagtttaactggtacgtggatggcgtggaggtgcacaacgccaagaccaagcctagggaggaacagttcaactccacctaccgggtggtgtccgtgctcaccgtgctgcatcaagattggctgaacggcaaggagtacaagtgcaaggtgagcaacaagggactgcccagctccatcgagaagaccatcagcaaggccaaaggccagccccgggaacctcaagtttatacactgccccccagccaagaagagatgaccaagaaccaagtttctttaacttgtttagtgaagggcttctaccctagcgacatcgctgtggagtgggagtccaatggccagcccgaaaacaattataagaccaccccccccgtgctggactccgatggttctttttttttatactccaagctgacagtggacaagtctcgttggcaagaaggcaacgtgttctcttgtagcgtgatgcacgaggctttacacaaccactacacccagaagtctttatctctgtctttaggctgatgagaattc

SEQ ID NO: 118 - DNA, 1353 bp, Artificial Sequence
  Nucleic acid molecule
gaagtgcagctgcagcagagcggaccagagctggtcaagcccggcgccagcatgaagatcagctgtaaggccagcggctacagcatcactggctacactatgaactgggtgaagcagagccacggcaagaacctcgagtggattggcctcgtgaacccatacaacggcggcacttcctacaaccagaagttcaaaggcaaggccacactcacagtcgataagtccagctccacagcctacatggagctgctgtctctgaagagcgaggatagcgctgtctactactgtgccatcagcagatacggcagcgagagctggtacttcgacgtgtggggcgccggcacaacagtgacagtgagcagcgccagcacaaagggccccagcgtgttccctctggctccttgtagccggtccacctccgagtccacagctgctctgggctgcctcgtgaaggactactttcccgaacccgttaccgtgagctggaatagcggcgctttaacctccggagtgcacaccttccccgctgtgctccagtcctccggtttatactctttatcctccgtggtgaccgtgccttcctccagcctcggcaccaagacctacacttgtaacgtggaccacaagcccagcaacaccaaggtggacaagagggtggagtccaagtacggacctccttgtcccccttgccccgcccccgaggccgctggcggaccctccgtgttcctcttcccccccaaacccaaggacactttaatgatctcccggacccccgaagtgacttgtgtggtggtggacgtgtcccaagaagaccccgaggtgcagtttaactggtacgtggatggcgtggaggtgcacaacgccaagaccaagcctagggaggaacagttcaactccacctaccgggtggtgtccgtgctcaccgtgctgcatcaagattggctgaacggcaaggagtacaagtgcaaggtgagcaacaagggactgcccagctccatcgagaagaccatcagcaaggccaaaggccagccccgggaacctcaagtttatacactgccccccagccaagaagagatgaccaagaaccaagtttctttaacttgtttagtgaagggcttctaccctagcgacatcgctgtggagtgggagtccaatggccagcccgaaaacaattataagaccaccccccccgtgctggactccgatggttctttttttttatactccaagctgacagtggacaagtctcgttggcaagaaggcaacgtgttctcttgtagcgtgatgcacgaggctttacacaaccactacacccagaagtctttatctctgtctttaggctgatgagaattc

SEQ ID NO: 119 - DNA, 1344 bp, Artificial Sequence
  Nucleic acid molecule
caagtgcatctgcagcagagcggcgctgagctgatgaagcccggcgccagcgtgaagattagctgcaaggccactggctacacattcaacatctactggatcgactgggtgaagcagaggcccggccacggactggaatggatcggcgaaattctgcccggcagcggcaacactcactacaacgagaacttcaagggcaaggccacaatgacagccgacacaagctccaacactgcttacatgcagctgacttctctgactagcgaggacagcgccgtgtactattgcgctaggacagacggaaggggctacttcgattactggggccaaggcactacactcacagtgagcagcgccagcactaagggccccagcgtgttccctctggctccttgtagccggtccacctccgagtccacagctgctctgggctgcctcgtgaaggactactttcccgaacccgttaccgtgagctggaatagcggcgctttaacctccggagtgcacaccttccccgctgtgctccagtcctccggtttatactctttatcctccgtggtgaccgtgccttcctccagcctcggcaccaagacctacacttgtaacgtggaccacaagcccagcaacaccaaggtggacaagagggtggagtccaagtacggacctccttgtcccccttgccccgcccccgaggccgctggcggaccctccgtgttcctcttcccccccaaacccaaggacactttaatgatctcccggacccccgaagtgacttgtgtggtggtggacgtgtcccaagaagaccccgaggtgcagtttaactggtacgtggatggcgtggaggtgcacaacgccaagaccaagcctagggaggaacagttcaactccacctaccgggtggtgtccgtgctcaccgtgctgcatcaagattggctgaacggcaaggagtacaagtgcaaggtgagcaacaagggactgcccagctccatcgagaagaccatcagcaaggccaaaggccagccccgggaacctcaagtttatacactgccccccagccaagaagagatgaccaagaaccaagtttctttaacttgtttagtgaagggcttctaccctagcgacatcgctgtggagtgggagtccaatggccagcccgaaaacaattataagaccaccccccccgtgctggactccgatggttctttttttttatactccaagctgacagtggacaagtctcgttggcaagaaggcaacgtgttctcttgtagcgtgatgcacgaggctttacacaaccactacacccagaagtctttatctctgtctttaggctgatgagaattc

SEQ ID NO: 120 - DNA, 1341 bp, Artificial Sequence
  Nucleic acid molecule
caggtgcagctgcagcagtccggcgctgagctcatgaagcccggcgccagcgtgaagatcagctgcaaggccactggctacacattcagcagctactggatcgagtgggtgaagcagagacccggccacggactggaatggctgggagagtttctgcctagaagcggcaagacaaactacaacgaggagtttaggggcaaggctacattcactgccgacacatccagcaacactgcctacatgcagctgagcagcctcacaagcgaggattccgccgtctactactgtgctaggactgatcctccttacttcggagtgtggggcgctggcacaatggtggctgtgagcagcgcctccactaagggccccagcgtgttccctctggctccttgtagccggtccacctccgagtccacagctgctctgggctgcctcgtgaaggactactttcccgaacccgttaccgtgagctggaatagcggcgctttaacctccggagtgcacaccttccccgctgtgctccagtcctccggtttatactctttatcctccgtggtgaccgtgccttcctccagcctcggcaccaagacctacacttgtaacgtggaccacaagcccagcaacaccaaggtggacaagagggtggagtccaagtacggacctccttgtcccccttgccccgcccccgaggccgctggcggaccctccgtgttcctcttcccccccaaacccaaggacactttaatgatctcccggacccccgaagtgacttgtgtggtggtggacgtgtcccaagaagaccccgaggtgcagtttaactggtacgtggatggcgtggaggtgcacaacgccaagaccaagcctagggaggaacagttcaactccacctaccgggtggtgtccgtgctcaccgtgctgcatcaagattggctgaacggcaaggagtacaagtgcaaggtgagcaacaagggactgcccagctccatcgagaagaccatcagcaaggccaaaggccagccccgggaacctcaagtttatacactgccccccagccaagaagagatgaccaagaaccaagtttctttaacttgtttagtgaagggcttctaccctagcgacatcgctgtggagtgggagtccaatggccagcccgaaaacaattataagaccaccccccccgtgctggactccgatggttctttttttttatactccaagctgacagtggacaagtctcgttggcaagaaggcaacgtgttctcttgtagcgtgatgcacgaggctttacacaaccactacacccagaagtctttatctctgtctttaggctgatgagaattc

SEQ ID NO: 121 - DNA, 651 bp, Artificial Sequence
  Nucleic acid molecule
cagatcgtgctgagccagagcccagctattctgtccgccagccccggcgagaaggtgactatgacttgtagggccagctccagcatcagctacatgcactggtaccagcagaagcccggctcctccccaaagccttggatcagcgccactagcaatctggccagcggcgtgccagccagattcagcggaagcggcagcggcactagctactctctgactatctccggcgtggaagctgaggatgccgccacttactactgccagcagtggtccagcaaccctcctactttcggcggcggcacaaatctggagatcaagcggaccgtggctgccccctccgtgttcatcttccccccttccgacgagcagctgaagtccggcaccgctagcgtggtgtgtttactgaacaacttctaccctcgtgaggccaaggtgcagtggaaggtggacaacgctttacagtccggcaactcccaagaatccgtgaccgagcaagattccaaggactccacctactctttatcctccactttaactttatccaaggccgactacgagaagcacaaggtgtacgcttgtgaggtgacccatcaaggtttatcctcccccgtgaccaagtccttcaatcgtggcgagtgctgatgagaattc

SEQ ID NO: 122 - DNA, 654 bp, Artificial Sequence
  Nucleic acid molecule
agcatcgtcatgacacaaagccctaagagcatgagcatgagcgtgggcgagagagtgactctgagctgtaaggccagcgagaatgtgggcggctacgtgagctggtatcagcagaagccagatcagagcccaaagctgctgatctacggcgccagcagcagacacactggcgtgccagataggttcactggcagcggctccgagacagacttcactctgactatcagcagcgtccaagccgaagatctggccgcctatcactgcggccagaactacatctacccattcacattcggcggcggcacaaagctggagatcaagcggaccgtggctgccccctccgtgttcatcttccccccttccgacgagcagctgaagtccggcaccgctagcgtggtgtgtttactgaacaacttctaccctcgtgaggccaaggtgcagtggaaggtggacaacgctttacagtccggcaactcccaagaatccgtgaccgagcaagattccaaggactccacctactctttatcctccactttaactttatccaaggccgactacgagaagcacaaggtgtacgcttgtgaggtgacccatcaaggtttatcctcccccgtgaccaagtccttcaatcgtggcgagtgctgatgagaattc

SEQ ID NO: 123 - DNA, 660 bp, Artificial Sequence
  Nucleic acid molecule
caagccgtcgtgacacaagagtccgctctgacaacttcccccggcgagactgtgacactgacttgtaggagcagcactggcgccgtgactatcagcaactacgccaactgggtccaagagaagccagatcatctgttcactggactgatcggcggcacaaataataggcctcccggcgtgccagccagatttagcggctctctgattggcgataaggctgctctgacaatcactggcgcccagactgaggacgaggccatctacttctgcgtcctctggtacagcaaccactgggtgttcggcggcggcactaagctgactgtgctgcggaccgtggctgccccctccgtgttcatcttccccccttccgacgagcagctgaagtccggcaccgctagcgtggtgtgtttactgaacaacttctaccctcgtgaggccaaggtgcagtggaaggtggacaacgctttacagtccggcaactcccaagaatccgtgaccgagcaagattccaaggactccacctactctttatcctccactttaactttatccaaggccgactacgagaagcacaaggtgtacgcttgtgaggtgacccatcaaggtttatcctcccccgtgaccaagtccttcaatcgtggcgagtgctgatgagaattc

SEQ ID NO: 124 - DNA, 669 bp, Artificial Sequence
  Nucleic acid molecule
gatatcgtcatgactcaagccgccttcagcactccagtcactctcggcacaagcgccagcatcagctgtaggtccagccagtctctgctgcacagcaacggcatcacttatctgtactggtatctgcagaagcccggccaaagcccacagctgctgatctaccagatgagcaatctggccagcggcgtgccagatagattcagcagcagcggaagcggaacagacttcacactgaggatctctagggtggaagccgaagatgtgggcgtctattactgcgcccagaacctcgagttccctttcacattcggcagcggcactaagctggagatcaagcggaccgtggctgccccctccgtgttcatcttccccccttccgacgagcagctgaagtccggcaccgctagcgtggtgtgtttactgaacaacttctaccctcgtgaggccaaggtgcagtggaaggtggacaacgctttacagtccggcaactcccaagaatccgtgaccgagcaagattccaaggactccacctactctttatcctccactttaactttatccaaggccgactacgagaagcacaaggtgtacgcttgtgaggtgacccatcaaggtttatcctcccccgtgaccaagtccttcaatcgtggcgagtgctgatgagaattc

SEQ ID NO: 125 - DNA, 651 bp, Artificial Sequence
  Nucleic acid molecule
cagatcgtgctgacacagagcccagctctgatgagcgccagccccggcgagaaggtcacaatgacttgcagcgccacatccagcgtgagctacatctactggtaccagcagaagcctaggagcagccctaagccttggatctacctcacaagcaatctggccagcggagtgccagctaggttcagcggaagcggcagcggcacaagctactctctgacaatctccagcatggaagccgaagatgccgccacttactactgccagcagtggagcagcaatccacctacattcggaggcggcactaagctggagatcaagcggaccgtggctgccccctccgtgttcatcttccccccttccgacgagcagctgaagtccggcaccgctagcgtggtgtgtttactgaacaacttctaccctcgtgaggccaaggtgcagtggaaggtggacaacgctttacagtccggcaactcccaagaatccgtgaccgagcaagattccaaggactccacctactctttatcctccactttaactttatccaaggccgactacgagaagcacaaggtgtacgcttgtgaggtgacccatcaaggtttatcctcccccgtgaccaagtccttcaatcgtggcgagtgctgatgagaattc

SEQ ID NO: 126 - DNA, 651 bp, Artificial Sequence
  Nucleic acid molecule
gatatcgtgatgactcagtccccagccatcatgtccgccagccccggcgagaaggtgactatgacttgctccgccagcagcagcgtgagctacatgcactggtaccagcagaagagcggcacatccccaaagaggtggatctacgacacaagcaagctggccagcggcgtgccagccagattcagcggctccggcagcggaacaagctactctctgactatcagcagcatggagacagaggacgctgccacttactactgccagcagtggagcagcaatcctccaactttcggcggaggcactaagctggagctgaagcggaccgtggctgccccctccgtgttcatcttccccccttccgacgagcagctgaagtccggcaccgctagcgtggtgtgtttactgaacaacttctaccctcgtgaggccaaggtgcagtggaaggtggacaacgctttacagtccggcaactcccaagaatccgtgaccgagcaagattccaaggactccacctactctttatcctccactttaactttatccaaggccgactacgagaagcacaaggtgtacgcttgtgaggtgacccatcaaggtttatcctcccccgtgaccaagtccttcaatcgtggcgagtgctgatgagaattc

SEQ ID NO: 127 - DNA, 651 bp, Artificial Sequence
  Nucleic acid molecule
cagattgtgctgtcccagtccccagccattctgagcgccagccccggcgagaaggtgactatgacttgtagggccacaagcagcgtgagctacatgtactggtaccagcagaagcccggcagcagccctaagccttggatctacgccacaagcaatctggccagcggcgtcccagctagatttagcggcagcggatccggcactagctattctctgactatctctagggtcgaggccgaagatgccgccacatactactgccagcagtggtcctccaatcctccaacattcggcggcggaactaagctggagaagaagaggacagtggctgccccttccgtgttcatcttccctccaagcgacgagcagctgaagtccggcactgctagcgtggtgtgtttactgaacaacttctaccctcgtgaggccaaggtgcagtggaaggtggacaacgctttacagtccggcaactcccaagaatccgtgaccgagcaagattccaaggactccacctactctttatcctccactttaactttatccaaggccgactacgagaagcacaaggtgtacgcttgtgaggtgacccatcaaggtttatcctcccccgtgaccaagtccttcaatcgtggcgagtgctgatgagaattc

SEQ ID NO: 128 - DNA, 666 bp, Artificial Sequence
  Nucleic acid molecule
gatatcgtgctgactcagagcccagcctctctggtggtgtctctgggacagagggccacaatcagctgtaggacttccaagagcgtgagcagctccgcctacagctacatgcactggtaccagcagaagcccggccagcctcctaaggtgctgatctatctggccagcaatctggagagcggcgtcccagctagattcagcggctccggaagcggcactgacttcactctgaacatccacccagtggaagaggaggatgccgccacatactactgccagcactctagggagctgcctttcacatttggcagcggaactaagctggagatcaagaggactgtcgccgcccctagcgtgttcatcttccctccaagcgatgagcagctgaagagcggcactgctagcgtggtgtgtttactgaacaacttctaccctcgtgaggccaaggtgcagtggaaggtggacaacgctttacagtccggcaactcccaagaatccgtgaccgagcaagattccaaggactccacctactctttatcctccactttaactttatccaaggccgactacgagaagcacaaggtgtacgcttgtgaggtgacccatcaaggtttatcctcccccgtgaccaagtccttcaatcgtggcgagtgctgatgagaattc

SEQ ID NO: 129 - DNA, 651 bp, Artificial Sequence
  Nucleic acid molecule
caaattgttctcacacagagccctcctatcatgagcgccagccccggcgagaaggtgactatgacttgttccgccagcagcaacatcagctacatgcactggtaccagcagaagtccggcacaagcccaaagaggtggatctacgacacaagcaagctggccagcggcgtgccagccagatttagcggctccggaagcggcactagctactctctgacaatcagcagcatggaagccgaggacgccgctacatactactgccagcagtggagctccgtcccactgactttcggcgctggcactaagctggagatcaagaggactgtggccgccccttccgtgttcatcttccctcctagcgacgaacagctcaagagcggcactgctagcgtggtgtgtttactgaacaacttctaccctcgtgaggccaaggtgcagtggaaggtggacaacgctttacagtccggcaactcccaagaatccgtgaccgagcaagattccaaggactccacctactctttatcctccactttaactttatccaaggccgactacgagaagcacaaggtgtacgcttgtgaggtgacccatcaaggtttatcctcccccgtgaccaagtccttcaatcgtggcgagtgctgatgagaattc

SEQ ID NO: 130 - DNA, 726 bp, Artificial Sequence
  Nucleic acid molecule
cagatcgtgctgagccagagcccagctattctgtccgccagccccggcgagaaggtgactatgacttgtagggccagctccagcatcagctacatgcactggtaccagcagaagcccggctcctccccaaagccttggatcagcgccactagcaatctggccagcggcgtgccagccagattcagcggaagcggcagcggcactagctactctctgactatctccggcgtggaagctgaggatgccgccacttactactgccagcagtggtccagcaaccctcctactttcggcggcggcacaaatctggagatcaagggtggcggtggctcgggcggtggtgggtcgggtggcggcggatctgaagtgctgctgcagcagagcggaccagagctggtcaagcccggcgcctccatgaagatcagctgtaaggcctccgtgtacagcttcacagcctacacaatgaactgggtgaagcagagccacggcaagaatctggagtggatcggactgatcaacccacacaatggcggcactaggtacaaccagaagttcaagggcaaggccacactgactctggataagtccagcagcactgcctacatggatctgctgtctctgacaagcgaggacagcgccgtctactattgcgccatctctaggtacggcagcagcagcttctacttcgatgtgtggggcgccggcacaacagtggctgtgagcagc

SEQ ID NO: 131 - DNA, 726 bp, Artificial Sequence
  Nucleic acid molecule
gaagtgctgctgcagcagagcggaccagagctggtcaagcccggcgcctccatgaagatcagctgtaaggcctccgtgtacagcttcacagcctacacaatgaactgggtgaagcagagccacggcaagaatctggagtggatcggactgatcaacccacacaatggcggcactaggtacaaccagaagttcaagggcaaggccacactgactctggataagtccagcagcactgcctacatggatctgctgtctctgacaagcgaggacagcgccgtctactattgcgccatctctaggtacggcagcagcagcttctacttcgatgtgtggggcgccggcacaacagtggctgtgagcagcggtggcggtggctcgggcggtggtgggtcgggtggcggcggatctcagatcgtgctgagccagagcccagctattctgtccgccagccccggcgagaaggtgactatgacttgtagggccagctccagcatcagctacatgcactggtaccagcagaagcccggctcctccccaaagccttggatcagcgccactagcaatctggccagcggcgtgccagccagattcagcggaagcggcagcggcactagctactctctgactatctccggcgtggaagctgaggatgccgccacttactactgccagcagtggtccagcaaccctcctactttcggcggcggcacaaatctggagatcaag

SEQ ID NO: 132 - DNA, 714 bp, Artificial Sequence
  Nucleic acid molecule
agcatcgtcatgacacaaagccctaagagcatgagcatgagcgtgggcgagagagtgactctgagctgtaaggccagcgagaatgtgggcggctacgtgagctggtatcagcagaagccagatcagagcccaaagctgctgatctacggcgccagcagcagacacactggcgtgccagataggttcactggcagcggctccgagacagacttcactctgactatcagcagcgtccaagccgaagatctggccgcctatcactgcggccagaactacatctacccattcacattcggcggcggcacaaagctggagatcaagggtggcggtggctcgggcggtggtgggtcgggtggcggcggatctcaagtgcagctccagcagagcggagctgagctggtgagacccggcactagcgtgaagatcagctgtaaggccagcggctacgccttcactaattactggctgggctggatgaagcagagacccggccatggactggagtggatcggcgacttctacccaaggactggcaacactttctacaacgagaactttaagggcaaggtgactctgactgccgataagtccagcaacactgcctacatgcagctgtcctctctgactagcgaagatagcgccgtgtatctgtgtgctagggctggcactggcttcgattactggggccaaggcacaacactgacagtgagcagc

SEQ ID NO: 133 - DNA, 714 bp, Artificial Sequence
  Nucleic acid molecule
caagtgcagctccagcagagcggagctgagctggtgagacccggcactagcgtgaagatcagctgtaaggccagcggctacgccttcactaattactggctgggctggatgaagcagagacccggccatggactggagtggatcggcgacttctacccaaggactggcaacactttctacaacgagaactttaagggcaaggtgactctgactgccgataagtccagcaacactgcctacatgcagctgtcctctctgactagcgaagatagcgccgtgtatctgtgtgctagggctggcactggcttcgattactggggccaaggcacaacactgacagtgagcagcggtggcggtggctcgggcggtggtgggtcgggtggcggcggatctagcatcgtcatgacacaaagccctaagagcatgagcatgagcgtgggcgagagagtgactctgagctgtaaggccagcgagaatgtgggcggctacgtgagctggtatcagcagaagccagatcagagcccaaagctgctgatctacggcgccagcagcagacacactggcgtgccagataggttcactggcagcggctccgagacagacttcactctgactatcagcagcgtccaagccgaagatctggccgcctatcactgcggccagaactacatctacccattcacattcggcggcggcacaaagctggagatcaag

SEQ ID NO: 134 - DNA, 723 bp, Artificial Sequence
  Nucleic acid molecule
caagccgtcgtgacacaagagtccgctctgacaacttcccccggcgagactgtgacactgacttgtaggagcagcactggcgccgtgactatcagcaactacgccaactgggtccaagagaagccagatcatctgttcactggactgatcggcggcacaaataataggcctcccggcgtgccagccagatttagcggctctctgattggcgataaggctgctctgacaatcactggcgcccagactgaggacgaggccatctacttctgcgtcctctggtacagcaaccactgggtgttcggcggcggcactaagctgactgtgctgggtggcggtggctcgggcggtggtgggtcgggtggcggcggatctcaagtgcagctgcagcagagcggcgctgagctgatgaagcccggcgcttccgtcaagatcagctgcaagactactggctataggttcagcagctactggatcgagtgggtgaagcagagacccggccacggactggaatggctgggcgaaatcctccccggaaggggcatcatcaactacaatgagaactttaggggcaaggccacattcacagccgacacaagcagcaacactgcctacgtgcagctgagctctctgacttccgaggacagcgccgtgtacttctgcgccagaacagaccctccttatttcggcgtgtggggcgccggaactactgtgactgtgtccagc

SEQ ID NO: 135 - DNA, 723 bp, Artificial Sequence
  Nucleic acid molecule
caagtgcagctgcagcagagcggcgctgagctgatgaagcccggcgcttccgtcaagatcagctgcaagactactggctataggttcagcagctactggatcgagtgggtgaagcagagacccggccacggactggaatggctgggcgaaatcctccccggaaggggcatcatcaactacaatgagaactttaggggcaaggccacattcacagccgacacaagcagcaacactgcctacgtgcagctgagctctctgacttccgaggacagcgccgtgtacttctgcgccagaacagaccctccttatttcggcgtgtggggcgccggaactactgtgactgtgtccagcggtggcggtggctcgggcggtggtgggtcgggtggcggcggatctcaagccgtcgtgacacaagagtccgctctgacaacttcccccggcgagactgtgacactgacttgtaggagcagcactggcgccgtgactatcagcaactacgccaactgggtccaagagaagccagatcatctgttcactggactgatcggcggcacaaataataggcctcccggcgtgccagccagatttagcggctctctgattggcgataaggctgctctgacaatcactggcgcccagactgaggacgaggccatctacttctgcgtcctctggtacagcaaccactgggtgttcggcggcggcactaagctgactgtgctg

SEQ ID NO: 136 - DNA, 735 bp, Artificial Sequence
  Nucleic acid molecule
gatatcgtcatgactcaagccgccttcagcactccagtcactctcggcacaagcgccagcatcagctgtaggtccagccagtctctgctgcacagcaacggcatcacttatctgtactggtatctgcagaagcccggccaaagcccacagctgctgatctaccagatgagcaatctggccagcggcgtgccagatagattcagcagcagcggaagcggaacagacttcacactgaggatctctagggtggaagccgaagatgtgggcgtctattactgcgcccagaacctcgagttccctttcacattcggcagcggcactaagctggagatcaagggtggcggtggctcgggcggtggtgggtcgggtggcggcggatctcaagtgcagctgcagcagagcggagccgagctgatgaagcccggcgcctccatgaagatcagctgcaaggccactggctacacattcagcacatactggatcgagtgggtcaagcagagacccggccacggactggagtggatcggagagaatctgcccggaaggcacatcactaactacaacgagaagttcaagggcaaggccactttcacagccgacactagcagcaacactgcctacatgcagctcagctctctgacaagcgaagatagcgccgtgtactactgtgctaggggaaggggcacttactacttcgattactggggccaaggcactccactgactgtgtccagc

SEQ ID NO: 137 - DNA, 735 bp, Artificial Sequence
  Nucleic acid molecule
caagtgcagctgcagcagagcggagccgagctgatgaagcccggcgcctccatgaagatcagctgcaaggccactggctacacattcagcacatactggatcgagtgggtcaagcagagacccggccacggactggagtggatcggagagaatctgcccggaaggcacatcactaactacaacgagaagttcaagggcaaggccactttcacagccgacactagcagcaacactgcctacatgcagctcagctctctgacaagcgaagatagcgccgtgtactactgtgctaggggaaggggcacttactacttcgattactggggccaaggcactccactgactgtgtccagcggtggcggtggctcgggcggtggtgggtcgggtggcggcggatctgatatcgtcatgactcaagccgccttcagcactccagtcactctcggcacaagcgccagcatcagctgtaggtccagccagtctctgctgcacagcaacggcatcacttatctgtactggtatctgcagaagcccggccaaagcccacagctgctgatctaccagatgagcaatctggccagcggcgtgccagatagattcagcagcagcggaagcggaacagacttcacactgaggatctctagggtggaagccgaagatgtgggcgtctattactgcgcccagaacctcgagttccctttcacattcggcagcggcactaagctggagatcaag

SEQ ID NO: 138 - DNA, 726 bp, Artificial Sequence
  Nucleic acid molecule
cagatcgtgctgacacagagcccagctctgatgagcgccagccccggcgagaaggtcacaatgacttgcagcgccacatccagcgtgagctacatctactggtaccagcagaagcctaggagcagccctaagccttggatctacctcacaagcaatctggccagcggagtgccagctaggttcagcggaagcggcagcggcacaagctactctctgacaatctccagcatggaagccgaagatgccgccacttactactgccagcagtggagcagcaatccacctacattcggaggcggcactaagctggagatcaagggtggcggtggctcgggcggtggtgggtcgggtggcggcggatctgaggtgcagctccaacagagcggaccagagctggtgaaacccggcgccagcatgaagatcagctgtaaggcctccggctacagcttcactggctacactatgaactgggtgaagcagtcccacggcaagaatctggagtggatcggactgatcaacccatacaacggcggcacaaactacaaccagaagttcaagggcaaggccactctgacagtcgataagagctccagcactgcctacatggagctgctgagcctcactagcgaggacagcgctgtgtactactgtgccttctcctactacggctctaggggcttctacttcgattactggggccaaggcacaacactgacagtgtccagc

SEQ ID NO: 139 - DNA, 726 bp, Artificial Sequence
  Nucleic acid molecule
gaggtgcagctccaacagagcggaccagagctggtgaaacccggcgccagcatgaagatcagctgtaaggcctccggctacagcttcactggctacactatgaactgggtgaagcagtcccacggcaagaatctggagtggatcggactgatcaacccatacaacggcggcacaaactacaaccagaagttcaagggcaaggccactctgacagtcgataagagctccagcactgcctacatggagctgctgagcctcactagcgaggacagcgctgtgtactactgtgccttctcctactacggctctaggggcttctacttcgattactggggccaaggcacaacactgacagtgtccagcggtggcggtggctcgggcggtggtgggtcgggtggcggcggatctcagatcgtgctgacacagagcccagctctgatgagcgccagccccggcgagaaggtcacaatgacttgcagcgccacatccagcgtgagctacatctactggtaccagcagaagcctaggagcagccctaagccttggatctacctcacaagcaatctggccagcggagtgccagctaggttcagcggaagcggcagcggcacaagctactctctgacaatctccagcatggaagccgaagatgccgccacttactactgccagcagtggagcagcaatccacctacattcggaggcggcactaagctggagatcaag

SEQ ID NO: 140 - DNA, 729 bp, Artificial Sequence
  Nucleic acid molecule
gatatcgtgatgactcagtccccagccatcatgtccgccagccccggcgagaaggtgactatgacttgctccgccagcagcagcgtgagctacatgcactggtaccagcagaagagcggcacatccccaaagaggtggatctacgacacaagcaagctggccagcggcgtgccagccagattcagcggctccggcagcggaacaagctactctctgactatcagcagcatggagacagaggacgctgccacttactactgccagcagtggagcagcaatcctccaactttcggcggaggcactaagctggagctgaagggtggcggtggctcgggcggtggtgggtcgggtggcggcggatctgaagtgcagctgcaacagagcggcccagagctcgtgaagcccggcgccagcatgaagatcagctgcaaggccagcggctacagcttcactggctacacaatgaactgggtcaagcagagccacggaaaaaatctggagtggatcggactgatcaacccttacaacggcggcacaaggtacaatcagaagttcaagggcaaggccactctcactgtggataagagcagcagcactgcctacatggagctgctgtctctgacaagcgaggatagcgccgtgtactactgcgccagctcctcctataggaacgacggcaactggtacttcgatgtgtggggcgccggcactactgtgacagtgagctcc

SEQ ID NO: 141 - DNA, 729 bp, Artificial Sequence
  Nucleic acid molecule
gaagtgcagctgcaacagagcggcccagagctcgtgaagcccggcgccagcatgaagatcagctgcaaggccagcggctacagcttcactggctacacaatgaactgggtcaagcagagccacggaaaaaatctggagtggatcggactgatcaacccttacaacggcggcacaaggtacaatcagaagttcaagggcaaggccactctcactgtggataagagcagcagcactgcctacatggagctgctgtctctgacaagcgaggatagcgccgtgtactactgcgccagctcctcctataggaacgacggcaactggtacttcgatgtgtggggcgccggcactactgtgacagtgagctccggtggcggtggctcgggcggtggtgggtcgggtggcggcggatctgatatcgtgatgactcagtccccagccatcatgtccgccagccccggcgagaaggtgactatgacttgctccgccagcagcagcgtgagctacatgcactggtaccagcagaagagcggcacatccccaaagaggtggatctacgacacaagcaagctggccagcggcgtgccagccagattcagcggctccggcagcggaacaagctactctctgactatcagcagcatggagacagaggacgctgccacttactactgccagcagtggagcagcaatcctccaactttcggcggaggcactaagctggagctgaag

SEQ ID NO: 142 - DNA, 726 bp, Artificial Sequence
  Nucleic acid molecule
cagattgtgctgtcccagtccccagccattctgagcgccagccccggcgagaaggtgactatgacttgtagggccacaagcagcgtgagctacatgtactggtaccagcagaagcccggcagcagccctaagccttggatctacgccacaagcaatctggccagcggcgtcccagctagatttagcggcagcggatccggcactagctattctctgactatctctagggtcgaggccgaagatgccgccacatactactgccagcagtggtcctccaatcctccaacattcggcggcggaactaagctggagaagaagggtggcggtggctcgggcggtggtgggtcgggtggcggcggatctgaagtgcagctgcagcagagcggaccagagctggtcaagcccggcgccagcatgaagatcagctgtaaggccagcggctacagcatcactggctacactatgaactgggtgaagcagagccacggcaagaacctcgagtggattggcctcgtgaacccatacaacggcggcacttcctacaaccagaagttcaaaggcaaggccacactcacagtcgataagtccagctccacagcctacatggagctgctgtctctgaagagcgaggatagcgctgtctactactgtgccatcagcagatacggcagcgagagctggtacttcgacgtgtggggcgccggcacaacagtgacagtgagcagc

SEQ ID NO: 143 - DNA, 726 bp, Artificial Sequence
  Nucleic acid molecule
gaagtgcagctgcagcagagcggaccagagctggtcaagcccggcgccagcatgaagatcagctgtaaggccagcggctacagcatcactggctacactatgaactgggtgaagcagagccacggcaagaacctcgagtggattggcctcgtgaacccatacaacggcggcacttcctacaaccagaagttcaaaggcaaggccacactcacagtcgataagtccagctccacagcctacatggagctgctgtctctgaagagcgaggatagcgctgtctactactgtgccatcagcagatacggcagcgagagctggtacttcgacgtgtggggcgccggcacaacagtgacagtgagcagcggtggcggtggctcgggcggtggtgggtcgggtggcggcggatctcagattgtgctgtcccagtccccagccattctgagcgccagccccggcgagaaggtgactatgacttgtagggccacaagcagcgtgagctacatgtactggtaccagcagaagcccggcagcagccctaagccttggatctacgccacaagcaatctggccagcggcgtcccagctagatttagcggcagcggatccggcactagctattctctgactatctctagggtcgaggccgaagatgccgccacatactactgccagcagtggtcctccaatcctccaacattcggcggcggaactaagctggagaagaag

SEQ ID NO: 144 - DNA, 732 bp, Artificial Sequence
  Nucleic acid molecule
gatatcgtgctgactcagagcccagcctctctggtggtgtctctgggacagagggccacaatcagctgtaggacttccaagagcgtgagcagctccgcctacagctacatgcactggtaccagcagaagcccggccagcctcctaaggtgctgatctatctggccagcaatctggagagcggcgtcccagctagattcagcggctccggaagcggcactgacttcactctgaacatccacccagtggaagaggaggatgccgccacatactactgccagcactctagggagctgcctttcacatttggcagcggaactaagctggagatcaagggtggcggtggctcgggcggtggtgggtcgggtggcggcggatctcaagtgcatctgcagcagagcggcgctgagctgatgaagcccggcgccagcgtgaagattagctgcaaggccactggctacacattcaacatctactggatcgactgggtgaagcagaggcccggccacggactggaatggatcggcgaaattctgcccggcagcggcaacactcactacaacgagaacttcaagggcaaggccacaatgacagccgacacaagctccaacactgcttacatgcagctgacttctctgactagcgaggacagcgccgtgtactattgcgctaggacagacggaaggggctacttcgattactggggccaaggcactacactcacagtgagcagc

SEQ ID NO: 145 - DNA, 732 bp, Artificial Sequence
  Nucleic acid molecule
caagtgcatctgcagcagagcggcgctgagctgatgaagcccggcgccagcgtgaagattagctgcaaggccactggctacacattcaacatctactggatcgactgggtgaagcagaggcccggccacggactggaatggatcggcgaaattctgcccggcagcggcaacactcactacaacgagaacttcaagggcaaggccacaatgacagccgacacaagctccaacactgcttacatgcagctgacttctctgactagcgaggacagcgccgtgtactattgcgctaggacagacggaaggggctacttcgattactggggccaaggcactacactcacagtgagcagcggtggcggtggctcgggcggtggtgggtcgggtggcggcggatctgatatcgtgctgactcagagcccagcctctctggtggtgtctctgggacagagggccacaatcagctgtaggacttccaagagcgtgagcagctccgcctacagctacatgcactggtaccagcagaagcccggccagcctcctaaggtgctgatctatctggccagcaatctggagagcggcgtcccagctagattcagcggctccggaagcggcactgacttcactctgaacatccacccagtggaagaggaggatgccgccacatactactgccagcactctagggagctgcctttcacatttggcagcggaactaagctggagatcaag

SEQ ID NO: 146 - DNA, 714 bp, Artificial Sequence
  Nucleic acid molecule
caaattgttctcacacagagccctcctatcatgagcgccagccccggcgagaaggtgactatgacttgttccgccagcagcaacatcagctacatgcactggtaccagcagaagtccggcacaagcccaaagaggtggatctacgacacaagcaagctggccagcggcgtgccagccagatttagcggctccggaagcggcactagctactctctgacaatcagcagcatggaagccgaggacgccgctacatactactgccagcagtggagctccgtcccactgactttcggcgctggcactaagctggagatcaagggtggcggtggctcgggcggtggtgggtcgggtggcggcggatctcaggttcagctgcagcagtccggcgctgagctcatgaagcccggcgccagcgtgaagatcagctgcaaggccactggctacacattcagcagctactggatcgagtgggtgaagcagagacccggccacggactggaatggctgggagagtttctgcctagaagcggcaagacaaactacaacgaggagtttaggggcaaggctacattcactgccgacacatccagcaacactgcctacatgcagctgagcagcctcacaagcgaggattccgccgtctactactgtgctaggactgatcctccttacttcggagtgtggggcgctggcacaatggtggctgtgagcagc

SEQ ID NO: 147 - DNA, 714 bp, Artificial Sequence
  Nucleic acid molecule
caggttcagctgcagcagtccggcgctgagctcatgaagcccggcgccagcgtgaagatcagctgcaaggccactggctacacattcagcagctactggatcgagtgggtgaagcagagacccggccacggactggaatggctgggagagtttctgcctagaagcggcaagacaaactacaacgaggagtttaggggcaaggctacattcactgccgacacatccagcaacactgcctacatgcagctgagcagcctcacaagcgaggattccgccgtctactactgtgctaggactgatcctccttacttcggagtgtggggcgctggcacaatggtggctgtgagcagcggtggcggtggctcgggcggtggtgggtcgggtggcggcggatctcaaattgttctcacacagagccctcctatcatgagcgccagccccggcgagaaggtgactatgacttgttccgccagcagcaacatcagctacatgcactggtaccagcagaagtccggcacaagcccaaagaggtggatctacgacacaagcaagctggccagcggcgtgccagccagatttagcggctccggaagcggcactagctactctctgacaatcagcagcatggaagccgaggacgccgctacatactactgccagcagtggagctccgtcccactgactttcggcgctggcactaagctggagatcaag

SEQ ID NO: 148 - protein, 327 aa, Artificial Sequence
  Constant region of human IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 149 - protein, 214 aa, Artificial Sequence
  Light chain of tanezumab
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 447 aa, Artificial Sequence
  Heavy chain of tanezumab
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 151 - protein, 213 aa, Artificial Sequence
  Light chain of MNAC13
DIVLTQSPSSLSASVGDRVTITCSASSSVSYMHWYQQKPGQAPKLLIYTTSNLASGVPSRFSGSGSGTDYTLTISSLQPEDVATYYCHQWSSYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 449 aa, Artificial Sequence
  Heavy chain of MNAC13
EVQLLESGGGLVQPGGSLRLSCAASGFTFSTYTMSWARQAPGKGLEWVAYISKGGGSTYYPDTVKGRFTISRDNSKNTLYLQMNSLRAEDSAVYYCARGAMFGNDFFFPMDRWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

(19) United States
(12) Patent Application Publication
(10) Pub. No.: US 2022/0204630 A1
(43) Pub. Date: Jun. 30, 2022

(54) ANTI-TRKA ANTIBODIES AND USES THEREOF

(71) Applicant: SUNSHINE LAKE PHARMA CO., LTD., Dongguan, Guangdong (CN)

(72) Inventors: Chao CHEN, Dongguan (CN); Zhiheng REN, Dongguan (CN); Zhuandi HE, Dongguan (CN); Jielian LU, Dongguan (CN); Shushan LIN, Dongguan (CN); Tingting YU, Dongguan (CN); Xiling WEI, Dongguan (CN); Xufang WANG, Dongguan (CN); Le XU, Dongguan (CN); Junji DONG, Dongguan (CN); Xiang LI, Dongguan (CN); Kuo ZHANG, Dongguan (CN); Xueyao YANG, Dongguan (CN); Linfeng GUO, Dongguan (CN); Xiaoping LI, Dongguan (CN); Xiaofeng CHEN, Dongguan (CN); Wenjia LI, Dongguan (CN)

(73) Assignee: SUNSHINE LAKE PHARMA CO., LTD., Dongguan, Guangdong (CN)

(21) Appl. No.: 17/604,728

(22) PCT Filed: May 28, 2020
(86) PCT No.: PCT/CN2020/092766

§ 371 Date: Oct. 18, 2021

(30) Foreign Application Priority Data
May 30, 2019 (CN) 201910464016.0

(51) Int. Cl. C07K 16/28; A61P 25/04; G01N 33/68

(52) U.S. Cl. CPC C07K 16/2863; A61K 2039/505; G01N 33/6893; A61P 25/04

(57) ABSTRACT

An antibody or an antigen-binding fragment thereof is capable of specifically recognizing TrkA and uses thereof. The antibody contains a CDR sequence selected from at least one of the following or an amino acid sequence having at least 95% identity with it: heavy chain variable region CDR sequences: SEQ ID NO: 1˜27, light chain variable region CDR sequences: SEQ IN NO: 28˜54. The above antibody can specifically targeted-bind to the TrkA receptor and block the binding of NGF to TrkA.

### CROSS-REFERENCE TO RELATED APPLICATION

This application claims the priority and benefits of Chinese Patent Application No. 201910464016.0, filed with the State Intellectual Property Office of China on May 30, 2019, which is incorporated herein by reference in its entirety.

### FIELD OF THE INVENTION

The present invention relates to the field of biotechnology. Specifically, the present invention relates to anti-TrkA antibodies and uses thereof. More specifically, the present invention relates to an antibody or an antigen-binding fragment thereof, a nucleic acid molecule, an expression vector, a recombinant cell, a pharmaceutical composition, pharmaceutical uses, a kit for detecting TrkA, and a mouse B cell capable of specifically recognize TrkA.

### BACKGROUND OF THE INVENTION

At present, non-opioid analgesics are mainly used clinically for mild to moderate pain, such as non-steroidal anti-inflammatory drugs (NSAIDs); opioid analgesics are mainly used for moderate to severe pain. However, NSAIDs have a “capped effect”, and opioids only can effectively relieve less than 30% of non-tumor chronic pain, and 20% of patients with cancer pain have opioid resistance. In addition, NSAIDs have hidden dangers of gastrointestinal and cardiovascular safety, especially during long-term medication. For opioid analgesics, years of drug improvement experiments have failed to effectively reduce its addiction and many other side effects, and patients expect new safer and more effective drugs.

Nerve growth factor (NGF) is involved in the pathophysiological process of pain. It mainly activates the NGF/TrKA signaling pathway by binding to high-affinity tyrosine-nase (TrkA) receptors, affects the release of inflammatory mediators, the opening of ion channels, and promotes the growth of nerve fibers, thereby participating in the occurrence, conduction and sensitization of pain. Studies have shown that blocking NGF-TrkA signaling pathway can effectively relieve pain and hyperalgesia, and NGF-TrkA signaling pathway is an effective target for the development of new analgesics. For NGF-TrkA target analgesics, the drugs currently under development are mainly divided into two categories: large molecule anti-NGF monoclonal antibodies and small molecule TrkA kinase inhibitors. Since the TrKA receptor is not the only receptor of NGF, NGF also has a low affinity receptor P75, and the combination of NGF and P75 is important for the function of the nervous system and damage repair. Large molecular anti-NGF monoclonal antibodies have great safety risks as a new type of analgesics. From the current clinical data, the most common toxic and side effects include bone tissue necrosis (potential), sensory abnormalities, etc. However, there are more problems with small molecule TrkA kinase inhibitors. First, small molecule drugs generally have short half-life, frequent administration, and poor patient compliance. Secondly, small molecule TrkA kinase inhibitors are receptor tyrosine kinase inhibitors, and there are many receptor tyrosine kinases in the body. Therefore, the specificity and selectivity of drugs for specific targets are difficult to guarantee, and there are many adverse reactions and large toxic and side effects.

Therefore, analgesics targeting NGF-TrkA targets still need to be continuously developed and improved by scientific researchers.

### SUMMARY OF THE INVENTION

This application is based on the inventors' discovery of the following issues and facts:

the NGF-TrkA signaling pathway is an effective target for the development of new analgesics. If TrkA monoclonal antibodies selectively target the TrkA receptor, it can not only block the activation of the TrkA signaling pathway by NGF, effectively inhibit the transmission of pain signals, but also not cause many neurotoxicities caused by the inhibition of NGF receptors and unpredictable toxic and side effects such as using of anti-NGF antibodies to over-neutralize the bone and joint necrosis caused by NGF. However, because the TrkA molecule is a receptor membrane protein, it is difficult to screen the blocking anti-TrkA monoclonal antibodies. In addition, designing the blocking TrkA receptor antibodies has safety risks due to antibody-mediated immune responses. Therefore, it is difficult to design and develop monoclonal antibodies against TrkA.

However, the inventors of this application have successfully screened a new type of anti-TrkA monoclonal antibody with long-acting analgesic effect. At the same time, the inventors have effectively avoided the risk of immunotoxicity by selecting specific Fc subtypes and modifying them. Specifically, in this study, a series of high-affinity anti-TrkA monoclonal antibodies that block the binding of NGF and TrkA were screened by hybridoma technology. The constant region of mouse anti-TrkA monoclonal antibody was humanized and the variable region of the mouse anti-TrkA monoclonal antibody was retained. A series of anti-TrkA chimeric monoclonal antibodies were obtained. The inventors have discovered that these chimeric antibody candidates obtained in this application can not only specifically targeted-bind to the TrkA receptor, block the binding of NGF and TrkA, effectively inhibit pain, but also have the characteristics of low immunotoxicity and almost no antibody-dependent cell-mediated cytotoxicity (ADCC).

In a first aspect of the present invention, the present invention provides an antibody or an antigen-binding fragment thereof capable of specifically recognizing TrkA. According to the embodiment of the present invention, the antibody contains a CDR sequence selected from at least one of the following or an amino acid sequence having at least 95% identity with it: heavy chain variable region CDR sequences: SEQ ID NO: 1˜27, light chain variable region CDR sequences: SEQ IN NO: 28˜54.

(SEQ ID NO: 1)
VYSFTAYT.
(SEQ ID NO: 2)
INPHNGGT.
(SEQ ID NO: 3)
AISRYGSSSFYFDV.
(SEQ ID NO: 4)
GYAFTNYW.
(SEQ ID NO: 5)
FYPRTGNT.
(SEQ ID NO: 6)
ARAGTGFDY.
(SEQ ID NO: 7)
GYRFSSYW.
(SEQ ID NO: 8)
ILPGRGII.
(SEQ ID NO: 9)
ARTDPPYFGV.
(SEQ ID NO: 10)
GYTFSTYW.
(SEQ ID NO: 11)
NLPGRHIT.
(SEQ ID NO: 12)
ARGRGTYYFDY.
(SEQ ID NO: 13)
GYSFTGYT.
(SEQ ID NO: 14)
INPYNGGT.
(SEQ ID NO: 15)
AFSYYGSRGFYFDY.
(SEQ ID NO: 16)
GYSFTGYT.
(SEQ ID NO: 17)
INPYNGGT.
(SEQ ID NO: 18)
ASSSYRNDGNWYFDV.
(SEQ ID NO: 19)
GYSITGYT.
(SEQ ID NO: 20)
VNPYNGGT.
(SEQ ID NO: 21)
AISRYGSESWYFDV.
(SEQ ID NO: 22)
GYTFNIYW.
(SEQ ID NO: 23)
ILPGSGNT.
(SEQ ID NO: 24)
ARTDGRGYFDY.
(SEQ ID NO: 25)
GYTFSSYW.
(SEQ ID NO: 26)
FLPRSGKT.
(SEQ ID NO: 27)
ARTDPPYFGV.
(SEQ ID NO: 28)
SSISY.
(SEQ ID NO: 29)
ATS.
(SEQ ID NO: 30)
QQWSSNPPT.
(SEQ ID NO: 31)
ENVGGY.
(SEQ ID NO: 32)
GAS.
(SEQ ID NO: 33)
GQNYIYPFT.
(SEQ ID NO: 34)
TGAVTISNY.
(SEQ ID NO: 35)
GTN.
(SEQ ID NO: 36)
VLWYSNHWV.
(SEQ ID NO: 37)
QSLLHSNGITY.
(SEQ ID NO: 38)
QMS.
(SEQ ID NO: 39)
AQNLEFPFT.
(SEQ ID NO: 40)
SSVSY.
(SEQ ID NO: 41)
LTS.
(SEQ ID NO: 42)
QQWSSNPPT.
(SEQ ID NO: 43)
SSVSY.
(SEQ ID NO: 44)
DTS.
(SEQ ID NO: 45)
QQWSSNPPT.
(SEQ ID NO: 46)
SSVSY.
(SEQ ID NO: 47)
ATS.
(SEQ ID NO: 48)
QQWSSNPPT.
(SEQ ID NO: 49)
KSVSSSAYSY.
(SEQ ID NO: 50)
LAS.
(SEQ ID NO: 51)
QHSRELPFT.
(SEQ ID NO: 52)
SNISY.
(SEQ ID NO: 53)
DTS.
(SEQ ID NO: 54)
QQWSSVPLT.

The above antibody according to the embodiment of the present invention can specifically targeted-bind to the TrkA receptor and block the binding of NGF and TrkA.

According to the embodiment of the present invention, the antibody or antigen-binding fragment may further include at least one of the following additional technical features:

according to the embodiment of the invention, the antibody includes:

heavy chain variable region CDR1, CDR2, CDR3 sequences as shown in SEQ ID NO: 1, 2 and 3, or an amino acid sequence having at least 95% identity with SEQ ID NO: 1, 2 and 3, respectively; or

heavy chain variable region CDR1, CDR2, CDR3 sequences as shown in SEQ ID NO: 4, 5 and 6, or an amino acid sequence having at least 95% identity with SEQ ID NO: 4, 5 and 6, respectively; or

heavy chain variable region CDR1, CDR2, CDR3 sequences as shown in SEQ ID NO: 7, 8 and 9, or an amino acid sequence having at least 95% identity with SEQ ID NO: 7, 8 and 9, respectively; or

heavy chain variable region CDR1, CDR2, CDR3 sequences as shown in SEQ ID NO: 10, 11 and 12, or an amino acid sequence having at least 95% identity with SEQ ID NO: 10, 11 and 12, respectively; or

heavy chain variable region CDR1, CDR2, CDR3 sequences as shown in SEQ ID NO: 13, 14 and 15, or an amino acid sequence having at least 95% identity with SEQ ID NO: 13, 14 and 15, respectively; or

heavy chain variable region CDR1, CDR2, CDR3 sequences as shown in SEQ ID NO: 16, 17 and 18, or an amino acid sequence having at least 95% identity with SEQ ID NO: 16, 17 and 18, respectively; or

heavy chain variable region CDR1, CDR2, CDR3 sequences as shown in SEQ ID NO: 19, 20 and 21, or an amino acid sequence having at least 95% identity with SEQ ID NO: 19, 20 and 21, respectively; or

heavy chain variable region CDR1, CDR2, CDR3 sequences as shown in SEQ ID NO: 22, 23 and 24, or an amino acid sequence having at least 95% identity with SEQ ID NO: 22, 23 and 24, respectively; or

heavy chain variable region CDR1, CDR2, CDR3 sequences as shown in SEQ ID NO: 25, 26 and 27, or an amino acid sequence having at least 95% identity with SEQ ID NO: 25, 26 and 27, respectively.

According to the embodiment of the invention, the antibody includes:

light chain variable region CDR1, CDR2, CDR3 sequences as shown in SEQ ID NO: 28, 29 and 30, or an amino acid sequence having at least 95% identity with SEQ ID NO: 28, 29 and 30, respectively; or

light chain variable region CDR1, CDR2, CDR3 sequences as shown in SEQ ID NO: 31, 32 and 33, or an amino acid sequence having at least 95% identity with SEQ ID NO: 31, 32 and 33, respectively; or

light chain variable region CDR1, CDR2, CDR3 sequences as shown in SEQ ID NO: 34, 35 and 36, or an amino acid sequence having at least 95% identity with SEQ ID NO: 34, 35 and 36, respectively; or

light chain variable region CDR1, CDR2, CDR3 sequences as shown in SEQ ID NO: 37, 38 and 39, or an amino acid sequence having at least 95% identity with SEQ ID NO: 37, 38 and 39, respectively; or

light chain variable region CDR1, CDR2, CDR3 sequences as shown in SEQ ID NO: 40, 41 and 42, or an amino acid sequence having at least 95% identity with SEQ ID NO: 40, 41 and 42, respectively; or

light chain variable region CDR1, CDR2, CDR3 sequences as shown in SEQ ID NO: 43, 44 and 45, or an amino acid sequence having at least 95% identity with SEQ ID NO: 43, 44 and 45, respectively; or

light chain variable region CDR1, CDR2, CDR3 sequences as shown in SEQ ID NO: 46, 47 and 48, or an amino acid sequence having at least 95% identity with SEQ ID NO: 46, 47 and 48, respectively; or

light chain variable region CDR1, CDR2, CDR3 sequences as shown in SEQ ID NO: 49, 50 and 51, or an amino acid sequence having at least 95% identity with SEQ ID NO: 49, 50 and 51, respectively; or

light chain variable region CDR1, CDR2, CDR3 sequences as shown in SEQ ID NO: 52, 53 and 54, or an amino acid sequence having at least 95% identity with SEQ ID NO: 52, 53 and 54, respectively.

According to the embodiment of the invention, the antibody or antigen-binding fragment thereof specifically recognizes the extracellular region of TrkA.

According to the embodiment of the invention, the antibody contains at least one of a heavy chain framework region sequence and a light chain framework region sequence, and at least a part of at least one of the heavy chain framework region sequence and the light chain framework region sequence is derived from at least one of a murine antibody, a human antibody, a primate antibody, or a mutant thereof.

According to the embodiment of the invention, the antibody has a heavy chain variable region of the amino acid sequence shown in any one of SEQ ID NO: 55˜63.

(SEQ ID NO: 55)
EVLLQQSGPELVKPGASMKISCKASVYSFTAYTMNWVKQSHGKNLEWIG
LINPHNGGTRYNQKFKGKATLTLDKSSSTAYMDLLSLTSEDSAVYYCAI
SRYGSSSFYFDVWGAGTTVAVSS.
(SEQ ID NO: 56)
QVQLQQSGAELVRPGTSVKISCKASGYAFTNYWLGWMKQRPGHGLEWIG
DFYPRTGNTFYNENFKGKVTLTADKSSNTAYMQLSSLTSEDSAVYLCAR
AGTGFDYWGQGTTLTVSS.
(SEQ ID NO: 57)
QVQLQQSGAELMKPGASVKISCKTTGYRFSSYWIEWVKQRPGHGLEWLG
EILPGRGIINYNENFRGKATFTADTSSNTAYVQLSSLTSEDSAVYFCAR
TDPPYFGVWGAGTTVTVSS.
(SEQ ID NO: 58)
QVQLQQSGAELMKPGASMKISCKATGYTFSTYWIEWVKQRPGHGLEWIG
ENLPGRHITNYNEKFKGKATFTADTSSNTAYMQLSSLTSEDSAVYYCAR
GRGTYYFDYWGQGTPLTVSS.
(SEQ ID NO: 59)
EVQLQQSGPELVKPGASMKISCKASGYSFTGYTMNWVKQSHGKNLEWIG
LINPYNGGTNYNQKFKGKATLTVDKSSSTAYMELLSLTSEDSAVYYCAF
SYYGSRGFYFDYWGQGTTLTVSS.
(SEQ ID NO: 60)
EVQLQQSGPELVKPGASMKISCKASGYSFTGYTMNWVKQSHGKNLEWIG
LINPYNGGTRYNQKFKGKATLTVDKSSSTAYMELLSLTSEDSAVYYCAS
SSYRNDGNWYFDVWGAGTTVTVSS.
(SEQ ID NO: 61)
EVQLQQSGPELVKPGASMKISCKASGYSITGYTMNWVKQSHGKNLEWIG
LVNPYNGGTSYNQKFKGKATLTVDKSSSTAYMELLSLKSEDSAVYYCAI
SRYGSESWYFDVWGAGTTVTVSS.
(SEQ ID NO: 62)
QVHLQQSGAELMKPGASVKISCKATGYTFNIYWIDWVKQRPGHGLEWIG
EILPGSGNTHYNENFKGKATMTADTSSNTAYMQLTSLTSEDSAVYYCAR
TDGRGYFDYWGQGTTLTVSS.
(SEQ ID NO: 63)
QVQLQQSGAELMKPGASVKISCKATGYTFSSYWIEWVKQRPGHGLEWLG
EFLPRSGKTNYNEEFRGKATFTADTSSNTAYMQLSSLTSEDSAVYYCAR
TDPPYFGVWGAGTMVAVSS.

According to the embodiment of the present invention, the antibody has a light chain variable region of an amino acid sequence shown in any one of SEQ ID NO: 64˜72.

(SEQ ID NO: 64)
QIVLSQSPAILSASPGEKVTMTCRASSSISYMHWYQQKPGSSPKPWISA
TSNLASGVPARFSGSGSGTSYSLTISGVEAEDAATYYCQQWSSNPPTFG
GGTNLEIK.
(SEQ ID NO: 65)
SIVMTQSPKSMSMSVGERVTLSCKASENVGGYVSWYQQKPDQSPKLLIY
GASSRHTGVPDRFTGSGSETDFTLTISSVQAEDLAAYHCGQNYIYPFTF
GGGTKLEIK.
(SEQ ID NO: 66)
QAVVTQESALTTSPGETVTLTCRSSTGAVTISNYANWVQEKPDHLFTGL
IGGTNNRPPGVPARFSGSLIGDKAALTITGAQTEDEAIYFCVLWYSNHW
VFGGGTKLTVL.
(SEQ ID NO: 67)
DIVMTQAAFSTPVTLGTSASISCRSSQSLLHSNGITYLYWYLQKPGQSP
QLLIYQMSNLASGVPDRFSSSGSGTDFTLRISRVEAEDVGVYYCAQNLE
FPFTFGSGTKLEIK.
(SEQ ID NO: 68)
QIVLTQSPALMSASPGEKVTMTCSATSSVSYIYWYQQKPRSSPKPWIYL
TSNLASGVPARFSGSGSGTSYSLTISSMEAEDAATYYCQQWSSNPPTFG
GGTKLEIK.
(SEQ ID NO: 69)
DIVMTQSPAIMSASPGEKVTMTCSASSSVSYMHWYQQKSGTSPKRWIYD
TSKLASGVPARFSGSGSGTSYSLTISSMETEDAATYYCQQWSSNPPTFG
GGTKLELK.
(SEQ ID NO: 70)
QIVLSQSPAILSASPGEKVTMTCRATSSVSYMYWYQQKPGSSPKPWIYA
TSNLASGVPARFSGSGSGTSYSLTISRVEAEDAATYYCQQWSSNPPTFG
GGTKLEKK.
(SEQ ID NO: 71)
DIVLTQSPASLVVSLGQRATISCRTSKSVSSSAYSYMHWYQQKPGQPPK
VLIYLASNLESGVPARFSGSGSGTDFTLNIHPVEEEDAATYYCQHSREL
PFTFGSGTKLEIK.
(SEQ ID NO: 72)
QIVLTQSPPIMSASPGEKVTMTCSASSNISYMHWYQQKSGTSPKRWIYD
TSKLASGVPARFSGSGSGTSYSLTISSMEAEDAATYYCQQWSSVPLTFG
AGTKLEIK.

According to the embodiment of the present invention, the antibody contains at least one of a heavy chain constant region and a light chain constant region, and at least a part of at least one of the heavy chain constant region and the light chain constant region is derived from at least one of a murine antibody, a human antibody, a primate antibody, or a mutant thereof.

According to the embodiment of the present invention, the light chain constant region and heavy chain constant region of the antibody are both derived from a human IgG antibody or a mutant thereof. Thus, the immunogenicity of the antibody can be effectively reduced.

According to the embodiment of the present invention, the light chain constant region and heavy chain constant region of the antibody are both derived from a human IgG4.

According to the embodiment of the present invention, the Fc region of the antibody has S10P, F16A, L17A, R191K mutations and 229 K deletion mutations compared to human IgG4 wild type Fc. Wherein, the positioning of the above amino acid position is based on the amino acid sequence of the human IgG4 wild type Fc sequence shown in SEQ ID NO: 73. For example, S10P refers to the mutation of S at position 10 of the amino acid sequence shown in SEQ ID NO: 75 to P, and so on. The inventors found that after the Fc region of the antibody has the above mutations and deletions, the safety and stability of the antibody can be significantly improved, and the half-life of the antibody in the body is also significantly extended.

(SEQ ID NO: 73)
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFScSVMHEALHNHYTQKSLSLSLGK.

According to the embodiment of the present invention, the full-length sequence of the constant region of the antibody is shown in SEQ ID NO: 74 or 75.

(SEQ ID NO: 74)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEAAGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSKLTVDK
SRWQEGNVFSCSVMHEALHNHYTQKSLSLSLG.
(SEQ ID NO: 75)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

Wherein the full-length sequence of the constant region of the antibody shown in the above SEQ ID NO: 74 includes the IgG4 heavy chain constant region and the Fc region. Wherein the sequence of the IgG4 heavy chain constant region is ASTKGPSVFPLAPCSRSTSESTAALGCLVKDYFPEPVTVSWNSGALTSGVHTFPAVLQSSGLY SLSSVVTVPSSSLGTKTYTCNVDHKPSNTKVDKRV, the sequence of the Fc region is ESKYGPPCPPCPAPEAAGGPSVFLFPPKPKDTLMISRTPEVTCVVVDVSQEDPEVQFNWYVD GVEVHNAKTKPREEQFNSTYRVVSVLTVLHQDWLNGKEYKCKVSNKGLPSSIEKTISKAK GQPREPQVYTLPPSQEEMTKNQVSLTCLVKGFYPSDIAVEWESNGQPENNYKTTPPVLDSD GSFFLYSKLTVDKSRWQEGNVFSCSVMHEALHNHYTQKSLSLSLG. The full-length sequence of the constant region of the antibody shown in the above SEQ ID NO: 75 is the IgG4 light chain constant region.

According to the embodiment of the present invention, the antibody has a heavy chain of the amino acid sequence shown in any one of SEQ ID NO: 76˜84 and a light chain of the amino acid sequence shown in any one of SEQ ID NO: 85˜93.

(SEQ ID NO: 76)
EVLLQQSGPELVKPGASMKISCKASVYSFTAYTMNWVKQSHGKNLEWIGLINPHN
GGTRYNQKFKGKATLTLDKSSSTAYMDLLSLTSEDSAVYYCAISRYGSSSFYFDVWGAGTT
VAVSSASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPPCPAPEAAGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSKLTVDKSRWQEGNV
FSCSVMHEALHNHYTQKSLSLSLG.
(SEQ ID NO: 85)
QIVLSQSPAILSASPGEKVTMTCRASSSISYMHWYQQKPGSSPKPWISATSNLASGV
PARFSGSGSGTSYSLTISGVEAEDAATYYCQQWSSNPPTFGGGTNLEIKRTVAAPSVFIFPPSD
EQLKSGTASVVCLLNNFYPREAKVQWKVDNALQSGNSQESVTEQDSKDSTYSLSSTLTLSK
ADYEKHKVYACEVTHQGLSSPVTKSFNRGEC.
(SEQ ID NO: 77)
QVQLQQSGAELVRPGTSVKISCKASGYAFTNYWLGWMKQRPGHGLEWIGDFYPR
TGNTFYNENFKGKVTLTADKSSNTAYMQLSSLTSEDSAVYLCARAGTGFDYWGQGTTLTVS
SASTKGPSVFPLAPCSRSTSESTAALGCLVKDYFPEPVTVSWNSGALTSGVHTFPAVLQSSGL
YSLSSVVTVPSSSLGTKTYTCNVDHKPSNTKVDKRVESKYGPPCPPCPAPEAAGGPSVFLFP
PKPKDTLMISRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQEEMTKNQVSL
TCLVKGFYPSDIAVEWESNGQPENNYKTTPPVLDSDGSFFLYSKLTVDKSRWQEGNVFSCS
VMHEALHNHYTQKSLSLSLG.
(SEQ ID NO: 86)
SIVMTQSPKSMSMSVGERVTLSCKASENVGGYVSWYQQKPDQSPKLLIYGASSRH
TGVPDRFTGSGSETDFTLTISSVQAEDLAAYHCGQNYIYPFTFGGGTKLEIKRTVAAPSVFIFP
PSDEQLKSGTASVVCLLNNFYPREAKVQWKVDNALQSGNSQESVTEQDSKDSTYSLSSTLT
LSKADYEKHKVYACEVTHQGLSSPVTKSFNRGEC.
(SEQ ID NO: 78)
QVQLQQSGAELMKPGASVKISCKTTGYRFSSYWIEWVKQRPGHGLEWLGEILPGR
GIINYNENFRGKATFTADTSSNTAYVQLSSLTSEDSAVYFCARTDPPYFGVWGAGTTVTVSS
ASTKGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPPCPAPEAAGGPSVFLFPP
KPKDTLMISRTPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSLT
CLVKGFYPSDIAVEWESNGQPENNYKTTPPVLDSDGSFFLYSKLTVDKSRWQEGNVFSCSV
MHEALHNHYTQKSLSLSLG.
(SEQ ID NO: 87)
QAVVTQESALTTSPGETVTLTCRSSTGAVTISNYANWVQEKPDHLFTGLIGGTNNRP
PGVPARFSGSLIGDKAALTITGAQTEDEAIYFCVLWYSNHWVFGGGTKLTVLRTVAAPSVFI
FPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC.
(SEQ ID NO: 79)
QVQLQQSGAELMKPGASMKISCKATGYTFSTYWIEWVKQRPGHGLEWIGENLPG
RHITNYNEKFKGKATFTADTSSNTAYMQLSSLTSEDSAVYYCARGRGTYYFDYWGQGTPLT
VSSASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEAAGGPSVFL
FPPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQV
SLTCLVKGFYPSDIAVEWESNGQPENNYKTTPPVLDSDGSFFLYSKLTVDKSRWQEGNVFSC
SVMHEALHNHYTQKSLSLSLG.
(SEQ ID NO: 88)
DIVMTQAAFSTPVTLGTSASISCRSSQSLLHSNGITYLYWYLQKPGQSPQLLIYQMS
NLASGVPDRFSSSGSGTDFTLRISRVEAEDVGVYYCAQNLEFPFTFGSGTKLEIKRTVAAPSV
FIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC.
(SEQ ID NO: 80)
EVQLQQSGPELVKPGASMKISCKASGYSFTGYTMNWVKQSHGKNLEWIGLINPYN
GGTNYNQKFKGKATLTVDKSSSTAYMELLSLTSEDSAVYYCAFSYYGSRGFYFDYWGQGT
TLTVSSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEAAGGPS
VFLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSKLTVDKSRWQEGN
VFSCSVMHEALHNHYTQKSLSLSLG.
(SEQ ID NO: 89)
QIVLTQSPALMSASPGEKVTMTCSATSSVSYIYWYQQKPRSSPKPWIYLTSNLASGV
PARFSGSGSGTSYSLTISSMEAEDAATYYCQQWSSNPPTFGGGTKLEIKRTVAAPSVFIFPPSD
EQLKSGTASVVCLLNNFYPREAKVQWKVDNALQSGNSQESVTEQDSKDSTYSLSSTLTLSK
ADYEKHKVYACEVTHQGLSSPVTKSFNRGEC.
(SEQ ID NO: 81)
EVQLQQSGPELVKPGASMKISCKASGYSFTGYTMNWVKQSHGKNLEWIGLINPYN
GGTRYNQKFKGKATLTVDKSSSTAYMELLSLTSEDSAVYYCASSSYRNDGNWYFDVWGAG
TTVTVSSASTKGPSVFPLAPCSRSTSESTAALGCLVKDYFPEPVTVSWNSGALTSGVHTFPAV
LQSSGLYSLSSVVTVPSSSLGTKTYTCNVDHKPSNTKVDKRVESKYGPPCPPCPAPEAAGGP
SVFLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSKLTVDKSRWQEGN
VFSCSVMHEALHNHYTQKSLSLSLG.
(SEQ ID NO: 90)
DIVMTQSPAMSASPGEKVTMTCSASSSVSYMHWYQQKSGTSPKRWIYDTSKLAS
GVPARFSGSGSGTSYSLTISSMETEDAATYYCQQWSSNPPTFGGGTKLELKRTVAAPSVFIFP
PSDEQLKSGTASVVCLLNNFYPREAKVQWKVDNALQSGNSQESVTEQDSKDSTYSLSSTLT
LSKADYEKHKVYACEVTHQGLSSPVTKSFNRGEC.
(SEQ ID NO: 82)
EVQLQQSGPELVKPGASMKISCKASGYSITGYTMNWVKQSHGKNLEWIGLVNPYN
GGTSYNQKFKGKATLTVDKSSSTAYMELLSLKSEDSAVYYCAISRYGSESWYFDVWGAGT
TVTVSSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEAAGGPS
VFLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSKLTVDKSRWQEGN
VFSCSVMHEALHNHYTQKSLSLSLG.
(SEQ ID NO: 91)
QIVLSQSPAILSASPGEKVTMTCRATSSVSYMYWYQQKPGSSPKPWIYATSNLASG
VPARFSGSGSGTSYSLTISRVEAEDAATYYCQQWSSNPPTFGGGTKLEKKRTVAAPSVFIFPP
SDEQLKSGTASVVCLLNNFYPREAKVQWKVDNALQSGNSQESVTEQDSKDSTYSLSSTLTL
SKADYEKHKVYACEVTHQGLSSPVTKSFNRGEC.
(SEQ ID NO: 83)
QVHLQQSGAELMKPGASVKISCKATGYTFNIYWIDWVKQRPGHGLEWIGEILPGS
GNTHYNENFKGKATMTADTSSNTAYMQLTSLTSEDSAVYYCARTDGRGYFDYWGQGTTLT
VSSASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEAAGGPSVFL
FPPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQV
SLTCLVKGFYPSDIAVEWESNGQPENNYKTTPPVLDSDGSFFLYSKLTVDKSRWQEGNVFSC
SVMHEALHNHYTQKSLSLSLG.
(SEQ ID NO: 92)
DIVLTQSPASLVVSLGQRATISCRTSKSVSSSAYSYMHWYQQKPGQPPKVLIYLASN
LESGVPARFSGSGSGTDFTLNIHPVEEEDAATYYCQHSRELPFTFGSGTKLEIKRTVAAPSVFI
FPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC.
(SEQ ID NO: 84)
QVQLQQSGAELMKPGASVKISCKATGYTFSSYWIEWVKQRPGHGLEWLGEFLPRS
GKTNYNEEFRGKATFTADTSSNTAYMQLSSLTSEDSAVYYCARTDPPYFGVWGAGTMVAVS
SASTKGPSVFPLAPCSRSTSESTAALGCLVKDYFPEPVTVSWNSGALTSGVHTFPAVLQSSGL
YSLSSVVTVPSSSLGTKTYTCNVDHKPSNTKVDKRVESKYGPPCPPCPAPEAAGGPSVFLFP
PKPKDTLMISRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQEEMTKNQVSL
TCLVKGFYPSDIAVEWESNGQPENNYKTTPPVLDSDGSFFLYSKLTVDKSRWQEGNVFSCS
VMHEALHNHYTQKSLSLSLG.
(SEQ ID NO: 93)
QIVLTQSPPIMSASPGEKVTMTCSASSNISYMHWYQQKSGTSPKRWIYDTSKLASG
VPARFSGSGSGTSYSLTISSMEAEDAATYYCQQWSSVPLTFGAGTKLEIKRTVAAPSVFIFPPS
DEQLKSGTASVVCLLNNFYPREAKVQWKVDNALQSGNSQESVTEQDSKDSTYSLSSTLTLS
KADYEKHKVYACEVTHQGLSSPVTKSFNRGEC.

Wherein, in the present application, the antibody consisting of the above SEQ ID NO: 76 and 85 is referred to as a 2008 chimeric antibody, the antibody consisting of the above SEQ ID NO: 77 and 86 is referred to as a 23E12 chimeric antibody, the antibody consisting of the above SEQ ID NO: 78 and 87 is referred to as a 27H3 chimeric antibody, the antibody consisting of the above SEQ ID NO: 79 and 88 is referred to as a 21E5 chimeric antibody, the antibody consisting of the above SEQ ID NO: 80 and 89 is referred to as a 2A5 chimeric antibody, the antibody consisting of the above SEQ ID NO: 81 and 90 is referred to as a 4H4 chimeric antibody, the antibody consisting of the above SEQ ID NO: 82 and 91 is referred to as a 22D12 chimeric antibody, the antibody consisting of the above SEQ ID NO: 83 and 92 is referred to as a 1B9 chimeric antibody, the antibody consisting of the above SEQ ID NO: 84 and 93 is referred to as a 15D4 chimeric antibody.

According to the embodiment of the present invention, the antibody is a single chain antibody fragment, a multimer antibody, a CDR grafted-antibody or a small molecule antibody.

According to the embodiment of the present invention, the antibody is a single chain antibody fragment, and the antibody has the amino acid sequence shown in SEQ ID NO: 94˜111.

(SEQ ID NO: 94)
QIVLSQSPAILSASPGEKVTMTCRASSSISYMHWYQQKPGSSPKPWISATSNLASGV
PARFSGSGSGTSYSLTISGVEAEDAATYYCQQWSSNPPTFGGGTNLEIKGGGGSGGGGSGG
GGSEVLLQQSGPELVKPGASMKISCKASVYSFTAYTMNWVKQSHGKNLEWIGLINPHNGG
TRYNQKFKGKATLTLDKSSSTAYMDLLSLTSEDSAVYYCAISRYGSSSFYFDVWGAGTTVAV
SS.
(SEQ ID NO: 95)
EVLLQQSGPELVKPGASMKISCKASVYSFTAYTMNWVKQSHGKNLEWIGLINPHN
GGTRYNQKFKGKATLTLDKSSSTAYMDLLSLTSEDSAVYYCAISRYGSSSFYFDVWGAGTT
VAVSSGGGGSGGGGSGGGGSQIVLSQSPAILSASPGEKVTMTCRASSSISYMHWYQQKPGSS
PKPWISATSNLASGVPARFSGSGSGTSYSLTISGVEAEDAATYYCQQWSSNPPTFGGGTNLEI
K.
(SEQ ID NO: 96)
SIVMTQSPKSMSMSVGERVTLSCKASENVGGYVSWYQQKPDQSPKLLIYGASSRH
TGVPDRFTGSGSETDFTLTISSVQAEDLAAYHCGQNYIYPFTFGGGTKLEIKGGGGSGGGGS
GGGGSQVQLQQSGAELVRPGTSVKISCKASGYAFTNYWLGWMKQRPGHGLEWIGDFYPR
TGNTFYNENFKGKVTLTADKSSNTAYMQLSSLTSEDSAVYLCARAGTGFDYWGQGTTLTVS
S.
(SEQ ID NO: 97)
QVQLQQSGAELVRPGTSVKISCKASGYAFTNYWLGWMKQRPGHGLEWIGDFYPR
TGNTFYNENFKGKVTLTADKSSNTAYMQLSSLTSEDSAVYLCARAGTGFDYWGQGTTLTVS
SGGGGSGGGGSGGGGSSIVMTQSPKSMSMSVGERVTLSCKASENVGGYVSWYQQKPDQS
PKLLIYGASSRHTGVPDRFTGSGSETDFTLTISSVQAEDLAAYHCGQNYIYPFTFGGGTKLEI
K.
(SEQ ID NO: 98)
QAVVTQESALTTSPGETVTLTCRSSTGAVTISNYANWVQEKPDHLFTGLIGGTNNRP
PGVPARFSGSLIGDKAALTITGAQTEDEAIYFCVLWYSNHWVFGGGTKLTVLGGGGSGGGG
SGGGGSQVQLQQSGAELMKPGASVKISCKTTGYRFSSWIEWVKQRPGHGLEWLGEILPG
RGIINYNENFRGKATFTADTSSNTAYVQLSSLTSEDSAVYFCARTDPPYFGVWGAGTTVTVS
S.
(SEQ ID NO: 99)
QVQLQQSGAELMKPGASVKISCKTTGYRFSSYWIEWVKQRPGHGLEWLGEILPGR
GIINYNENFRGKATFTADTSSNTAYVQLSSLTSEDSAVYFCARTDPPYFGVWGAGTTVTVSS
GGGGSGGGGSGGGGSQAVVTQESALTTSPGETVTLTCRSSTGAVTISNYANWVQEKPDHLF
TGLIGGTNNRPPGVPARFSGSLIGDKAALTITGAQTEDEAIYFCVLWYSNHWVFGGGTKLTV
L.
(SEQ ID NO: 100)
DIVMTQAAFSTPVTLGTSASISCRSSQSLLHSNGITYLYWYLQKPGQSPQLLIYQMS
NLASGVPDRFSSSGSGTDFTLRISRVEAEDVGVYYCAQNLEFPFTFGSGTKLEIKGGGGSGG
GGSGGGGSQVQLQQSGAELMKPGASMKISCKATGYTFSTWIEWVKQRPGHGLEWIGEN
LPGRHITNYNEKFKGKATFTADTSSNTAYMQLSSLTSEDSAVYYCARGRGTYYFDYWGQGT
PLTVSS.
(SEQ ID NO: 101)
QVQLQQSGAELMKPGASMKISCKATGYTFSTYWIEWVKQRPGHGLEWIGENLPG
RHITNYNEKFKGKATFTADTSSNTAYMQLSSLTSEDSAVYYCARGRGTYYFDYWGQGTPLT
VSSGGGGSGGGGSGGGGSDIVMTQAAFSTPVTLGTSASISCRSSQSLLHSNGITYLWYLQ
KPGQSPQLLIYQMSNLASGVPDRFSSSGSGTDFTLRISRVEAEDVGVYYCAQNLEFPFTFGS
GTKLEIK.
(SEQ ID NO: 102)
QIVLTQSPALMSASPGEKVTMTCSATSSVSYIWYQQKPRSSPKPWIYLTSNLASGV
PARFSGSGSGTSYSLTISSMEAEDAATYYCQQWSSNPPTFGGGTKLEIKGGGGSGGGGSGG
GGSEVQLQQSGPELVKPGASMKISCKASGYSFTGYTMNWVKQSHGKNLEWIGLINPYNGG
TNYNQKFKGKATLTVDKSSSTAYMELLSLTSEDSAVYYCAFSYYGSRGFYFDWGQGTTLT
VSS.
(SEQ ID NO: 103)
EVQLQQSGPELVKPGASMKISCKASGYSFTGYTMNWVKQSHGKNLEWIGLINPYN
GGTNYNQKFKGKATLTVDKSSSTAYMELLSLTSEDSAVYYCAFSYYGSRGFYFDWGQGT
TLTVSSGGGGSGGGGSGGGGSQIVLTQSPALMSASPGEKVTMTCSATSSVSYIYWYQQKPR
SSPKPWIYLTSNLASGVPARFSGSGSGTSYSLTISSMEAEDAATYYCQQWSSNPPTFGGGTK
LEIK.
(SEQ ID NO: 104)
DIVMTQSPAMSASPGEKVTMTCSASSSVSYMHWYQQKSGTSPKRWIYDTSKLAS
GVPARFSGSGSGTSYSLTISSMETEDAATYYCQQWSSNPPTFGGGTKLELKGGGGSGGGGS
GGGGSEVQLQQSGPELVKPGASMKISCKASGYSFTGYTMNWVKQSHGKNLEWIGLINPYN
GGTRYNQKFKGKATLTVDKSSSTAYMELLSLTSEDSAVYYCASSSYRNDGNWYFDVWGAG
TTVTVSS.
(SEQ ID NO: 105)
EVQLQQSGPELVKPGASMKISCKASGYSFTGYTMNWVKQSHGKNLEWIGLINPYN
GGTRYNQKFKGKATLTVDKSSSTAYMELLSLTSEDSAVYYCASSSYRNDGNWYFDVWGAG
TTVTVSSGGGGSGGGGSGGGGSDIVMTQSPAIMSASPGEKVTMTCSASSSVSYMHWYQQK
SGTSPKRWIYDTSKLASGVPARFSGSGSGTSYSLTISSMETEDAATYYCQQWSSNPPTFGGG
TKLELK.
(SEQ ID NO: 106)
QIVLSQSPAILSASPGEKVTMTCRATSSVSYMYWYQQKPGSSPKPWIYATSNLASG
VPARFSGSGSGTSYSLTISRVEAEDAATYYCQQWSSNPPTFGGGTKLEKKGGGGSGGGGSG
GGGSEVQLQQSGPELVKPGASMKISCKASGYSITGYTMNWVKQSHGKNLEWIGLVNPYNG
GTSYNQKFKGKATLTVDKSSSTAYMELLSLKSEDSAVYYCAISRYGSESWYFDVWGAGTT
VTVSS.
(SEQ ID NO: 107)
EVQLQQSGPELVKPGASMKISCKASGYSITGYTMNWVKQSHGKNLEWIGLVNPYN
GGTSYNQKFKGKATLTVDKSSSTAYMELLSLKSEDSAVYYCAISRYGSESWYFDVWGAGT
TVTVSSGGGGSGGGGSGGGGSQIVLSQSPAILSASPGEKVTMTCRATSSVSYMYWYQQKPG
SSPKPWIYATSNLASGVPARFSGSGSGTSYSLTISRVEAEDAATYYCQQWSSNPPTFGGGTKL
EKK.
(SEQ ID NO: 108)
DIVLTQSPASLVVSLGQRATISCRTSKSVSSSAYSYMHWYQQKPGQPPKVLIYLASN
LESGVPARFSGSGSGTDFTLNIHPVEEEDAATYYCQHSRELPFTFGSGTKLEIKGGGGSGGG
GSGGGGSQVHLQQSGAELMKPGASVKISCKATGYTFNIYWIDWVKQRPGHGLEWIGEILP
GSGNTHYNENFKGKATMTADTSSNTAYMQLTSLTSEDSAVYYCARTDGRGYFDYWGQGTT
LTVSS.
(SEQ ID NO: 109)
QVHLQQSGAELMKPGASVKISCKATGYTFNIYWIDWVKQRPGHGLEWIGEILPGS
GNTHYNENFKGKATMTADTSSNTAYMQLTSLTSEDSAVYYCARTDGRGYFDYWGQGTTLT
VSSGGGGSGGGGSGGGGSDIVLTQSPASLVVSLGQRATISCRTSKSVSSSAYSYMHWYQQK
PGQPPKVLIYLASNLESGVPARFSGSGSGTDFTLNIHPVEEEDAATYYCQHSRELPFTFGSGT
KLEIK.
(SEQ ID NO: 110)
QIVLTQSPPIMSASPGEKVTMTCSASSNISYMHWYQQKSGTSPKRWIYDTSKLASG
VPARFSGSGSGTSYSLTISSMEAEDAATYYCQQWSSVPLTFGAGTKLEIKGGGGSGGGGSG
GGGSQVQLQQSGAELMKPGASVKISCKATGYTFSSYWIEWVKQRPGHGLEWLGEFLPRSG
KTNYNEEFRGKATFTADTSSNTAYMQLSSLTSEDSAVYYCARTDPPYFGVWGAGTMVAVSS.
(SEQ ID NO: 111)
QVQLQQSGAELMKPGASVKISCKATGYTFSSYWIEWVKQRPGHGLEWLGEFLPRS
GKTNYNEEFRGKATFTADTSSNTAYMQLSSLTSEDSAVYYCARTDPPYFGVWGAGTMVAVS
SGGGGSGGGGSGGGGSQIVLTQSPPIMSASPGEKVTMTCSASSNISYMHWYQQKSGTSPKR
WIYDTSKLASGVPARFSGSGSGTSYSLTISSMEAEDAATYYCQQWSSVPLTFGAGTKLEIK.

Wherein, in the present application, the antibody of the amino acid sequence shown in the above SEQ ID NO: 94 or 95 is referred to as a 2008 single chain antibody fragment, the antibody of the amino acid sequence shown in the above SEQ ID NO: 96 or 97 is referred to as a 23E12 single chain antibody fragment, the antibody of the amino acid sequence shown in the above SEQ ID NO: 98 or 99 is referred to as a 27H3 single chain antibody fragment, the antibody of the amino acid sequence shown in the above SEQ ID NO: 100 or 101 is referred to as a 21E5 single chain antibody fragment, the antibody of the amino acid sequence shown in the above SEQ ID NO: 102 or 103 is referred to as a 2A5 single chain antibody fragment, the antibody of the amino acid sequence shown in the above SEQ ID NO: 104 or 105 is referred to as a 4H4 single chain antibody fragment, the antibody of the amino acid sequence shown in the above SEQ ID NO: 106 or 107 is referred to as a 22D12 single chain antibody fragment, the antibody of the amino acid sequence shown in the above SEQ ID NO: 108 or 109 is referred to as a 1B9 single chain antibody fragment, the antibody of the amino acid sequence shown in the above SEQ ID NO: 110 or 111 is referred to as a 15D4 single chain antibody fragment. Among them, the antibody of the amino acid sequence shown in SEQ ID NO: 94, 96, 98, 100, 102, 104, 106, 108, 110 can be expressed as VL-Link-VH from the N-terminus to the C-terminus(VL represents the light chain variable region, VH represents the heavy chain variable region, and Link represents the linking chain connecting VL and VH), the antibody of the amino acid sequence shown in SEQ ID NO: 95, 97, 99, 101, 103, 105, 107, 109, 111 can be expressed as VH-Link-VL from the N-terminus to the C-terminus(VL represents the light chain variable region, VH represents the heavy chain variable region, and Link represents the linking chain connecting VL and VH).

According to the embodiment of the present invention, the small molecule antibody includes at least one of a Fab antibody, a Fv antibody, a single domain antibody, and a minimum recognition unit.

In a second aspect of the invention, the invention provides a nucleic acid molecule. According to the embodiment of the present invention, the nucleic acid molecule encodes the aforementioned antibody or antigen-binding fragment thereof. The antibody or antigen-binding fragment encoded by the nucleic acid molecule according to the embodiment of the present invention can specifically targeted-bind to the TrkA receptor, block the binding of NGF and TrkA, and has the advantages of low immunogenicity and low ADCC.

According to the embodiment of the present invention, the nucleic acid molecule may further include at least one of the following additional technical features:

according to the embodiment of the present invention, the nucleic acid molecule is DNA.

According to the embodiment of the present invention, the nucleic acid molecule has a nucleotide sequence as shown in any one of SEQ ID NO: 112˜120, or a nucleotide sequence as shown in any one of SEQ ID NO: 121˜129, or a nucleotide sequence as shown in any one of SEQ ID NO: 130˜147.

(SEQ ID NO: 112)
GAAGTGCTGCTGCAGCAGAGCGGACCAGAGCTGGTCAAGCCCGGCGCCTCCAT
GAAGATCAGCTGTAAGGCCTCCGTGTACAGCTTCACAGCCTACACAATGAACTGGGTGA
AGCAGAGCCACGGCAAGAATCTGGAGTGGATCGGACTGATCAACCCACACAATGGCGG
CACTAGGTACAACCAGAAGTTCAAGGGCAAGGCCACACTGACTCTGGATAAGTCCAGC
AGCACTGCCTACATGGATCTGCTGTCTCTGACAAGCGAGGACAGCGCCGTCTACTATTG
CGCCATCTCTAGGTACGGCAGCAGCAGCTTCTACTTCGATGTGTGGGGCGCCGGCACAA
CAGTGGCTGTGAGCAGCGCCAGCACCAAGGGCCCCAGCGTGTTCCCTCTGGCTCCTTGT
AGCCGGTCCACCTCCGAGTCCACAGCTGCTCTGGGCTGCCTCGTGAAGGACTACTTTCC
CGAACCCGTTACCGTGAGCTGGAATAGCGGCGCTTTAACCTCCGGAGTGCACACCTTCC
CCGCTGTGCTCCAGTCCTCCGGTTTATACTCTTTATCCTCCGTGGTGACCGTGCCTTCCTC
CAGCCTCGGCACCAAGACCTACACTTGTAACGTGGACCACAAGCCCAGCAACACCAAG
GTGGACAAGAGGGTGGAGTCCAAGTACGGACCTCCTTGTCCCCCTTGCCCCGCCCCCG
AGGCCGCTGGCGGACCCTCCGTGTTCCTCTTCCCCCCCAAACCCAAGGACACTTTAATG
ATCTCCCGGACCCCCGAAGTGACTTGTGTGGTGGTGGACGTGTCCCAAGAAGACCCCG
AGGTGCAGTTTAACTGGTACGTGGATGGCGTGGAGGTGCACAACGCCAAGACCAAGCC
TAGGGAGGAACAGTTCAACTCCACCTACCGGGTGGTGTCCGTGCTCACCGTGCTGCATC
AAGATTGGCTGAACGGCAAGGAGTACAAGTGCAAGGTGAGCAACAAGGGACTGCCCA
GCTCCATCGAGAAGACCATCAGCAAGGCCAAAGGCCAGCCCCGGGAACCTCAAGTTTA
TACACTGCCCCCCAGCCAAGAAGAGATGACCAAGAACCAAGTTTCTTTAACTTGTTTAG
TGAAGGGCTTCTACCCTAGCGACATCGCTGTGGAGTGGGAGTCCAATGGCCAGCCCGAA
AACAATTATAAGACCACCCCCCCCGTGCTGGACTCCGATGGTTCTTTTTTTTTATACTCCA
AGCTGACAGTGGACAAGTCTCGTTGGCAAGAAGGCAACGTGTTCTCTTGTAGCGTGATG
CACGAGGCTTTACACAACCACTACACCCAGAAGTCTTTATCTCTGTCTTTAGGCTGATGA
GAATTC.
(SEQ ID NO: 113)
CAAGTGCAGCTCCAGCAGAGCGGAGCTGAGCTGGTGAGACCCGGCACTAGCGT
GAAGATCAGCTGTAAGGCCAGCGGCTACGCCTTCACTAATTACTGGCTGGGCTGGATGA
AGCAGAGACCCGGCCATGGACTGGAGTGGATCGGCGACTTCTACCCAAGGACTGGCAA
CACTTTCTACAACGAGAACTTTAAGGGCAAGGTGACTCTGACTGCCGATAAGTCCAGCA
ACACTGCCTACATGCAGCTGTCCTCTCTGACTAGCGAAGATAGCGCCGTGTATCTGTGTG
CTAGGGCTGGCACTGGCTTCGATTACTGGGGCCAAGGCACAACACTGACAGTGAGCAG
CGCCAGCACCAAGGGCCCCAGCGTGTTCCCTCTGGCTCCTTGTAGCCGGTCCACCTCCG
AGTCCACAGCTGCTCTGGGCTGCCTCGTGAAGGACTACTTTCCCGAACCCGTTACCGTG
AGCTGGAATAGCGGCGCTTTAACCTCCGGAGTGCACACCTTCCCCGCTGTGCTCCAGTC
CTCCGGTTTATACTCTTTATCCTCCGTGGTGACCGTGCCTTCCTCCAGCCTCGGCACCAA
GACCTACACTTGTAACGTGGACCACAAGCCCAGCAACACCAAGGTGGACAAGAGGGTG
GAGTCCAAGTACGGACCTCCTTGTCCCCCTTGCCCCGCCCCCGAGGCCGCTGGCGGACC
CTCCGTGTTCCTCTTCCCCCCCAAACCCAAGGACACTTTAATGATCTCCCGGACCCCCGA
AGTGACTTGTGTGGTGGTGGACGTGTCCCAAGAAGACCCCGAGGTGCAGTTTAACTGG
TACGTGGATGGCGTGGAGGTGCACAACGCCAAGACCAAGCCTAGGGAGGAACAGTTCA
ACTCCACCTACCGGGTGGTGTCCGTGCTCACCGTGCTGCATCAAGATTGGCTGAACGGC
AAGGAGTACAAGTGCAAGGTGAGCAACAAGGGACTGCCCAGCTCCATCGAGAAGACC
ATCAGCAAGGCCAAAGGCCAGCCCCGGGAACCTCAAGTTTATACACTGCCCCCCAGCC
AAGAAGAGATGACCAAGAACCAAGTTTCTTTAACTTGTTTAGTGAAGGGCTTCTACCCT
AGCGACATCGCTGTGGAGTGGGAGTCCAATGGCCAGCCCGAAAACAATTATAAGACCA
CCCCCCCCGTGCTGGACTCCGATGGTTCTTTTTTTTTATACTCCAAGCTGACAGTGGACA
AGTCTCGTTGGCAAGAAGGCAACGTGTTCTCTTGTAGCGTGATGCACGAGGCTTTACAC
AACCACTACACCCAGAAGTCTTTATCTCTGTCTTTAGGCTGATGAGAATTC.
(SEQ ID NO: 114)
CAAGTGCAGCTGCAGCAGAGCGGCGCTGAGCTGATGAAGCCCGGCGCTTCCGT
CAAGATCAGCTGCAAGACTACTGGCTATAGGTTCAGCAGCTACTGGATCGAGTGGGTGA
AGCAGAGACCCGGCCACGGACTGGAATGGCTGGGCGAAATCCTCCCCGGAAGGGGCAT
CATCAACTACAATGAGAACTTTAGGGGCAAGGCCACATTCACAGCCGACACAAGCAGC
AACACTGCCTACGTGCAGCTGAGCTCTCTGACTTCCGAGGACAGCGCCGTGTACTTCTG
CGCCAGAACAGACCCTCCTTATTTCGGCGTGTGGGGCGCCGGAACTACTGTGACTGTGT
CCAGCGCCAGCACCAAGGGCCCCAGCGTGTTCCCTCTGGCTCCTTGTAGCCGGTCCACC
TCCGAGTCCACAGCTGCTCTGGGCTGCCTCGTGAAGGACTACTTTCCCGAACCCGTTAC
CGTGAGCTGGAATAGCGGCGCTTTAACCTCCGGAGTGCACACCTTCCCCGCTGTGCTCC
AGTCCTCCGGTTTATACTCTTTATCCTCCGTGGTGACCGTGCCTTCCTCCAGCCTCGGCA
CCAAGACCTACACTTGTAACGTGGACCACAAGCCCAGCAACACCAAGGTGGACAAGAG
GGTGGAGTCCAAGTACGGACCTCCTTGTCCCCCTTGCCCCGCCCCCGAGGCCGCTGGCG
GACCCTCCGTGTTCCTCTTCCCCCCCAAACCCAAGGACACTTTAATGATCTCCCGGACCC
CCGAAGTGACTTGTGTGGTGGTGGACGTGTCCCAAGAAGACCCCGAGGTGCAGTTTAA
CTGGTACGTGGATGGCGTGGAGGTGCACAACGCCAAGACCAAGCCTAGGGAGGAACAG
TTCAACTCCACCTACCGGGTGGTGTCCGTGCTCACCGTGCTGCATCAAGATTGGCTGAA
CGGCAAGGAGTACAAGTGCAAGGTGAGCAACAAGGGACTGCCCAGCTCCATCGAGAA
GACCATCAGCAAGGCCAAAGGCCAGCCCCGGGAACCTCAAGTTTATACACTGCCCCCC
AGCCAAGAAGAGATGACCAAGAACCAAGTTTCTTTAACTTGTTTAGTGAAGGGCTTCTA
CCCTAGCGACATCGCTGTGGAGTGGGAGTCCAATGGCCAGCCCGAAAACAATTATAAGA
CCACCCCCCCCGTGCTGGACTCCGATGGTTCTTTTTTTTTATACTCCAAGCTGACAGTGG
ACAAGTCTCGTTGGCAAGAAGGCAACGTGTTCTCTTGTAGCGTGATGCACGAGGCTTTA
CACAACCACTACACCCAGAAGTCTTTATCTCTGTCTTTAGGCTGATGAGAATTC.
(SEQ ID NO: 115)
CAAGTGCAGCTGCAGCAGAGCGGAGCCGAGCTGATGAAGCCCGGCGCCTCCAT
GAAGATCAGCTGCAAGGCCACTGGCTACACATTCAGCACATACTGGATCGAGTGGGTCA
AGCAGAGACCCGGCCACGGACTGGAGTGGATCGGAGAGAATCTGCCCGGAAGGCACAT
CACTAACTACAACGAGAAGTTCAAGGGCAAGGCCACTTTCACAGCCGACACTAGCAGC
AACACTGCCTACATGCAGCTCAGCTCTCTGACAAGCGAAGATAGCGCCGTGTACTACTG
TGCTAGGGGAAGGGGCACTTACTACTTCGATTACTGGGGCCAAGGCACTCCACTGACTG
TGTCCAGCGCCAGCACCAAGGGCCCCAGCGTGTTCCCTCTGGCTCCTTGTAGCCGGTCC
ACCTCCGAGTCCACAGCTGCTCTGGGCTGCCTCGTGAAGGACTACTTTCCCGAACCCGT
TACCGTGAGCTGGAATAGCGGCGCTTTAACCTCCGGAGTGCACACCTTCCCCGCTGTGC
TCCAGTCCTCCGGTTTATACTCTTTATCCTCCGTGGTGACCGTGCCTTCCTCCAGCCTCGG
CACCAAGACCTACACTTGTAACGTGGACCACAAGCCCAGCAACACCAAGGTGGACAAG
AGGGTGGAGTCCAAGTACGGACCTCCTTGTCCCCCTTGCCCCGCCCCCGAGGCCGCTGG
CGGACCCTCCGTGTTCCTCTTCCCCCCCAAACCCAAGGACACTTTAATGATCTCCCGGAC
CCCCGAAGTGACTTGTGTGGTGGTGGACGTGTCCCAAGAAGACCCCGAGGTGCAGTTT
AACTGGTACGTGGATGGCGTGGAGGTGCACAACGCCAAGACCAAGCCTAGGGAGGAAC
AGTTCAACTCCACCTACCGGGTGGTGTCCGTGCTCACCGTGCTGCATCAAGATTGGCTG
AACGGCAAGGAGTACAAGTGCAAGGTGAGCAACAAGGGACTGCCCAGCTCCATCGAG
AAGACCATCAGCAAGGCCAAAGGCCAGCCCCGGGAACCTCAAGTTTATACACTGCCCC
CCAGCCAAGAAGAGATGACCAAGAACCAAGTTTCTTTAACTTGTTTAGTGAAGGGCTTC
TACCCTAGCGACATCGCTGTGGAGTGGGAGTCCAATGGCCAGCCCGAAAACAATTATAA
GACCACCCCCCCCGTGCTGGACTCCGATGGTTCTTTTTTTTTATACTCCAAGCTGACAGT
GGACAAGTCTCGTTGGCAAGAAGGCAACGTGTTCTCTTGTAGCGTGATGCACGAGGCTT
TACACAACCACTACACCCAGAAGTCTTTATCTCTGTCTTTAGGCTGATGAGAATTC.
(SEQ ID NO: 116)
GAGGTGCAGCTCCAACAGAGCGGACCAGAGCTGGTGAAACCCGGCGCCAGCA
TGAAGATCAGCTGTAAGGCCTCCGGCTACAGCTTCACTGGCTACACTATGAACTGGGTG
AAGCAGTCCCACGGCAAGAATCTGGAGTGGATCGGACTGATCAACCCATACAACGGCG
GCACAAACTACAACCAGAAGTTCAAGGGCAAGGCCACTCTGACAGTCGATAAGAGCTC
CAGCACTGCCTACATGGAGCTGCTGAGCCTCACTAGCGAGGACAGCGCTGTGTACTACT
GTGCCTTCTCCTACTACGGCTCTAGGGGCTTCTACTTCGATTACTGGGGCCAAGGCACAA
CACTGACAGTGTCCAGCGCCAGCACCAAGGGCCCCAGCGTGTTCCCTCTGGCTCCTTGT
AGCCGGTCCACCTCCGAGTCCACAGCTGCTCTGGGCTGCCTCGTGAAGGACTACTTTCC
CGAACCCGTTACCGTGAGCTGGAATAGCGGCGCTTTAACCTCCGGAGTGCACACCTTCC
CCGCTGTGCTCCAGTCCTCCGGTTTATACTCTTTATCCTCCGTGGTGACCGTGCCTTCCTC
CAGCCTCGGCACCAAGACCTACACTTGTAACGTGGACCACAAGCCCAGCAACACCAAG
GTGGACAAGAGGGTGGAGTCCAAGTACGGACCTCCTTGTCCCCCTTGCCCCGCCCCCG
AGGCCGCTGGCGGACCCTCCGTGTTCCTCTTCCCCCCCAAACCCAAGGACACTTTAATG
ATCTCCCGGACCCCCGAAGTGACTTGTGTGGTGGTGGACGTGTCCCAAGAAGACCCCG
AGGTGCAGTTTAACTGGTACGTGGATGGCGTGGAGGTGCACAACGCCAAGACCAAGCC
TAGGGAGGAACAGTTCAACTCCACCTACCGGGTGGTGTCCGTGCTCACCGTGCTGCATC
AAGATTGGCTGAACGGCAAGGAGTACAAGTGCAAGGTGAGCAACAAGGGACTGCCCA
GCTCCATCGAGAAGACCATCAGCAAGGCCAAAGGCCAGCCCCGGGAACCTCAAGTTTA
TACACTGCCCCCCAGCCAAGAAGAGATGACCAAGAACCAAGTTTCTTTAACTTGTTTAG
TGAAGGGCTTCTACCCTAGCGACATCGCTGTGGAGTGGGAGTCCAATGGCCAGCCCGAA
AACAATTATAAGACCACCCCCCCCGTGCTGGACTCCGATGGTTCTTTTTTTTTATACTCCA
AGCTGACAGTGGACAAGTCTCGTTGGCAAGAAGGCAACGTGTTCTCTTGTAGCGTGATG
CACGAGGCTTTACACAACCACTACACCCAGAAGTCTTTATCTCTGTCTTTAGGCTGATGA
GAATTC.
(SEQ ID NO: 117)
GAAGTGCAGCTGCAACAGAGCGGCCCAGAGCTCGTGAAGCCCGGCGCCAGCA
TGAAGATCAGCTGCAAGGCCAGCGGCTACAGCTTCACTGGCTACACAATGAACTGGGTC
AAGCAGAGCCACGGAAAAAATCTGGAGTGGATCGGACTGATCAACCCTTACAACGGCG
GCACAAGGTACAATCAGAAGTTCAAGGGCAAGGCCACTCTCACTGTGGATAAGAGCAG
CAGCACTGCCTACATGGAGCTGCTGTCTCTGACAAGCGAGGATAGCGCCGTGTACTACT
GCGCCAGCTCCTCCTATAGGAACGACGGCAACTGGTACTTCGATGTGTGGGGCGCCGGC
ACTACTGTGACAGTGAGCTCCGCCAGCACCAAGGGCCCCAGCGTGTTCCCTCTGGCTCC
TTGTAGCCGGTCCACCTCCGAGTCCACAGCTGCTCTGGGCTGCCTCGTGAAGGACTACT
TTCCCGAACCCGTTACCGTGAGCTGGAATAGCGGCGCTTTAACCTCCGGAGTGCACACC
TTCCCCGCTGTGCTCCAGTCCTCCGGTTTATACTCTTTATCCTCCGTGGTGACCGTGCCTT
CCTCCAGCCTCGGCACCAAGACCTACACTTGTAACGTGGACCACAAGCCCAGCAACAC
CAAGGTGGACAAGAGGGTGGAGTCCAAGTACGGACCTCCTTGTCCCCCTTGCCCCGCC
CCCGAGGCCGCTGGCGGACCCTCCGTGTTCCTCTTCCCCCCCAAACCCAAGGACACTTT
AATGATCTCCCGGACCCCCGAAGTGACTTGTGTGGTGGTGGACGTGTCCCAAGAAGAC
CCCGAGGTGCAGTTTAACTGGTACGTGGATGGCGTGGAGGTGCACAACGCCAAGACCA
AGCCTAGGGAGGAACAGTTCAACTCCACCTACCGGGTGGTGTCCGTGCTCACCGTGCTG
CATCAAGATTGGCTGAACGGCAAGGAGTACAAGTGCAAGGTGAGCAACAAGGGACTGC
CCAGCTCCATCGAGAAGACCATCAGCAAGGCCAAAGGCCAGCCCCGGGAACCTCAAGT
TTATACACTGCCCCCCAGCCAAGAAGAGATGACCAAGAACCAAGTTTCTTTAACTTGTT
TAGTGAAGGGCTTCTACCCTAGCGACATCGCTGTGGAGTGGGAGTCCAATGGCCAGCCC
GAAAACAATTATAAGACCACCCCCCCCGTGCTGGACTCCGATGGTTCTTTTTTTTTATACT
CCAAGCTGACAGTGGACAAGTCTCGTTGGCAAGAAGGCAACGTGTTCTCTTGTAGCGT
GATGCACGAGGCTTTACACAACCACTACACCCAGAAGTCTTTATCTCTGTCTTTAGGCTG
ATGAGAATTC.
(SEQ ID NO: 118)
GAAGTGCAGCTGCAGCAGAGCGGACCAGAGCTGGTCAAGCCCGGCGCCAGCA
TGAAGATCAGCTGTAAGGCCAGCGGCTACAGCATCACTGGCTACACTATGAACTGGGTG
AAGCAGAGCCACGGCAAGAACCTCGAGTGGATTGGCCTCGTGAACCCATACAACGGCG
GCACTTCCTACAACCAGAAGTTCAAAGGCAAGGCCACACTCACAGTCGATAAGTCCAG
CTCCACAGCCTACATGGAGCTGCTGTCTCTGAAGAGCGAGGATAGCGCTGTCTACTACT
GTGCCATCAGCAGATACGGCAGCGAGAGCTGGTACTTCGACGTGTGGGGCGCCGGCAC
AACAGTGACAGTGAGCAGCGCCAGCACAAAGGGCCCCAGCGTGTTCCCTCTGGCTCCT
TGTAGCCGGTCCACCTCCGAGTCCACAGCTGCTCTGGGCTGCCTCGTGAAGGACTACTT
TCCCGAACCCGTTACCGTGAGCTGGAATAGCGGCGCTTTAACCTCCGGAGTGCACACCT
TCCCCGCTGTGCTCCAGTCCTCCGGTTTATACTCTTTATCCTCCGTGGTGACCGTGCCTTC
CTCCAGCCTCGGCACCAAGACCTACACTTGTAACGTGGACCACAAGCCCAGCAACACC
AAGGTGGACAAGAGGGTGGAGTCCAAGTACGGACCTCCTTGTCCCCCTTGCCCCGCCC
CCGAGGCCGCTGGCGGACCCTCCGTGTTCCTCTTCCCCCCCAAACCCAAGGACACTTTA
ATGATCTCCCGGACCCCCGAAGTGACTTGTGTGGTGGTGGACGTGTCCCAAGAAGACCC
CGAGGTGCAGTTTAACTGGTACGTGGATGGCGTGGAGGTGCACAACGCCAAGACCAAG
CCTAGGGAGGAACAGTTCAACTCCACCTACCGGGTGGTGTCCGTGCTCACCGTGCTGCA
TCAAGATTGGCTGAACGGCAAGGAGTACAAGTGCAAGGTGAGCAACAAGGGACTGCCC
AGCTCCATCGAGAAGACCATCAGCAAGGCCAAAGGCCAGCCCCGGGAACCTCAAGTTT
ATACACTGCCCCCCAGCCAAGAAGAGATGACCAAGAACCAAGTTTCTTTAACTTGTTTA
GTGAAGGGCTTCTACCCTAGCGACATCGCTGTGGAGTGGGAGTCCAATGGCCAGCCCGA
AAACAATTATAAGACCACCCCCCCCGTGCTGGACTCCGATGGTTCTTTTTTTTTATACTCC
AAGCTGACAGTGGACAAGTCTCGTTGGCAAGAAGGCAACGTGTTCTCTTGTAGCGTGAT
GCACGAGGCTTTACACAACCACTACACCCAGAAGTCTTTATCTCTGTCTTTAGGCTGATG
AGAATTC.
(SEQ ID NO: 119)
CAAGTGCATCTGCAGCAGAGCGGCGCTGAGCTGATGAAGCCCGGCGCCAGCGT
GAAGATTAGCTGCAAGGCCACTGGCTACACATTCAACATCTACTGGATCGACTGGGTGA
AGCAGAGGCCCGGCCACGGACTGGAATGGATCGGCGAAATTCTGCCCGGCAGCGGCAA
CACTCACTACAACGAGAACTTCAAGGGCAAGGCCACAATGACAGCCGACACAAGCTCC
AACACTGCTTACATGCAGCTGACTTCTCTGACTAGCGAGGACAGCGCCGTGTACTATTG
CGCTAGGACAGACGGAAGGGGCTACTTCGATTACTGGGGCCAAGGCACTACACTCACA
GTGAGCAGCGCCAGCACTAAGGGCCCCAGCGTGTTCCCTCTGGCTCCTTGTAGCCGGTC
CACCTCCGAGTCCACAGCTGCTCTGGGCTGCCTCGTGAAGGACTACTTTCCCGAACCCG
TTACCGTGAGCTGGAATAGCGGCGCTTTAACCTCCGGAGTGCACACCTTCCCCGCTGTG
CTCCAGTCCTCCGGTTTATACTCTTTATCCTCCGTGGTGACCGTGCCTTCCTCCAGCCTCG
GCACCAAGACCTACACTTGTAACGTGGACCACAAGCCCAGCAACACCAAGGTGGACAA
GAGGGTGGAGTCCAAGTACGGACCTCCTTGTCCCCCTTGCCCCGCCCCCGAGGCCGCTG
GCGGACCCTCCGTGTTCCTCTTCCCCCCCAAACCCAAGGACACTTTAATGATCTCCCGG
ACCCCCGAAGTGACTTGTGTGGTGGTGGACGTGTCCCAAGAAGACCCCGAGGTGCAGT
TTAACTGGTACGTGGATGGCGTGGAGGTGCACAACGCCAAGACCAAGCCTAGGGAGGA
ACAGTTCAACTCCACCTACCGGGTGGTGTCCGTGCTCACCGTGCTGCATCAAGATTGGC
TGAACGGCAAGGAGTACAAGTGCAAGGTGAGCAACAAGGGACTGCCCAGCTCCATCGA
GAAGACCATCAGCAAGGCCAAAGGCCAGCCCCGGGAACCTCAAGTTTATACACTGCCC
CCCAGCCAAGAAGAGATGACCAAGAACCAAGTTTCTTTAACTTGTTTAGTGAAGGGCTT
CTACCCTAGCGACATCGCTGTGGAGTGGGAGTCCAATGGCCAGCCCGAAAACAATTATA
AGACCACCCCCCCCGTGCTGGACTCCGATGGTTCTTTTTTTTTATACTCCAAGCTGACAG
TGGACAAGTCTCGTTGGCAAGAAGGCAACGTGTTCTCTTGTAGCGTGATGCACGAGGCT
TTACACAACCACTACACCCAGAAGTCTTTATCTCTGTCTTTAGGCTGATGAGAATTC.
(SEQ ID NO: 120)
CAGGTGCAGCTGCAGCAGTCCGGCGCTGAGCTCATGAAGCCCGGCGCCAGCGT
GAAGATCAGCTGCAAGGCCACTGGCTACACATTCAGCAGCTACTGGATCGAGTGGGTGA
AGCAGAGACCCGGCCACGGACTGGAATGGCTGGGAGAGTTTCTGCCTAGAAGCGGCAA
GACAAACTACAACGAGGAGTTTAGGGGCAAGGCTACATTCACTGCCGACACATCCAGC
AACACTGCCTACATGCAGCTGAGCAGCCTCACAAGCGAGGATTCCGCCGTCTACTACTG
TGCTAGGACTGATCCTCCTTACTTCGGAGTGTGGGGCGCTGGCACAATGGTGGCTGTGA
GCAGCGCCTCCACTAAGGGCCCCAGCGTGTTCCCTCTGGCTCCTTGTAGCCGGTCCACC
TCCGAGTCCACAGCTGCTCTGGGCTGCCTCGTGAAGGACTACTTTCCCGAACCCGTTAC
CGTGAGCTGGAATAGCGGCGCTTTAACCTCCGGAGTGCACACCTTCCCCGCTGTGCTCC
AGTCCTCCGGTTTATACTCTTTATCCTCCGTGGTGACCGTGCCTTCCTCCAGCCTCGGCA
CCAAGACCTACACTTGTAACGTGGACCACAAGCCCAGCAACACCAAGGTGGACAAGAG
GGTGGAGTCCAAGTACGGACCTCCTTGTCCCCCTTGCCCCGCCCCCGAGGCCGCTGGCG
GACCCTCCGTGTTCCTCTTCCCCCCCAAACCCAAGGACACTTTAATGATCTCCCGGACCC
CCGAAGTGACTTGTGTGGTGGTGGACGTGTCCCAAGAAGACCCCGAGGTGCAGTTTAA
CTGGTACGTGGATGGCGTGGAGGTGCACAACGCCAAGACCAAGCCTAGGGAGGAACAG
TTCAACTCCACCTACCGGGTGGTGTCCGTGCTCACCGTGCTGCATCAAGATTGGCTGAA
CGGCAAGGAGTACAAGTGCAAGGTGAGCAACAAGGGACTGCCCAGCTCCATCGAGAA
GACCATCAGCAAGGCCAAAGGCCAGCCCCGGGAACCTCAAGTTTATACACTGCCCCCC
AGCCAAGAAGAGATGACCAAGAACCAAGTTTCTTTAACTTGTTTAGTGAAGGGCTTCTA
CCCTAGCGACATCGCTGTGGAGTGGGAGTCCAATGGCCAGCCCGAAAACAATTATAAGA
CCACCCCCCCCGTGCTGGACTCCGATGGTTCTTTTTTTTTATACTCCAAGCTGACAGTGG
ACAAGTCTCGTTGGCAAGAAGGCAACGTGTTCTCTTGTAGCGTGATGCACGAGGCTTTA
CACAACCACTACACCCAGAAGTCTTTATCTCTGTCTTTAGGCTGATGAGAATTC.
(SEQ ID NO: 121)
CAGATCGTGCTGAGCCAGAGCCCAGCTATTCTGTCCGCCAGCCCCGGCGAGAA
GGTGACTATGACTTGTAGGGCCAGCTCCAGCATCAGCTACATGCACTGGTACCAGCAGA
AGCCCGGCTCCTCCCCAAAGCCTTGGATCAGCGCCACTAGCAATCTGGCCAGCGGCGTG
CCAGCCAGATTCAGCGGAAGCGGCAGCGGCACTAGCTACTCTCTGACTATCTCCGGCGT
GGAAGCTGAGGATGCCGCCACTTACTACTGCCAGCAGTGGTCCAGCAACCCTCCTACTT
TCGGCGGCGGCACAAATCTGGAGATCAAGCGGACCGTGGCTGCCCCCTCCGTGTTCATC
TTCCCCCCTTCCGACGAGCAGCTGAAGTCCGGCACCGCTAGCGTGGTGTGTTTACTGAA
CAACTTCTACCCTCGTGAGGCCAAGGTGCAGTGGAAGGTGGACAACGCTTTACAGTCC
GGCAACTCCCAAGAATCCGTGACCGAGCAAGATTCCAAGGACTCCACCTACTCTTTATC
CTCCACTTTAACTTTATCCAAGGCCGACTACGAGAAGCACAAGGTGTACGCTTGTGAGG
TGACCCATCAAGGTTTATCCTCCCCCGTGACCAAGTCCTTCAATCGTGGCGAGTGCTGAT
GAGAATTC.
(SEQ ID NO: 122)
AGCATCGTCATGACACAAAGCCCTAAGAGCATGAGCATGAGCGTGGGCGAGAG
AGTGACTCTGAGCTGTAAGGCCAGCGAGAATGTGGGCGGCTACGTGAGCTGGTATCAGC
AGAAGCCAGATCAGAGCCCAAAGCTGCTGATCTACGGCGCCAGCAGCAGACACACTGG
CGTGCCAGATAGGTTCACTGGCAGCGGCTCCGAGACAGACTTCACTCTGACTATCAGCA
GCGTCCAAGCCGAAGATCTGGCCGCCTATCACTGCGGCCAGAACTACATCTACCCATTC
ACATTCGGCGGCGGCACAAAGCTGGAGATCAAGCGGACCGTGGCTGCCCCCTCCGTGT
TCATCTTCCCCCCTTCCGACGAGCAGCTGAAGTCCGGCACCGCTAGCGTGGTGTGTTTA
CTGAACAACTTCTACCCTCGTGAGGCCAAGGTGCAGTGGAAGGTGGACAACGCTTTAC
AGTCCGGCAACTCCCAAGAATCCGTGACCGAGCAAGATTCCAAGGACTCCACCTACTCT
TTATCCTCCACTTTAACTTTATCCAAGGCCGACTACGAGAAGCACAAGGTGTACGCTTGT
GAGGTGACCCATCAAGGTTTATCCTCCCCCGTGACCAAGTCCTTCAATCGTGGCGAGTG
CTGATGAGAATTC.
(SEQ ID NO: 123)
CAAGCCGTCGTGACACAAGAGTCCGCTCTGACAACTTCCCCCGGCGAGACTGT
GACACTGACTTGTAGGAGCAGCACTGGCGCCGTGACTATCAGCAACTACGCCAACTGG
GTCCAAGAGAAGCCAGATCATCTGTTCACTGGACTGATCGGCGGCACAAATAATAGGCC
TCCCGGCGTGCCAGCCAGATTTAGCGGCTCTCTGATTGGCGATAAGGCTGCTCTGACAAT
CACTGGCGCCCAGACTGAGGACGAGGCCATCTACTTCTGCGTCCTCTGGTACAGCAACC
ACTGGGTGTTCGGCGGCGGCACTAAGCTGACTGTGCTGCGGACCGTGGCTGCCCCCTCC
GTGTTCATCTTCCCCCCTTCCGACGAGCAGCTGAAGTCCGGCACCGCTAGCGTGGTGTG
TTTACTGAACAACTTCTACCCTCGTGAGGCCAAGGTGCAGTGGAAGGTGGACAACGCTT
TACAGTCCGGCAACTCCCAAGAATCCGTGACCGAGCAAGATTCCAAGGACTCCACCTAC
TCTTTATCCTCCACTTTAACTTTATCCAAGGCCGACTACGAGAAGCACAAGGTGTACGCT
TGTGAGGTGACCCATCAAGGTTTATCCTCCCCCGTGACCAAGTCCTTCAATCGTGGCGA
GTGCTGATGAGAATTC.
(SEQ ID NO: 124)
GATATCGTCATGACTCAAGCCGCCTTCAGCACTCCAGTCACTCTCGGCACAAGC
GCCAGCATCAGCTGTAGGTCCAGCCAGTCTCTGCTGCACAGCAACGGCATCACTTATCT
GTACTGGTATCTGCAGAAGCCCGGCCAAAGCCCACAGCTGCTGATCTACCAGATGAGCA
ATCTGGCCAGCGGCGTGCCAGATAGATTCAGCAGCAGCGGAAGCGGAACAGACTTCAC
ACTGAGGATCTCTAGGGTGGAAGCCGAAGATGTGGGCGTCTATTACTGCGCCCAGAACC
TCGAGTTCCCTTTCACATTCGGCAGCGGCACTAAGCTGGAGATCAAGCGGACCGTGGCT
GCCCCCTCCGTGTTCATCTTCCCCCCTTCCGACGAGCAGCTGAAGTCCGGCACCGCTAG
CGTGGTGTGTTTACTGAACAACTTCTACCCTCGTGAGGCCAAGGTGCAGTGGAAGGTGG
ACAACGCTTTACAGTCCGGCAACTCCCAAGAATCCGTGACCGAGCAAGATTCCAAGGA
CTCCACCTACTCTTTATCCTCCACTTTAACTTTATCCAAGGCCGACTACGAGAAGCACAA
GGTGTACGCTTGTGAGGTGACCCATCAAGGTTTATCCTCCCCCGTGACCAAGTCCTTCA
ATCGTGGCGAGTGCTGATGAGAATTC.
(SEQ ID NO: 125)
CAGATCGTGCTGACACAGAGCCCAGCTCTGATGAGCGCCAGCCCCGGCGAGAA
GGTCACAATGACTTGCAGCGCCACATCCAGCGTGAGCTACATCTACTGGTACCAGCAGA
AGCCTAGGAGCAGCCCTAAGCCTTGGATCTACCTCACAAGCAATCTGGCCAGCGGAGTG
CCAGCTAGGTTCAGCGGAAGCGGCAGCGGCACAAGCTACTCTCTGACAATCTCCAGCAT
GGAAGCCGAAGATGCCGCCACTTACTACTGCCAGCAGTGGAGCAGCAATCCACCTACAT
TCGGAGGCGGCACTAAGCTGGAGATCAAGCGGACCGTGGCTGCCCCCTCCGTGTTCATC
TTCCCCCCTTCCGACGAGCAGCTGAAGTCCGGCACCGCTAGCGTGGTGTGTTTACTGAA
CAACTTCTACCCTCGTGAGGCCAAGGTGCAGTGGAAGGTGGACAACGCTTTACAGTCC
GGCAACTCCCAAGAATCCGTGACCGAGCAAGATTCCAAGGACTCCACCTACTCTTTATC
CTCCACTTTAACTTTATCCAAGGCCGACTACGAGAAGCACAAGGTGTACGCTTGTGAGG
TGACCCATCAAGGTTTATCCTCCCCCGTGACCAAGTCCTTCAATCGTGGCGAGTGCTGAT
GAGAATTC.
(SEQ ID NO: 126)
GATATCGTGATGACTCAGTCCCCAGCCATCATGTCCGCCAGCCCCGGCGAGAAG
GTGACTATGACTTGCTCCGCCAGCAGCAGCGTGAGCTACATGCACTGGTACCAGCAGAA
GAGCGGCACATCCCCAAAGAGGTGGATCTACGACACAAGCAAGCTGGCCAGCGGCGTG
CCAGCCAGATTCAGCGGCTCCGGCAGCGGAACAAGCTACTCTCTGACTATCAGCAGCAT
GGAGACAGAGGACGCTGCCACTTACTACTGCCAGCAGTGGAGCAGCAATCCTCCAACT
TTCGGCGGAGGCACTAAGCTGGAGCTGAAGCGGACCGTGGCTGCCCCCTCCGTGTTCAT
CTTCCCCCCTTCCGACGAGCAGCTGAAGTCCGGCACCGCTAGCGTGGTGTGTTTACTGA
ACAACTTCTACCCTCGTGAGGCCAAGGTGCAGTGGAAGGTGGACAACGCTTTACAGTC
CGGCAACTCCCAAGAATCCGTGACCGAGCAAGATTCCAAGGACTCCACCTACTCTTTAT
CCTCCACTTTAACTTTATCCAAGGCCGACTACGAGAAGCACAAGGTGTACGCTTGTGAG
GTGACCCATCAAGGTTTATCCTCCCCCGTGACCAAGTCCTTCAATCGTGGCGAGTGCTG
ATGAGAATTC.
(SEQ ID NO: 127)
CAGATTGTGCTGTCCCAGTCCCCAGCCATTCTGAGCGCCAGCCCCGGCGAGAA
GGTGACTATGACTTGTAGGGCCACAAGCAGCGTGAGCTACATGTACTGGTACCAGCAGA
AGCCCGGCAGCAGCCCTAAGCCTTGGATCTACGCCACAAGCAATCTGGCCAGCGGCGTC
CCAGCTAGATTTAGCGGCAGCGGATCCGGCACTAGCTATTCTCTGACTATCTCTAGGGTC
GAGGCCGAAGATGCCGCCACATACTACTGCCAGCAGTGGTCCTCCAATCCTCCAACATT
CGGCGGCGGAACTAAGCTGGAGAAGAAGAGGACAGTGGCTGCCCCTTCCGTGTTCATC
TTCCCTCCAAGCGACGAGCAGCTGAAGTCCGGCACTGCTAGCGTGGTGTGTTTACTGAA
CAACTTCTACCCTCGTGAGGCCAAGGTGCAGTGGAAGGTGGACAACGCTTTACAGTCC
GGCAACTCCCAAGAATCCGTGACCGAGCAAGATTCCAAGGACTCCACCTACTCTTTATC
CTCCACTTTAACTTTATCCAAGGCCGACTACGAGAAGCACAAGGTGTACGCTTGTGAGG
TGACCCATCAAGGTTTATCCTCCCCCGTGACCAAGTCCTTCAATCGTGGCGAGTGCTGAT
GAGAATTC.
(SEQ ID NO: 128)
GATATCGTGCTGACTCAGAGCCCAGCCTCTCTGGTGGTGTCTCTGGGACAGAGG
GCCACAATCAGCTGTAGGACTTCCAAGAGCGTGAGCAGCTCCGCCTACAGCTACATGCA
CTGGTACCAGCAGAAGCCCGGCCAGCCTCCTAAGGTGCTGATCTATCTGGCCAGCAATC
TGGAGAGCGGCGTCCCAGCTAGATTCAGCGGCTCCGGAAGCGGCACTGACTTCACTCT
GAACATCCACCCAGTGGAAGAGGAGGATGCCGCCACATACTACTGCCAGCACTCTAGGG
AGCTGCCTTTCACATTTGGCAGCGGAACTAAGCTGGAGATCAAGAGGACTGTCGCCGCC
CCTAGCGTGTTCATCTTCCCTCCAAGCGATGAGCAGCTGAAGAGCGGCACTGCTAGCGT
GGTGTGTTTACTGAACAACTTCTACCCTCGTGAGGCCAAGGTGCAGTGGAAGGTGGAC
AACGCTTTACAGTCCGGCAACTCCCAAGAATCCGTGACCGAGCAAGATTCCAAGGACTC
CACCTACTCTTTATCCTCCACTTTAACTTTATCCAAGGCCGACTACGAGAAGCACAAGGT
GTACGCTTGTGAGGTGACCCATCAAGGTTTATCCTCCCCCGTGACCAAGTCCTTCAATCG
TGGCGAGTGCTGATGAGAATTC.
(SEQ ID NO: 129)
CAAATTGTTCTCACACAGAGCCCTCCTATCATGAGCGCCAGCCCCGGCGAGAAG
GTGACTATGACTTGTTCCGCCAGCAGCAACATCAGCTACATGCACTGGTACCAGCAGAA
GTCCGGCACAAGCCCAAAGAGGTGGATCTACGACACAAGCAAGCTGGCCAGCGGCGTG
CCAGCCAGATTTAGCGGCTCCGGAAGCGGCACTAGCTACTCTCTGACAATCAGCAGCAT
GGAAGCCGAGGACGCCGCTACATACTACTGCCAGCAGTGGAGCTCCGTCCCACTGACTT
TCGGCGCTGGCACTAAGCTGGAGATCAAGAGGACTGTGGCCGCCCCTTCCGTGTTCATC
TTCCCTCCTAGCGACGAACAGCTCAAGAGCGGCACTGCTAGCGTGGTGTGTTTACTGAA
CAACTTCTACCCTCGTGAGGCCAAGGTGCAGTGGAAGGTGGACAACGCTTTACAGTCC
GGCAACTCCCAAGAATCCGTGACCGAGCAAGATTCCAAGGACTCCACCTACTCTTTATC
CTCCACTTTAACTTTATCCAAGGCCGACTACGAGAAGCACAAGGTGTACGCTTGTGAGG
TGACCCATCAAGGTTTATCCTCCCCCGTGACCAAGTCCTTCAATCGTGGCGAGTGCTGAT
GAGAATTC.
(SEQ ID NO: 130)
CAGATCGTGCTGAGCCAGAGCCCAGCTATTCTGTCCGCCAGCCCCGGCGAGAA
GGTGACTATGACTTGTAGGGCCAGCTCCAGCATCAGCTACATGCACTGGTACCAGCAGA
AGCCCGGCTCCTCCCCAAAGCCTTGGATCAGCGCCACTAGCAATCTGGCCAGCGGCGTG
CCAGCCAGATTCAGCGGAAGCGGCAGCGGCACTAGCTACTCTCTGACTATCTCCGGCGT
GGAAGCTGAGGATGCCGCCACTTACTACTGCCAGCAGTGGTCCAGCAACCCTCCTACTT
TCGGCGGCGGCACAAATCTGGAGATCAAGGGTGGCGGTGGCTCGGGCGGTGGTGGGTC
GGGTGGCGGCGGATCTGAAGTGCTGCTGCAGCAGAGCGGACCAGAGCTGGTCAAGCCC
GGCGCCTCCATGAAGATCAGCTGTAAGGCCTCCGTGTACAGCTTCACAGCCTACACAAT
GAACTGGGTGAAGCAGAGCCACGGCAAGAATCTGGAGTGGATCGGACTGATCAACCCA
CACAATGGCGGCACTAGGTACAACCAGAAGTTCAAGGGCAAGGCCACACTGACTCTGG
ATAAGTCCAGCAGCACTGCCTACATGGATCTGCTGTCTCTGACAAGCGAGGACAGCGCC
GTCTACTATTGCGCCATCTCTAGGTACGGCAGCAGCAGCTTCTACTTCGATGTGTGGGGC
GCCGGCACAACAGTGGCTGTGAGCAGC.
(SEQ ID NO: 131)
GAAGTGCTGCTGCAGCAGAGCGGACCAGAGCTGGTCAAGCCCGGCGCCTCCAT
GAAGATCAGCTGTAAGGCCTCCGTGTACAGCTTCACAGCCTACACAATGAACTGGGTGA
AGCAGAGCCACGGCAAGAATCTGGAGTGGATCGGACTGATCAACCCACACAATGGCGG
CACTAGGTACAACCAGAAGTTCAAGGGCAAGGCCACACTGACTCTGGATAAGTCCAGC
AGCACTGCCTACATGGATCTGCTGTCTCTGACAAGCGAGGACAGCGCCGTCTACTATTG
CGCCATCTCTAGGTACGGCAGCAGCAGCTTCTACTTCGATGTGTGGGGCGCCGGCACAA
CAGTGGCTGTGAGCAGCGGTGGCGGTGGCTCGGGCGGTGGTGGGTCGGGTGGCGGCG
GATCTCAGATCGTGCTGAGCCAGAGCCCAGCTATTCTGTCCGCCAGCCCCGGCGAGAAG
GTGACTATGACTTGTAGGGCCAGCTCCAGCATCAGCTACATGCACTGGTACCAGCAGAA
GCCCGGCTCCTCCCCAAAGCCTTGGATCAGCGCCACTAGCAATCTGGCCAGCGGCGTGC
CAGCCAGATTCAGCGGAAGCGGCAGCGGCACTAGCTACTCTCTGACTATCTCCGGCGTG
GAAGCTGAGGATGCCGCCACTTACTACTGCCAGCAGTGGTCCAGCAACCCTCCTACTTT
CGGCGGCGGCACAAATCTGGAGATCAAG.
(SEQ ID NO: 132)
AGCATCGTCATGACACAAAGCCCTAAGAGCATGAGCATGAGCGTGGGCGAGAG
AGTGACTCTGAGCTGTAAGGCCAGCGAGAATGTGGGCGGCTACGTGAGCTGGTATCAGC
AGAAGCCAGATCAGAGCCCAAAGCTGCTGATCTACGGCGCCAGCAGCAGACACACTGG
CGTGCCAGATAGGTTCACTGGCAGCGGCTCCGAGACAGACTTCACTCTGACTATCAGCA
GCGTCCAAGCCGAAGATCTGGCCGCCTATCACTGCGGCCAGAACTACATCTACCCATTC
ACATTCGGCGGCGGCACAAAGCTGGAGATCAAGGGTGGCGGTGGCTCGGGCGGTGGTG
GGTCGGGTGGCGGCGGATCTCAAGTGCAGCTCCAGCAGAGCGGAGCTGAGCTGGTGAG
ACCCGGCACTAGCGTGAAGATCAGCTGTAAGGCCAGCGGCTACGCCTTCACTAATTACT
GGCTGGGCTGGATGAAGCAGAGACCCGGCCATGGACTGGAGTGGATCGGCGACTTCTA
CCCAAGGACTGGCAACACTTTCTACAACGAGAACTTTAAGGGCAAGGTGACTCTGACT
GCCGATAAGTCCAGCAACACTGCCTACATGCAGCTGTCCTCTCTGACTAGCGAAGATAG
CGCCGTGTATCTGTGTGCTAGGGCTGGCACTGGCTTCGATTACTGGGGCCAAGGCACAA
CACTGACAGTGAGCAGC.
(SEQ ID NO: 133)
CAAGTGCAGCTCCAGCAGAGCGGAGCTGAGCTGGTGAGACCCGGCACTAGCGT
GAAGATCAGCTGTAAGGCCAGCGGCTACGCCTTCACTAATTACTGGCTGGGCTGGATGA
AGCAGAGACCCGGCCATGGACTGGAGTGGATCGGCGACTTCTACCCAAGGACTGGCAA
CACTTTCTACAACGAGAACTTTAAGGGCAAGGTGACTCTGACTGCCGATAAGTCCAGCA
ACACTGCCTACATGCAGCTGTCCTCTCTGACTAGCGAAGATAGCGCCGTGTATCTGTGTG
CTAGGGCTGGCACTGGCTTCGATTACTGGGGCCAAGGCACAACACTGACAGTGAGCAG
CGGTGGCGGTGGCTCGGGCGGTGGTGGGTCGGGTGGCGGCGGATCTAGCATCGTCATG
ACACAAAGCCCTAAGAGCATGAGCATGAGCGTGGGCGAGAGAGTGACTCTGAGCTGTA
AGGCCAGCGAGAATGTGGGCGGCTACGTGAGCTGGTATCAGCAGAAGCCAGATCAGAG
CCCAAAGCTGCTGATCTACGGCGCCAGCAGCAGACACACTGGCGTGCCAGATAGGTTC
ACTGGCAGCGGCTCCGAGACAGACTTCACTCTGACTATCAGCAGCGTCCAAGCCGAAG
ATCTGGCCGCCTATCACTGCGGCCAGAACTACATCTACCCATTCACATTCGGCGGCGGCA
CAAAGCTGGAGATCAAG.
(SEQ ID NO: 134)
CAAGCCGTCGTGACACAAGAGTCCGCTCTGACAACTTCCCCCGGCGAGACTGT
GACACTGACTTGTAGGAGCAGCACTGGCGCCGTGACTATCAGCAACTACGCCAACTGG
GTCCAAGAGAAGCCAGATCATCTGTTCACTGGACTGATCGGCGGCACAAATAATAGGCC
TCCCGGCGTGCCAGCCAGATTTAGCGGCTCTCTGATTGGCGATAAGGCTGCTCTGACAAT
CACTGGCGCCCAGACTGAGGACGAGGCCATCTACTTCTGCGTCCTCTGGTACAGCAACC
ACTGGGTGTTCGGCGGCGGCACTAAGCTGACTGTGCTGGGTGGCGGTGGCTCGGGCGG
TGGTGGGTCGGGTGGCGGCGGATCTCAAGTGCAGCTGCAGCAGAGCGGCGCTGAGCTG
ATGAAGCCCGGCGCTTCCGTCAAGATCAGCTGCAAGACTACTGGCTATAGGTTCAGCAG
CTACTGGATCGAGTGGGTGAAGCAGAGACCCGGCCACGGACTGGAATGGCTGGGCGAA
ATCCTCCCCGGAAGGGGCATCATCAACTACAATGAGAACTTTAGGGGCAAGGCCACATT
CACAGCCGACACAAGCAGCAACACTGCCTACGTGCAGCTGAGCTCTCTGACTTCCGAG
GACAGCGCCGTGTACTTCTGCGCCAGAACAGACCCTCCTTATTTCGGCGTGTGGGGCGC
CGGAACTACTGTGACTGTGTCCAGC.
(SEQ ID NO: 135)
CAAGTGCAGCTGCAGCAGAGCGGCGCTGAGCTGATGAAGCCCGGCGCTTCCGT
CAAGATCAGCTGCAAGACTACTGGCTATAGGTTCAGCAGCTACTGGATCGAGTGGGTGA
AGCAGAGACCCGGCCACGGACTGGAATGGCTGGGCGAAATCCTCCCCGGAAGGGGCAT
CATCAACTACAATGAGAACTTTAGGGGCAAGGCCACATTCACAGCCGACACAAGCAGC
AACACTGCCTACGTGCAGCTGAGCTCTCTGACTTCCGAGGACAGCGCCGTGTACTTCTG
CGCCAGAACAGACCCTCCTTATTTCGGCGTGTGGGGCGCCGGAACTACTGTGACTGTGT
CCAGCGGTGGCGGTGGCTCGGGCGGTGGTGGGTCGGGTGGCGGCGGATCTCAAGCCGT
CGTGACACAAGAGTCCGCTCTGACAACTTCCCCCGGCGAGACTGTGACACTGACTTGTA
GGAGCAGCACTGGCGCCGTGACTATCAGCAACTACGCCAACTGGGTCCAAGAGAAGCC
AGATCATCTGTTCACTGGACTGATCGGCGGCACAAATAATAGGCCTCCCGGCGTGCCAG
CCAGATTTAGCGGCTCTCTGATTGGCGATAAGGCTGCTCTGACAATCACTGGCGCCCAG
ACTGAGGACGAGGCCATCTACTTCTGCGTCCTCTGGTACAGCAACCACTGGGTGTTCGG
CGGCGGCACTAAGCTGACTGTGCTG.
(SEQ ID NO: 136)
GATATCGTCATGACTCAAGCCGCCTTCAGCACTCCAGTCACTCTCGGCACAAGC
GCCAGCATCAGCTGTAGGTCCAGCCAGTCTCTGCTGCACAGCAACGGCATCACTTATCT
GTACTGGTATCTGCAGAAGCCCGGCCAAAGCCCACAGCTGCTGATCTACCAGATGAGCA
ATCTGGCCAGCGGCGTGCCAGATAGATTCAGCAGCAGCGGAAGCGGAACAGACTTCAC
ACTGAGGATCTCTAGGGTGGAAGCCGAAGATGTGGGCGTCTATTACTGCGCCCAGAACC
TCGAGTTCCCTTTCACATTCGGCAGCGGCACTAAGCTGGAGATCAAGGGTGGCGGTGGC
TCGGGCGGTGGTGGGTCGGGTGGCGGCGGATCTCAAGTGCAGCTGCAGCAGAGCGGAG
CCGAGCTGATGAAGCCCGGCGCCTCCATGAAGATCAGCTGCAAGGCCACTGGCTACAC
ATTCAGCACATACTGGATCGAGTGGGTCAAGCAGAGACCCGGCCACGGACTGGAGTGG
ATCGGAGAGAATCTGCCCGGAAGGCACATCACTAACTACAACGAGAAGTTCAAGGGCA
AGGCCACTTTCACAGCCGACACTAGCAGCAACACTGCCTACATGCAGCTCAGCTCTCTG
ACAAGCGAAGATAGCGCCGTGTACTACTGTGCTAGGGGAAGGGGCACTTACTACTTCGA
TTACTGGGGCCAAGGCACTCCACTGACTGTGTCCAGC.
(SEQ ID NO: 137)
CAAGTGCAGCTGCAGCAGAGCGGAGCCGAGCTGATGAAGCCCGGCGCCTCCAT
GAAGATCAGCTGCAAGGCCACTGGCTACACATTCAGCACATACTGGATCGAGTGGGTCA
AGCAGAGACCCGGCCACGGACTGGAGTGGATCGGAGAGAATCTGCCCGGAAGGCACAT
CACTAACTACAACGAGAAGTTCAAGGGCAAGGCCACTTTCACAGCCGACACTAGCAGC
AACACTGCCTACATGCAGCTCAGCTCTCTGACAAGCGAAGATAGCGCCGTGTACTACTG
TGCTAGGGGAAGGGGCACTTACTACTTCGATTACTGGGGCCAAGGCACTCCACTGACTG
TGTCCAGCGGTGGCGGTGGCTCGGGCGGTGGTGGGTCGGGTGGCGGCGGATCTGATATC
GTCATGACTCAAGCCGCCTTCAGCACTCCAGTCACTCTCGGCACAAGCGCCAGCATCAG
CTGTAGGTCCAGCCAGTCTCTGCTGCACAGCAACGGCATCACTTATCTGTACTGGTATCT
GCAGAAGCCCGGCCAAAGCCCACAGCTGCTGATCTACCAGATGAGCAATCTGGCCAGC
GGCGTGCCAGATAGATTCAGCAGCAGCGGAAGCGGAACAGACTTCACACTGAGGATCT
CTAGGGTGGAAGCCGAAGATGTGGGCGTCTATTACTGCGCCCAGAACCTCGAGTTCCCT
TTCACATTCGGCAGCGGCACTAAGCTGGAGATCAAG.
(SEQ ID NO: 138)
CAGATCGTGCTGACACAGAGCCCAGCTCTGATGAGCGCCAGCCCCGGCGAGAA
GGTCACAATGACTTGCAGCGCCACATCCAGCGTGAGCTACATCTACTGGTACCAGCAGA
AGCCTAGGAGCAGCCCTAAGCCTTGGATCTACCTCACAAGCAATCTGGCCAGCGGAGTG
CCAGCTAGGTTCAGCGGAAGCGGCAGCGGCACAAGCTACTCTCTGACAATCTCCAGCAT
GGAAGCCGAAGATGCCGCCACTTACTACTGCCAGCAGTGGAGCAGCAATCCACCTACAT
TCGGAGGCGGCACTAAGCTGGAGATCAAGGGTGGCGGTGGCTCGGGCGGTGGTGGGTC
GGGTGGCGGCGGATCTGAGGTGCAGCTCCAACAGAGCGGACCAGAGCTGGTGAAACC
CGGCGCCAGCATGAAGATCAGCTGTAAGGCCTCCGGCTACAGCTTCACTGGCTACACTA
TGAACTGGGTGAAGCAGTCCCACGGCAAGAATCTGGAGTGGATCGGACTGATCAACCC
ATACAACGGCGGCACAAACTACAACCAGAAGTTCAAGGGCAAGGCCACTCTGACAGTC
GATAAGAGCTCCAGCACTGCCTACATGGAGCTGCTGAGCCTCACTAGCGAGGACAGCGC
TGTGTACTACTGTGCCTTCTCCTACTACGGCTCTAGGGGCTTCTACTTCGATTACTGGGGC
CAAGGCACAACACTGACAGTGTCCAGC.
(SEQ ID NO: 139)
GAGGTGCAGCTCCAACAGAGCGGACCAGAGCTGGTGAAACCCGGCGCCAGCA
TGAAGATCAGCTGTAAGGCCTCCGGCTACAGCTTCACTGGCTACACTATGAACTGGGTG
AAGCAGTCCCACGGCAAGAATCTGGAGTGGATCGGACTGATCAACCCATACAACGGCG
GCACAAACTACAACCAGAAGTTCAAGGGCAAGGCCACTCTGACAGTCGATAAGAGCTC
CAGCACTGCCTACATGGAGCTGCTGAGCCTCACTAGCGAGGACAGCGCTGTGTACTACT
GTGCCTTCTCCTACTACGGCTCTAGGGGCTTCTACTTCGATTACTGGGGCCAAGGCACAA
CACTGACAGTGTCCAGCGGTGGCGGTGGCTCGGGCGGTGGTGGGTCGGGTGGCGGCGG
ATCTCAGATCGTGCTGACACAGAGCCCAGCTCTGATGAGCGCCAGCCCCGGCGAGAAG
GTCACAATGACTTGCAGCGCCACATCCAGCGTGAGCTACATCTACTGGTACCAGCAGAA
GCCTAGGAGCAGCCCTAAGCCTTGGATCTACCTCACAAGCAATCTGGCCAGCGGAGTGC
CAGCTAGGTTCAGCGGAAGCGGCAGCGGCACAAGCTACTCTCTGACAATCTCCAGCATG
GAAGCCGAAGATGCCGCCACTTACTACTGCCAGCAGTGGAGCAGCAATCCACCTACATT
CGGAGGCGGCACTAAGCTGGAGATCAAG.
(SEQ ID NO: 140)
GATATCGTGATGACTCAGTCCCCAGCCATCATGTCCGCCAGCCCCGGCGAGAAG
GTGACTATGACTTGCTCCGCCAGCAGCAGCGTGAGCTACATGCACTGGTACCAGCAGAA
GAGCGGCACATCCCCAAAGAGGTGGATCTACGACACAAGCAAGCTGGCCAGCGGCGTG
CCAGCCAGATTCAGCGGCTCCGGCAGCGGAACAAGCTACTCTCTGACTATCAGCAGCAT
GGAGACAGAGGACGCTGCCACTTACTACTGCCAGCAGTGGAGCAGCAATCCTCCAACT
TTCGGCGGAGGCACTAAGCTGGAGCTGAAGGGTGGCGGTGGCTCGGGCGGTGGTGGGT
CGGGTGGCGGCGGATCTGAAGTGCAGCTGCAACAGAGCGGCCCAGAGCTCGTGAAGCC
CGGCGCCAGCATGAAGATCAGCTGCAAGGCCAGCGGCTACAGCTTCACTGGCTACACA
ATGAACTGGGTCAAGCAGAGCCACGGAAAAAATCTGGAGTGGATCGGACTGATCAACC
CTTACAACGGCGGCACAAGGTACAATCAGAAGTTCAAGGGCAAGGCCACTCTCACTGT
GGATAAGAGCAGCAGCACTGCCTACATGGAGCTGCTGTCTCTGACAAGCGAGGATAGCG
CCGTGTACTACTGCGCCAGCTCCTCCTATAGGAACGACGGCAACTGGTACTTCGATGTGT
GGGGCGCCGGCACTACTGTGACAGTGAGCTCC.
(SEQ ID NO: 141)
GAAGTGCAGCTGCAACAGAGCGGCCCAGAGCTCGTGAAGCCCGGCGCCAGCA
TGAAGATCAGCTGCAAGGCCAGCGGCTACAGCTTCACTGGCTACACAATGAACTGGGTC
AAGCAGAGCCACGGAAAAAATCTGGAGTGGATCGGACTGATCAACCCTTACAACGGCG
GCACAAGGTACAATCAGAAGTTCAAGGGCAAGGCCACTCTCACTGTGGATAAGAGCAG
CAGCACTGCCTACATGGAGCTGCTGTCTCTGACAAGCGAGGATAGCGCCGTGTACTACT
GCGCCAGCTCCTCCTATAGGAACGACGGCAACTGGTACTTCGATGTGTGGGGCGCCGGC
ACTACTGTGACAGTGAGCTCCGGTGGCGGTGGCTCGGGCGGTGGTGGGTCGGGTGGCG
GCGGATCTGATATCGTGATGACTCAGTCCCCAGCCATCATGTCCGCCAGCCCCGGCGAG
AAGGTGACTATGACTTGCTCCGCCAGCAGCAGCGTGAGCTACATGCACTGGTACCAGCA
GAAGAGCGGCACATCCCCAAAGAGGTGGATCTACGACACAAGCAAGCTGGCCAGCGGC
GTGCCAGCCAGATTCAGCGGCTCCGGCAGCGGAACAAGCTACTCTCTGACTATCAGCAG
CATGGAGACAGAGGACGCTGCCACTTACTACTGCCAGCAGTGGAGCAGCAATCCTCCA
ACTTTCGGCGGAGGCACTAAGCTGGAGCTGAAG.
(SEQ ID NO: 142)
CAGATTGTGCTGTCCCAGTCCCCAGCCATTCTGAGCGCCAGCCCCGGCGAGAA
GGTGACTATGACTTGTAGGGCCACAAGCAGCGTGAGCTACATGTACTGGTACCAGCAGA
AGCCCGGCAGCAGCCCTAAGCCTTGGATCTACGCCACAAGCAATCTGGCCAGCGGCGTC
CCAGCTAGATTTAGCGGCAGCGGATCCGGCACTAGCTATTCTCTGACTATCTCTAGGGTC
GAGGCCGAAGATGCCGCCACATACTACTGCCAGCAGTGGTCCTCCAATCCTCCAACATT
CGGCGGCGGAACTAAGCTGGAGAAGAAGGGTGGCGGTGGCTCGGGCGGTGGTGGGTC
GGGTGGCGGCGGATCTGAAGTGCAGCTGCAGCAGAGCGGACCAGAGCTGGTCAAGCC
CGGCGCCAGCATGAAGATCAGCTGTAAGGCCAGCGGCTACAGCATCACTGGCTACACTA
TGAACTGGGTGAAGCAGAGCCACGGCAAGAACCTCGAGTGGATTGGCCTCGTGAACCC
ATACAACGGCGGCACTTCCTACAACCAGAAGTTCAAAGGCAAGGCCACACTCACAGTC
GATAAGTCCAGCTCCACAGCCTACATGGAGCTGCTGTCTCTGAAGAGCGAGGATAGCGC
TGTCTACTACTGTGCCATCAGCAGATACGGCAGCGAGAGCTGGTACTTCGACGTGTGGG
GCGCCGGCACAACAGTGACAGTGAGCAGC.
(SEQ ID NO: 143)
GAAGTGCAGCTGCAGCAGAGCGGACCAGAGCTGGTCAAGCCCGGCGCCAGCA
TGAAGATCAGCTGTAAGGCCAGCGGCTACAGCATCACTGGCTACACTATGAACTGGGTG
AAGCAGAGCCACGGCAAGAACCTCGAGTGGATTGGCCTCGTGAACCCATACAACGGCG
GCACTTCCTACAACCAGAAGTTCAAAGGCAAGGCCACACTCACAGTCGATAAGTCCAG
CTCCACAGCCTACATGGAGCTGCTGTCTCTGAAGAGCGAGGATAGCGCTGTCTACTACT
GTGCCATCAGCAGATACGGCAGCGAGAGCTGGTACTTCGACGTGTGGGGCGCCGGCAC
AACAGTGACAGTGAGCAGCGGTGGCGGTGGCTCGGGCGGTGGTGGGTCGGGTGGCGG
CGGATCTCAGATTGTGCTGTCCCAGTCCCCAGCCATTCTGAGCGCCAGCCCCGGCGAGA
AGGTGACTATGACTTGTAGGGCCACAAGCAGCGTGAGCTACATGTACTGGTACCAGCAG
AAGCCCGGCAGCAGCCCTAAGCCTTGGATCTACGCCACAAGCAATCTGGCCAGCGGCG
TCCCAGCTAGATTTAGCGGCAGCGGATCCGGCACTAGCTATTCTCTGACTATCTCTAGGG
TCGAGGCCGAAGATGCCGCCACATACTACTGCCAGCAGTGGTCCTCCAATCCTCCAACA
TTCGGCGGCGGAACTAAGCTGGAGAAGAAG.
(SEQ ID NO: 144)
GATATCGTGCTGACTCAGAGCCCAGCCTCTCTGGTGGTGTCTCTGGGACAGAGG
GCCACAATCAGCTGTAGGACTTCCAAGAGCGTGAGCAGCTCCGCCTACAGCTACATGCA
CTGGTACCAGCAGAAGCCCGGCCAGCCTCCTAAGGTGCTGATCTATCTGGCCAGCAATC
TGGAGAGCGGCGTCCCAGCTAGATTCAGCGGCTCCGGAAGCGGCACTGACTTCACTCT
GAACATCCACCCAGTGGAAGAGGAGGATGCCGCCACATACTACTGCCAGCACTCTAGGG
AGCTGCCTTTCACATTTGGCAGCGGAACTAAGCTGGAGATCAAGGGTGGCGGTGGCTCG
GGCGGTGGTGGGTCGGGTGGCGGCGGATCTCAAGTGCATCTGCAGCAGAGCGGCGCTG
AGCTGATGAAGCCCGGCGCCAGCGTGAAGATTAGCTGCAAGGCCACTGGCTACACATTC
AACATCTACTGGATCGACTGGGTGAAGCAGAGGCCCGGCCACGGACTGGAATGGATCG
GCGAAATTCTGCCCGGCAGCGGCAACACTCACTACAACGAGAACTTCAAGGGCAAGGC
CACAATGACAGCCGACACAAGCTCCAACACTGCTTACATGCAGCTGACTTCTCTGACTA
GCGAGGACAGCGCCGTGTACTATTGCGCTAGGACAGACGGAAGGGGCTACTTCGATTAC
TGGGGCCAAGGCACTACACTCACAGTGAGCAGC.
(SEQ ID NO: 145)
CAAGTGCATCTGCAGCAGAGCGGCGCTGAGCTGATGAAGCCCGGCGCCAGCGT
GAAGATTAGCTGCAAGGCCACTGGCTACACATTCAACATCTACTGGATCGACTGGGTGA
AGCAGAGGCCCGGCCACGGACTGGAATGGATCGGCGAAATTCTGCCCGGCAGCGGCAA
CACTCACTACAACGAGAACTTCAAGGGCAAGGCCACAATGACAGCCGACACAAGCTCC
AACACTGCTTACATGCAGCTGACTTCTCTGACTAGCGAGGACAGCGCCGTGTACTATTG
CGCTAGGACAGACGGAAGGGGCTACTTCGATTACTGGGGCCAAGGCACTACACTCACA
GTGAGCAGCGGTGGCGGTGGCTCGGGCGGTGGTGGGTCGGGTGGCGGCGGATCTGATA
TCGTGCTGACTCAGAGCCCAGCCTCTCTGGTGGTGTCTCTGGGACAGAGGGCCACAATC
AGCTGTAGGACTTCCAAGAGCGTGAGCAGCTCCGCCTACAGCTACATGCACTGGTACCA
GCAGAAGCCCGGCCAGCCTCCTAAGGTGCTGATCTATCTGGCCAGCAATCTGGAGAGCG
GCGTCCCAGCTAGATTCAGCGGCTCCGGAAGCGGCACTGACTTCACTCTGAACATCCAC
CCAGTGGAAGAGGAGGATGCCGCCACATACTACTGCCAGCACTCTAGGGAGCTGCCTTT
CACATTTGGCAGCGGAACTAAGCTGGAGATCAAG.
(SEQ ID NO: 146)
CAAATTGTTCTCACACAGAGCCCTCCTATCATGAGCGCCAGCCCCGGCGAGAAG
GTGACTATGACTTGTTCCGCCAGCAGCAACATCAGCTACATGCACTGGTACCAGCAGAA
GTCCGGCACAAGCCCAAAGAGGTGGATCTACGACACAAGCAAGCTGGCCAGCGGCGTG
CCAGCCAGATTTAGCGGCTCCGGAAGCGGCACTAGCTACTCTCTGACAATCAGCAGCAT
GGAAGCCGAGGACGCCGCTACATACTACTGCCAGCAGTGGAGCTCCGTCCCACTGACTT
TCGGCGCTGGCACTAAGCTGGAGATCAAGGGTGGCGGTGGCTCGGGCGGTGGTGGGTC
GGGTGGCGGCGGATCTCAGGTTCAGCTGCAGCAGTCCGGCGCTGAGCTCATGAAGCCC
GGCGCCAGCGTGAAGATCAGCTGCAAGGCCACTGGCTACACATTCAGCAGCTACTGGAT
CGAGTGGGTGAAGCAGAGACCCGGCCACGGACTGGAATGGCTGGGAGAGTTTCTGCCT
AGAAGCGGCAAGACAAACTACAACGAGGAGTTTAGGGGCAAGGCTACATTCACTGCCG
ACACATCCAGCAACACTGCCTACATGCAGCTGAGCAGCCTCACAAGCGAGGATTCCGCC
GTCTACTACTGTGCTAGGACTGATCCTCCTTACTTCGGAGTGTGGGGCGCTGGCACAATG
GTGGCTGTGAGCAGC.
(SEQ ID NO: 147)
CAGGTTCAGCTGCAGCAGTCCGGCGCTGAGCTCATGAAGCCCGGCGCCAGCGT
GAAGATCAGCTGCAAGGCCACTGGCTACACATTCAGCAGCTACTGGATCGAGTGGGTGA
AGCAGAGACCCGGCCACGGACTGGAATGGCTGGGAGAGTTTCTGCCTAGAAGCGGCAA
GACAAACTACAACGAGGAGTTTAGGGGCAAGGCTACATTCACTGCCGACACATCCAGC
AACACTGCCTACATGCAGCTGAGCAGCCTCACAAGCGAGGATTCCGCCGTCTACTACTG
TGCTAGGACTGATCCTCCTTACTTCGGAGTGTGGGGCGCTGGCACAATGGTGGCTGTGA
GCAGCGGTGGCGGTGGCTCGGGCGGTGGTGGGTCGGGTGGCGGCGGATCTCAAATTGT
TCTCACACAGAGCCCTCCTATCATGAGCGCCAGCCCCGGCGAGAAGGTGACTATGACTT
GTTCCGCCAGCAGCAACATCAGCTACATGCACTGGTACCAGCAGAAGTCCGGCACAAG
CCCAAAGAGGTGGATCTACGACACAAGCAAGCTGGCCAGCGGCGTGCCAGCCAGATTT
AGCGGCTCCGGAAGCGGCACTAGCTACTCTCTGACAATCAGCAGCATGGAAGCCGAGG
ACGCCGCTACATACTACTGCCAGCAGTGGAGCTCCGTCCCACTGACTTTCGGCGCTGGC
ACTAAGCTGGAGATCAAG.

Wherein, in the present application, the nucleotide sequences shown in the above SEQ ID NO: 112 and 121 encode the heavy and light chains of the 2008 chimeric antibody, respectively; the nucleotide sequences shown in the above SEQ ID NO: 113 and 122 encode the heavy and light chains of the 23E12 chimeric antibody, respectively; the nucleotide sequences shown in the above SEQ ID NO: 114 and 123 encode the heavy and light chains of the 27H3 chimeric antibody, respectively; the nucleotide sequences shown in the above SEQ ID NO: 115 and 124 encode the heavy and light chains of the 21E5 chimeric antibody, respectively; the nucleotide sequences shown in the above SEQ ID NO: 116 and 125 encode the heavy and light chains of the 2A5 chimeric antibody, respectively; the nucleotide sequences shown in the above SEQ ID NO: 117 and 126 encode the heavy and light chains of the 4H4 chimeric antibody, respectively; the nucleotide sequences shown in the above SEQ ID NO: 118 and 127 encode the heavy and light chains of the 22D12 chimeric antibody, respectively; the nucleotide sequences shown in the above SEQ ID NO: 119 and 128 encode the heavy and light chains of the 1B9 chimeric antibody, respectively; the nucleotide sequences shown in the above SEQ ID NO: 120 and 129 encode the heavy and light chains of the 15D4 chimeric antibody, respectively. The nucleotide sequences shown in SEQ ID NO: 130, 132, 134, 136, 138, 140, 142, 144, and 146 encode single chain antibody fragment of nucleotide sequences shown in SEQ ID NO: 94, 96, 98, 100, 102, 104, 106, 108, and 110, respectively; the nucleotide sequences shown in SEQ ID NO: 131, 133, 135, 137, 139, 141, 143, 145, and 147 encode single chain antibody fragment of nucleotide sequences shown in SEQ ID NO: 95, 97, 99, 101, 103, 105, 107, 109 and 111, respectively.

In a third aspect of the invention, the invention provides an expression vector. According to the embodiment of the present invention, the expression vector carries the aforementioned nucleic acid molecule. After the expression vector according to the embodiment of the present invention is introduced into a suitable recipient cell, it can effectively realize the expression of the aforementioned antibody or antigen-binding fragment that specifically recognizes TrkA under the mediation of the regulatory system, thereby realizing that the antibody or antigen-binding fragment are obtained in large quantities in vitro.

According to the embodiment of the present invention, the expression vector may further include at least one of the following additional technical features:

according to the embodiment of the present invention, the expression vector is a eukaryotic expression vector. Thus, the expression of the antibody or antigen-binding fragment thereof that specifically recognizes TrkA as described above in eukaryotic cells, such as CHO cells, is achieved.

In a fourth aspect of the invention, the invention provides a recombinant cell. According to the embodiment of the present invention, the recombinant cell carries the aforementioned nucleic acid molecule, or expresses the aforementioned antibody or antigen-binding fragment thereof. The recombinant cells according to the embodiments of the present invention can be used for the expression and large-scale acquisition in vitro of the antibodies or antigen-binding fragments thereof that specifically recognize TrkA as described above.

According to the embodiment of the present invention, the above-mentioned recombinant cell may further include at least one of the following additional technical features:

according to the embodiment of the present invention, the recombinant cell is obtained by introducing the aforementioned expression vector into a host cell.

According to the embodiment of the present invention, the expression vector is introduced into the host cell by a method of electrotransduction.

According to the embodiment of the present invention, the recombinant cell is a eukaryotic cell.

According to the embodiment of the present invention, the recombinant cell is a mammalian cell.

In a fifth aspect of the invention, the invention provides a pharmaceutical composition. According to the embodiment of the present invention, the pharmaceutical composition contains the aforementioned antibody, the aforementioned nucleic acid molecule, the aforementioned expression vector or the aforementioned recombinant cell. The antibody or the expressed antibody contained in the pharmaceutical composition according to the embodiment of the present invention can not only specifically targeted-bind to the TrkA receptor, block the binding of NGF and TrkA, effectively inhibit pain, but also has the characteristics of low immunogenicity and almost no antibody-dependent cell-mediated cytotoxicity (ADCC).

In a sixth aspect of the present invention, the present invention provides the use of the aforementioned antibody, the aforementioned nucleic acid molecule, the aforementioned expression vector or the aforementioned recombinant cell, and the aforementioned pharmaceutical composition in the preparation of a medicament for the treatment or prevention of pain, cancer, inflammation or inflammatory diseases, neurodegenerative diseases, Sjogren's syndrome, endometriosis, diabetic peripheral neuropathy, prostatitis, pelvic pain syndrome, diseases related to the imbalance in the regulation of bone remodeling and diseases caused by abnormal signaling of connective tissue growth factor.

According to the embodiment of the present invention, the above-mentioned use may further include at least one of the following additional technical features:

according to the embodiment of the present invention, the medicament is used to treat or prevent neuropathic pain, inflammatory pain, cancer-related pain, fracture-related pain, surgery-related pain, inflammatory lung disease, interstitial cystitis, painful bladder syndrome, inflammatory bowel disease, inflammatory skin disease, Raynaud's syndrome, idiopathic pulmonary fibrosis, scar (hypertrophy, keloid type and other forms), sclerosis, endocardial myocardial fibrosis, atrial fibrosis, bone marrow fibrosis, progressive massive fibrosis (lung), renal-derived systemic fibrosis, scleroderma, systemic sclerosis, joint fibrosis, ocular fibrosis, non-small cell lung cancer, papillary thyroid cancer, glioblastoma multiforme, colorectal cancer, melanoma, bile duct cancer or sarcoma, acute myeloid leukemia, large cell neuroendocrine cancer, neuroblastoma, prostate cancer, pancreatic cancer, melanoma, head and neck squamous cell carcinoma or gastric cancer.

In a sixth aspect of the present invention, the present invention provides the aforementioned antibody, the aforementioned nucleic acid molecule, the aforementioned expression vector or the aforementioned recombinant cell, and the aforementioned pharmaceutical composition for use in the preparation of a medicament for the treatment or prevention of pain, cancer, inflammation or inflammatory diseases, neurodegenerative diseases, Sjogren's syndrome, endometriosis, diabetic peripheral neuropathy, prostatitis, pelvic pain syndrome, diseases related to the imbalance in the regulation of bone remodeling and diseases caused by abnormal signaling of connective tissue growth factor.

According to the embodiment of the present invention, the above-mentioned use may further include at least one of the following additional technical features:

according to the embodiment of the present invention, the medicament is used to treat or prevent neuropathic pain, inflammatory pain, cancer-related pain, fracture-related pain, surgery-related pain, inflammatory lung disease, interstitial cystitis, painful bladder syndrome, inflammatory bowel disease, inflammatory skin disease, Raynaud's syndrome, idiopathic pulmonary fibrosis, scar (hypertrophy, keloid type and other forms), sclerosis, endocardial myocardial fibrosis, atrial fibrosis, bone marrow fibrosis, progressive massive fibrosis (lung), renal-derived systemic fibrosis, scleroderma, systemic sclerosis, joint fibrosis, ocular fibrosis, non-small cell lung cancer, papillary thyroid cancer, glioblastoma multiforme, colorectal cancer, melanoma, bile duct cancer or sarcoma, acute myeloid leukemia, large cell neuroendocrine cancer, neuroblastoma, prostate cancer, pancreatic cancer, melanoma, head and neck squamous cell carcinoma or gastric cancer.

In a sixth aspect of the present invention, the present invention provides a method of treating or preventing pain, cancer, inflammation or inflammatory diseases, neurodegenerative diseases, Sjogren's syndrome, endometriosis, diabetic peripheral neuropathy, prostatitis, pelvic pain syndrome, diseases related to imbalance in the regulation of bone remodeling and diseases caused by abnormal signaling of connective tissue growth factor in a subject, comprising administering to a subject in need thereof a therapeutically effective amount of the aforementioned antibody, the aforementioned nucleic acid molecule, the aforementioned expression vector or the aforementioned recombinant cell, and the aforementioned pharmaceutical composition.

According to the embodiment of the present invention, the above-mentioned method may further include at least one of the following additional technical features:

according to the embodiment of the present invention, the medicament is used to treat or prevent neuropathic pain, inflammatory pain, cancer-related pain, fracture-related pain, surgery-related pain, inflammatory lung disease, interstitial cystitis, painful bladder syndrome, inflammatory bowel disease, inflammatory skin disease, Raynaud's syndrome, idiopathic pulmonary fibrosis, scar (hypertrophy, keloid type and other forms), sclerosis, endocardial myocardial fibrosis, atrial fibrosis, bone marrow fibrosis, progressive massive fibrosis (lung), renal-derived systemic fibrosis, scleroderma, systemic sclerosis, joint fibrosis, ocular fibrosis, non-small cell lung cancer, papillary thyroid cancer, glioblastoma multiforme, colorectal cancer, melanoma, bile duct cancer or sarcoma, acute myeloid leukemia, large cell neuroendocrine cancer, neuroblastoma, prostate cancer, pancreatic cancer, melanoma, head and neck squamous cell carcinoma or gastric cancer.

In a seventh aspect of the present invention, the present invention provides a kit for detecting TrkA. According to the embodiment of the present invention, the kit includes any one of the antibodies described above. The TrkA antibody described above can specifically targeted-bind to TrkA. The kit according to the embodiment of the present invention can realize the specific detection of TrkA. For example, when the antibody is combined with a fluorescent group, a fluorescence detection device can be used to realize the localization or real-time detection of TrkA.

In an eighth aspect of the present invention, the present invention provides the use of the aforementioned antibody, the aforementioned nucleic acid molecule, the aforementioned expression vector, or the aforementioned recombinant cell in the preparation of a kit for detecting TrkA or diagnosing a TrkA-related disease.

In an eighth aspect of the present invention, the present invention provides the aforementioned antibody, the aforementioned nucleic acid molecule, the aforementioned expression vector, or the aforementioned recombinant cell for use in the preparation of a kit for detecting TrkA or diagnosing a TrkA-related disease.

In an eighth aspect of the present invention, the present invention provides a method of detecting TrkA or diagnosing a TrkA-related disease in a subject comprising administering to a subject in need thereof a therapeutically effective amount of the aforementioned antibody, the aforementioned nucleic acid molecule, the aforementioned expression vector, or the aforementioned recombinant cell.

In a ninth aspect of the present invention, the present invention provides a mouse B cell. According to the embodiment of the present invention, the genome of the B cell carries a sequence encoding a constant region, and the constant region sequence has S108P, F114A, L115A, R289K mutations and 327 K deletion mutations compared to the constant region of human IgG4. The constant region of human IgG4 has the amino acid sequence shown in SEQ ID NO: 148.

(SEQ ID NO: 148)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLEPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK.

The mouse B cell according to the embodiments of the present invention can be used to secrete mouse antibodies. The immunogenicity of the secreted mouse antibodies is further reduced in humans, the stability is significantly improved, and the half-life in vivo is significantly extended.

In a tenth aspect of the present invention, the present invention provides the use of the mouse B cell described above in the preparation of monoclonal antibodies.

In a tenth aspect of the present invention, the present invention provides the mouse B cell described above for use in the preparation of monoclonal antibodies.

In a tenth aspect of the present invention, the present invention provides a method of detecting TrkA or diagnosing a TrkA-related disease comprising culturing the mouse B cell described above so that the monoclonal antibody is produced.

### BRIEF DESCRIPTION OF THE DRAWINGS

FIG. 1 is an experimental result diagram of nine monoclonal hybridoma cell lines capable of producing anti-TrkA monoclonal antibodies that block the binding of NGF and TrkA at the molecular level, obtained by screening by ELISA method according to the embodiment of the present invention;

FIG. 2 is an experimental result diagram of nine monoclonal hybridoma cell lines capable of producing anti-TrkA monoclonal antibodies that block the binding of NGF and TrkA at the cell level, obtained by flow screening according to the embodiment of the present invention, % parent (percentage);

FIG. 3 is an experimental result diagram of the detection of the binding of the monoclonal antibody produced by each positive clone supernatant to the Mouse-TrKA receptor according to the ELISA method according to the embodiment of the present invention;

FIG. 4 is an experimental result diagram of the evaluation of blocking activity of test antibodies using HEK-293T-TrkA cell model according to the embodiment of the present invention, % parent (percentage);

FIG. 5 is an experimental result diagram of Tanezumab used to verify whether the NIH-3T3-TrkA cell model can be used to evaluate the activity of a drug (test drug) in vitro that inhibits the NGF-TrkA pathway according to the embodiment of the present invention;

FIG. 6 is an experimental result diagram of the tyrosine phosphorylation level of TrkA protein under the action of the test antibody and the positive antibody MNAC13 by the AlphaLISA method according to an embodiment of the invention;

FIG. 7 is a result diagram of detecting an affinity EC₅₀ of a test antibody by flow cytometry according to the embodiment of the present invention;

FIG. 8A-8G is a result diagram of evaluating the specificity of the binding of a test antibody to a target TrKA by an ELISA method according to the embodiment of the present invention;

FIG. 9 is a result diagram of detecting the binding ability of the test antibody to the Mouse-TrKA protein by applying an ELISA method according to the embodiment of the present invention;

FIG. 10 is a result diagram of detecting the binding ability of the test antibody to the Mouse-TrKA protein by flow cytometry according to the embodiment of the present invention;

FIG. 11 is a result diagram of detecting the inhibitory effect of the test antibody on the binding of Mouse-NGF and Mouse-TrKA by applying an ELISA method according to the embodiment of the present invention;

FIG. 12 is a result diagram of detecting the inhibitory effect of the test antibody on the binding of Mouse-NGF and Mouse-TrKA by flow cytometry according to the embodiment of the present invention;

FIG. 13 is a result diagram of detecting the inhibitory effect of the test antibody on the binding of Mouse-NGF and Mouse-TrKA by applying an ELISA method according to the embodiment of the present invention;

FIG. 14 is a result diagram of evaluating the analgesic activity of a test antibody in vivo using a formalin pain model according to the embodiment of the present invention; and

FIG. 15 is a result diagram of evaluating the analgesic activity of a test antibody in vivo using a Freund's adjuvant-induced inflammation and pain model according to the embodiment of the present invention.

### SPECIFIC IMPLEMENTATION

The embodiments of the present invention are described in detail below. Examples of the embodiments are shown in the accompanying drawings, wherein the same or similar reference numerals represent the same or similar elements or elements having the same or similar functions throughout. The embodiments described below with reference to the drawings are exemplary and are intended to explain the present invention, but should not be construed as limiting the present invention.

In the course of describing the present invention, the terms used herein are explained. These explanations are only for the convenience of understanding the scheme, and should not be regarded as limiting the protection scheme of the present invention.

Antibody

As used herein, the term “antibody” is an immunoglobulin molecule capable of binding to a specific antigen. It consists of two lighter molecular weight light chains and two heavier molecular weight heavy chains. The heavy (H) and light (L) chains are linked by disulfide bonds to form a tetrapeptide chain molecule. Among them, the amino acid sequence of the amino terminal (N-terminal) of the peptide chain changes greatly, which is called the variable region (V region). The carboxyl terminal (C-terminal) is relatively stable with little change, which is called the constant region (C region). The V regions of the L and H chains are referred to as VL and VH, respectively.

Some regions in the variable region have a higher degree of change in amino acid composition and arrangement order. They are called hypervariable regions (HVR). Hypervariable regions are where antigens and antibodies bind, so they are also called complementarity-determining region (CDR). There are three CDRs on both the heavy and light chain variable regions.

The present invention utilizes the extracellular segment of TrkA to obtain highly specific and high affinity anti-TrkA Fab (antigen-binding fragment) antibody fragments through immunization. The antibody fragment can specifically bind to the TrkA antigen, and thus can be used for targeted treatment of diseases such as pain or tumor.

In some embodiments, the invention provides an antibody or antigen-binding fragment capable of specifically recognizing TrkA, wherein the antibody comprises a CDR sequence selected from at least one of the following or an amino acid sequence having at least 95% identity with it: heavy chain variable region CDR sequences: SEQ ID NO: 1˜27, light chain variable region CDR sequences: SEQ IN NO: 28˜54. In other embodiments, the antibodies or antigen-binding fragments provided by the present invention have conservative amino acid substitutions compared to the above heavy and light chains. “Antigen-binding fragment” refers to an antibody fragment that retains the ability to specifically bind to an antigen (ROR2). “Conservative amino acid substitution” refers to the replacement of an amino acid with a residue that is biologically, chemically, or structurally similar to another amino acid. Biological similarity means that the substitution does not destroy the TrkA antibody or biological activity with the TrkA antigen. Structural similarity refers to side chains with similar lengths of amino acids, such as alanine, glycine, or serine, or side chains of similar size. Chemical similarity means that amino acids have the same charge or are both hydrophilic or hydrophobic. For example, the hydrophobic residues isoleucine, valine, leucine or methionine are substituted with each other. Alternatively, polar amino acid is substituted with another polar amino acid, such as lysine is substituted with arginine, aspartic acid is substituted with glutamic acid, asparagine is substituted with glutamine, threonine is substituted with serine, etc.

In some embodiments, the present invention provides an antibody or antigen-binding fragment, wherein the antibody or antigen-binding fragment has a heavy chain variable region of the amino acid sequence shown in any one of SEQ ID NO: 55˜63 and has a light chain variable region of the amino acid sequence shown in any one of SEQ ID NO: 64˜72. The inventors can obtain the CDR regions of the above-mentioned anti-heavy chain variable region sequences (as shown in SEQ ID NO: 1˜27) and the CDR regions of the light chain variable region sequence (such as SEQ ID NO: 28˜54) through an antibody sequence alignment database (NCBI, IMGT). In other embodiments, the heavy chain variable region sequence of the antibody or antigen-binding fragment has conservative amino acid substitutions compared to the amino acid sequence shown in SEQ ID NO: 55˜63. In some embodiments, the light chain variable region sequence of the antibody or antigen-binding fragment has conservative amino acid substitutions compared to the amino acid sequence shown in any one of SEQ ID NO: 64˜72. Of course, these conservative amino acid substitutions will not alter the biological function of the antibody or antigen-binding fragment. In some specific ways, these conservative amino acid substitutions can occur on amino acids other than the CDR regions in the heavy and light chain variable regions.

In some preferred embodiments, the invention provides an anti-TrkA antibody having a heavy chain of the amino acid sequence shown in any one of SEQ ID NO: 76˜84 and having a light chain of the amino acid sequence shown in any one of SEQ ID NO: 85˜93.

In some preferred embodiments, the present invention provides an anti-TrkA single chain fragment antibody, wherein the antibody has the amino acid sequence shown in SEQ ID NO: 94˜111.

Nucleic Acid Molecule, Expression Vector, Recombinant Cell

In the process of preparing or obtaining these antibodies, nucleic acid molecules expressing these antibodies can be connected to different vectors and then express in different cells to obtain corresponding antibodies.

To this end, the present invention also provides an isolated nucleic acid molecule, which encodes the antibody or antigen-binding fragment described above.

In some embodiments, the isolated nucleic acid molecule has a nucleotide sequence shown in any one of SEQ ID NO: 112˜120 or has a nucleotide sequence shown in any one of SEQ ID NO: 121˜129 or has a nucleotide sequence as shown in any one of SEQ ID NO: 130˜147.

In some embodiments, the isolated nucleic acid molecule has at least 90% homology with the nucleotide sequence shown in the above SEQ ID NO: 112˜120, and preferably has at least 95% homology, and more preferably has at least 98%, 99% homology. In at least some embodiments, the isolated polynucleotide has at least 90% homology with the nucleotide sequence shown in SEQ ID NO: 121˜129, and preferably has at least 95% homology, and more preferably has at least 98%, 99% homology. In at least some embodiments, the isolated polynucleotide has at least 90% homology with the nucleotide sequence shown in SEQ ID NO: 130˜147, and preferably has at least 95% homology, and more preferably has at least 98%, 99% homology. These sequences which are homologous to the nucleotide sequences shown in SEQ ID NO: 112˜120 or SEQ ID NO: 121˜129 or SEQ ID NO: 130˜147 can express amino acids similar to SEQ ID NO: 76˜84 or SEQ ID NO: 85˜93 or SEQ ID NO: 94·111, so that they can specifically bind to the TrkA antigen and achieve the targeted function of antibodies.

In some preferred embodiments, the isolated nucleic acid molecule has a heavy chain nucleotide sequence shown in SEQ ID NO: 112˜120 and a light chain nucleotide sequence shown in SEQ ID NO: 121˜129. These nucleotide sequences are optimized for species and are more easily expressing in mammalian cells.

The present invention also provides an expression vector, wherein the expression vector comprises the aforementioned isolated nucleic acid molecule. When the aforementioned isolated polynucleotide is ligated to a vector, the polynucleotide can be directly or indirectly connected to control elements on the vector, as long as these control elements can control the translation and expression of the polynucleotide. Of course, these control elements can come directly from the vector itself, or they can be exogenous, that is, not from the vector itself. Of course, the polynucleotide may be operably linked to the control element. “Operably linked” herein refers to the connection of an exogenous gene to a vector, so that control elements in the vector, such as transcription control sequences and translation control sequences, can exert its expected function of regulating the transcription and translation of exogenous genes. Of course, the polynucleotides used to encode the heavy and light chains of the antibodies can be inserted into different vectors independently, and they are usually inserted into the same vector. Commonly used vectors can be, for example, plasmids, phages, and the like. For example Plasmid-X plasmid.

The invention also provides a recombinant cell, which contains the expression vector. The expression vector can be introduced into mammalian cells, and the recombinant cells are constructed and obtained, and then these recombinant cells can be used to express the antibodies or antigen-binding fragments provided by the present invention. By culturing the recombinant cells, corresponding antibodies can be obtained. These usable mammalian cells may be, for example, CHO cells and the like.

Pharmaceutical Composition, Kit and Pharmaceutical Uses and Uses in the Preparation of Kits

The invention also provides a pharmaceutical composition, which comprises the antibody or antigen-binding fragment described above and a pharmaceutically acceptable carrier.

The anti-TrkA antibodies provided herein can be incorporated into a pharmaceutical composition suitable for administration to a subject. Generally, these pharmaceutical compositions include the anti-TrkA antibodies provided herein as well as a pharmaceutically acceptable carrier. A “pharmaceutically acceptable carrier” may include any and all solvents, dispersion media, coatings, antibacterial and antifungal agents, isotonic and delayed absorption agents, and the like that are physiologically compatible. Specific examples may be one or more of water, saline, phosphate buffered saline, glucose, glycerol, ethanol, and the like, and combinations thereof. In many cases, pharmaceutical compositions include isotonic agents, such as sugars, polyalcohols (such as mannitol, sorbitol), or sodium chloride. Of course, pharmaceutically acceptable carriers may also include minor amounts of auxiliary substances, such as wetting or emulsifying agents, preservatives or buffering agents, to extend the shelf life or efficacy of the antibody.

For example, the antibodies of the invention can be incorporated into pharmaceutical compositions suitable for parenteral administration (e.g., intravenous, subcutaneous, intraperitoneal, intramuscular). These pharmaceutical compositions can be prepared in various forms. Examples are liquid, semi-solid, and solid dosage forms, including, but not limited to, liquid solutions (e.g., injection solutions and infusion solutions), dispersing or suspending agents, tablets, pills, powders, liposomes, and suppositories. Typical pharmaceutical compositions are in the form of injection solutions or infusion solutions. The antibodies can be administered by intravenous infusion or injection or intramuscular or subcutaneous injection.

Of course, the anti-TrkA antibodies herein can also be made into kits or part of other diagnostic reagents as needed. According to the embodiment of the present invention, the present invention also provides a kit comprising the above-mentioned TrkA antibody. The kit provided by the present invention can be used, for example, for detection by immunoblotting, immunoprecipitation, etc., which involve using the specific binding properties of TrkA antigen and antibodies. These kits may include any one or more of the following: antagonists, anti-TrkA antibodies or drug reference materials; protein purification columns; immunoglobulin affinity purification buffers; cell assay diluents; instructions or literature, etc. Anti-TrkA antibodies can be used for different types of diagnostic tests, such as the detection of various diseases or the presence of drugs, toxins or other proteins in vitro or in vivo. For example, it can be used to test related diseases by detecting the serum or blood of the subject. Such related diseases may include TrkA-related diseases such as pain, cancer, inflammation or inflammatory diseases, neurodegenerative diseases, Sjogren's syndrome, endometriosis, diabetic peripheral neuropathy, prostatitis, pelvic pain syndrome, diseases related to the imbalance in regulation of bone remodeling and diseases caused by abnormal signaling of connective tissue growth factor, and the like. Of course, the antibodies provided herein can also be used for radioimmunodetection and radioimmunotherapy of the above diseases.

Specifically, the aforementioned pain, inflammation or inflammatory disease, neurodegenerative diseases, Sjogren's syndrome, endometriosis, diabetic peripheral neuropathy, prostatitis, pelvic pain syndrome, diseases related to the imbalance in regulation of bone remodeling and diseases caused by abnormal signaling of connective tissue growth factor include neuropathic pain, inflammatory pain, cancer-related pain, fracture-related pain, surgery-related pain, inflammatory lung disease, interstitial cystitis, painful bladder syndrome, inflammatory bowel disease, inflammatory skin disease, Raynaud's syndrome, idiopathic pulmonary fibrosis, scar (hypertrophy, keloid type and other forms), sclerosis, endocardial myocardial fibrosis, atrial fibrosis, bone marrow fibrosis, progressive massive fibrosis (lung), renal-derived systemic fibrosis, scleroderma, systemic sclerosis, joint fibrosis, ocular fibrosis.

These cancers or tumors can be any unregulated cell growth. Specifically, it may be non-small cell lung cancer, papillary thyroid cancer, glioblastoma multiforme, colorectal cancer, melanoma, bile duct cancer or sarcoma, acute myeloid leukemia, large cell neuroendocrine cancer, neuroblastoma, prostate cancer, pancreatic cancer, melanoma, head and neck squamous cell carcinoma or gastric cancer, etc.

When using the anti-TrkA antibody provided by the present invention to treat the above-mentioned diseases, the anti-TrkA antibody provided by the present invention may be provided to a subject. To this end, the present invention provides a method for treating the above-mentioned diseases, which comprises administering an antibody or an antigen-binding fragment thereof provided by the present invention to a subject in need.

The solution of the present invention will be explained below with reference to the examples. Those skilled in the art will understand that the following examples are only used to illustrate the present invention, and should not be regarded as limiting the scope of the present invention. If the specific technology or condition is not indicated in the examples, the technology or condition described in the literature in the art or the product description is performed. If the reagents or instruments used are not specified by the manufacturer, they are all conventional products that are commercially available.

### Example 1 Screening of Anti-TrkA Monoclonal Antibodies by Hybridoma Technology

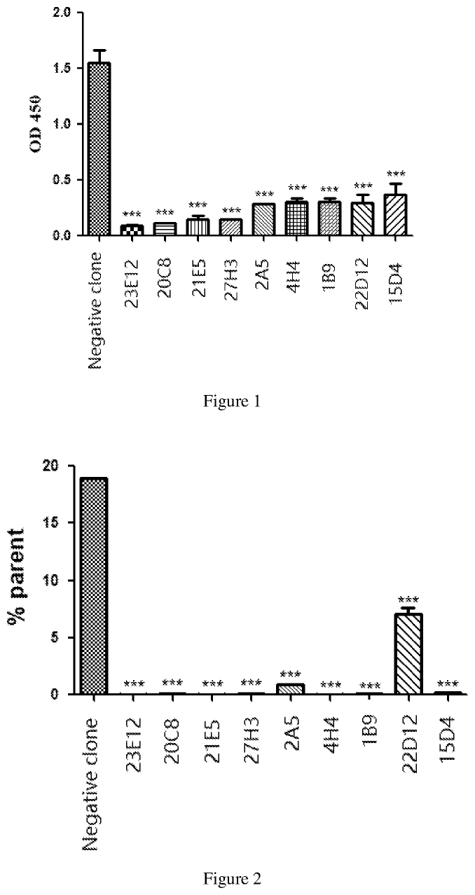

The hybridoma technology and ELISA method were used to screen hybridoma cell lines that could produce anti-TrkA monoclonal antibodies that block the binding of NGF and TrkA at the molecular level. The NCBI database was used to design genes encoding the extracellular region of TrkA protein which are constructed into mammalian eukaryotic expression system. The extracellular region of TrkA protein was expressed and used for screening of anti-TrkA monoclonal antibodies. The extracellular region of the immunogen TrkA protein was derived from Beijing Yiqiao Shenzhou Biotechnology Co., Ltd. After Balb/C mice were immunized three times by intraperitoneal injection, the serum antibody titer of the immunized mice was determined by ELISA method, reaching 10⁵. After spleen injection boosted immunity, it was fused with myeloma cells. HAT selection medium and HT selection medium were used for selective culture and screen fused hybridoma cell lines, ELISA method was used to screen positive hybridoma cell lines capable of producing anti-TrkA antibodies, and clonal culture of positive hybridoma cells was performed by limiting dilution method, then stable hybridoma cell lines capable of producing anti-TrkA monoclonal antibodies were selected. After three cloning cultures and four ELISA tests, a total of 147 positive monoclonal hybridoma cell lines were screened. NGF was biotinylated. NGF could bind to the extracellular region of TrkA protein, and anti-TrkA monoclonal antibodies could also bind to the extracellular region of TrkA protein. A competitive experiment was designed to detect the binding of NGF to the extracellular region of the TrkA protein under the action of anti-TrkA monoclonal antibodies by ELISA, and to screen anti-TrkA monoclonal antibodies that block the binding of NGF and TrkA at the molecular level. FIG. 1 shows the results of blocking experiments of 9 hybridoma cell lines that can produce anti-TrkA monoclonal antibodies that block the binding of NGF and TrkA at the molecular level among 147 positive monoclonal hybridoma cell lines, i.e., a total of 9 monoclonal hybridoma cell lines capable of producing anti-TrkA monoclonal antibodies that block the binding of NGF and TrkA at the molecular level are obtained. The experimental results are shown in FIG. 1. In the FIG. 1, OD₄₅₀ reflects the NGF signal that binds to the extracellular region of TrkA protein. The higher the reading, the stronger the NGF signal that binds to the extracellular region of TrkA protein, and the less effective the antibody in blocking the binding of NGF to TrkA; as shown in the FIG. 1, compared with the negative clone group, the ELISA readings of the 9 positive clone groups are significantly reduced, and the NGF signal binding to the extracellular region of the TrkA protein is significantly reduced; it can be seen that these 9 positive clones can reduce the NGF signal binding to the extracellular region of TrkA protein by generating anti-TrkA monoclonal antibodies that block the binding of NGF to the extracellular region of TrkA protein.

### Example 2 Screening of Anti-TrkA Monoclonal Antibodies by HEK-293T-TrkA Cell Model

HEK-293T-TrkA cell model was constructed using lentivirus technology, and hybridoma cell lines capable of producing anti-TrkA monoclonal antibodies that block the binding of NGF and TrkA at the cell level were screened by flow cytometry. NGF was biotinylated, and NGF could bind to the extracellular region of TrkA protein on HEK-293T-TrkA cells, and anti-TrkA monoclonal antibodies could also bind to the extracellular region of TrkA protein on HEK-293T-TrkA cells. Competitive experiments were designed to detect the binding of NGF to the extracellular region of TrkA protein on HEK-293T-TrkA cells under the action of anti-TrkA monoclonal antibodies by flow cytometry, and to screen anti-TrkA monoclonal antibodies that block the binding of NGF and TrkA at the cell level. A total of 9 monoclonal hybridoma cell lines capable of producing anti-TrkA monoclonal antibodies that blocked the binding of NGF and TrkA at the cell level were obtained through screening, which are consistent with the results of molecular-level blocking experiments. The experimental results are shown in FIG. 2. In the FIG. 2, The % parent value reflects the NGF signal that binds to the extracellular region of TrkA protein on HEK-293T-TrKA cells. The higher the reading, the stronger the NGF signal that binds to the extracellular region of TrkA protein on HEK-293T-TrKA cells, and the worse the effect of the antibody in blocking the binding of NGF to TrkA; as shown in the FIG. 2, compared with the negative clone group, the % parent value of the 9 positive clone groups is significantly reduced, and the NGF signal binding to the extracellular region of the TrkA protein is significantly reduced; it can be seen that these 9 positive clones can reduce the NGF signal binding to the extracellular region of the TrkA protein on HEK-293T-TrkA cells by generating anti-TrkA monoclonal antibodies that block the binding of NGF to the extracellular region of TrkA protein on HEK-293T-TrkA cells.

### Example 3 Detection of Human-Mouse Cross Reaction of Positive Clones Producing Monoclonal Antibodies by ELISA Method

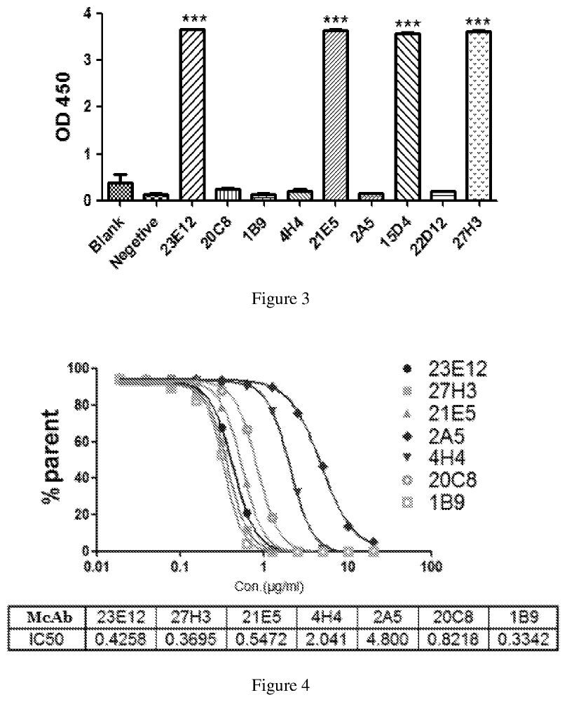

The binding of monoclonal antibodies produced by the supernatant of each positive clone to the Mouse-TrKA receptor was detected by ELISA method, and the human-mouse cross reaction of positive clones producing monoclonal antibodies was detected. The results are shown in FIG. 3. In FIG. 3, the OD₄₅₀ value reflects the strength of the binding of the antibody to the protein. The larger the reading, the stronger the binding of the antibody to the protein. As shown in FIG. 3, the OD₄₅₀ value of the 23E12, 21E5, 15D4, and 27H3 groups is around 3.5, which is significantly different from the negative control group; the OD₄₅₀ value of the 2008, 1B9, 4H4, 3A5, and 22D12 groups were almost close to 0, and there was no significant difference compared with the negative clone group. It can be seen that clones 23E12, 21E5, 15D4, and 27H3 can produce monoclonal antibodies that bind to both Human-TrKA and Mouse-TrKA, and there is a human-mouse cross reaction.

### Example 4 Construction of Vectors

A series of (2008, 23E12, 27H3, 21E5, 2A5, 4H4, 1B9, 22D12, 15D4) chimeric antibody expression vectors were constructed using molecular cloning methods. Chimeric antibodies were recombinantly expressed in the CHO expression system. The nucleotide sequence encoding the light and heavy chain of a series of (2008, 23E12, 27H3, 21E5, 2A5, 4H4, 1B9, 22D12, 15D4) chimeric monoclonal antibodies was obtained through chemical synthesis by Jinweizhi Biotechnology Co., Ltd. The obtained sequence was double-digested and inserted into the same digestion site of the eukaryotic expression vector to construct a series of (2008, 23E12, 27H3, 21E5, 2A5, 4H4, 1B9, 22D12, 15D4) chimeric antibody expression vectors. Then a series of verified correct expression vectors were extracted with Invitrogen plasmid extraction kit, linearized with restriction enzymes, purified and recovered, and stored at −20° C.

### Example 5 Transfection of Vectors Encoding a Series of Chimeric Antibodies and Expression in Cells

After the CHO host cells were resuscitated with CD CHO medium, the cells were collected for transfection when the cell density was about 8*10⁵ cells/mL. The transfected cells were about 1*10⁷ cells, and the vector was about 40 μg. The cells were transfected by electric shock method (Bio-Rad, Gene pulser Xcell). The cells were cultured in 20 mL of CD CHO medium after electric shock. The next day of culture, the cells were collected by centrifugation and resuspended in 20 mL of CD CHO medium with MSX to a final concentration of 50 μM. When the cell density was about 0.6*10⁶ cell/mL, the obtained mixed clone was subcultured with CD CHO medium, and the passage cell density was about 0.2*10⁶ cell/mL. When the cell survival rate was about 90%, the cell culture solution was collected.

### Example 6 Purification of Chimeric Antibodies by Collecting Cell Fermentation Medium

A series of chimeric antibodies were tested at the translation level. Protein A chromatography column was used to purify the collected cell culture solution, and the absorption peaks were collected for mass spectrometry detection. Mass spectrometry detected a series of chimeric antibodies with molecular weights of about 150KD, which was consistent with the theoretical molecular weight. At the same time, the collected samples were detected by 10% SDS-PAGE electrophoresis after reduction and non-reduction. The reduced SDS-PAGE electrophoresis spectrum showed two bands, at about 25KD and 50KD, respectively. The non-reduced SDS-PAGE electrophoresis spectrum showed a single band at about 150KD. The band size of the electrophoresis spectrum is consistent with the theory. The purified sample was dialyzed against 0.02M PBS buffer at pH 7.4 overnight at 4° C.

In the following Examples 7-16, the inventors evaluated the constructed and purified chimeric antibodies 2008, 23E12, 27H3, 21E5, 2A5, 4H4, 1B9, 22D12, 15D4 for their affinity with TrkA, and the activity of binding to TrkA, and blocking NGF to the extracellular region of the TrkA protein.

### Example 7 Evaluation of Blocking Activity of Test Antibodies Using HEK-293T-TrkA Cell Model

NGF was biotinylated, and NGF could bind to the extracellular region of TrkA protein on HEK-293T-TrkA cells, and anti-TrkA monoclonal antibodies could also bind to the extracellular region of TrkA protein on HEK-293T-TrkA cells. A competitive experiment was designed to detect the binding of NGF to the extracellular region of TrkA protein on HEK-293T-TrkA cells under different concentrations (20 μg/mL, 10 μg/mL, 5 μg/mL, 2.5 μg/mL, 1.25 μg/mL, 0.625 μg/mL, 0.313 μg/mL, 0.156 μg/mL, 0.078 μg/mL, 0.039 μg/mL, 0.019 μg/mL) of test antibodies by flow cytometry, and to study the inhibitory effect of test antibodies on the binding of NGF and TrKA. The experimental results are shown in FIG. 4. In the FIG. 4, the parent % value reflects the NGF signal binding to the extracellular region of TrKA protein on HEK-293T-TrkA cells. The lower the reading, the weaker the NGF signal that binds to the extracellular region of the TrkA protein on HEK-293T-TrkA cells, and the greater the effect of the antibody on inhibiting the binding of NGF to TrKA; as shown in FIG. 4, as the test antibody concentration increases, the parent % value gradually decreases until it approaches zero, i.e., the NGF signal binding to the extracellular region of TrkA protein gradually decreases until there is no NGF binding to the extracellular region of the TrkA protein, and the binding of NGF to TrkA is all inhibited. It can be seen that within a certain concentration range, each test antibody can dose-dependently inhibit the binding of NGF to TrkA at the cellular level.

### Example 8 Evaluation of the Activity of Test Antibodies In Vitro Using the NIH-3T3-TrkA Cell Model

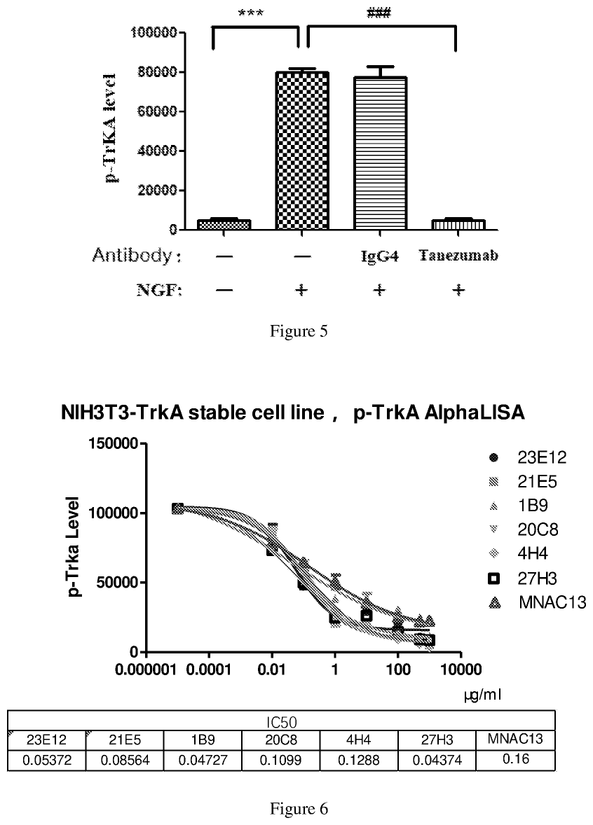

Under NGF stimulation, tyrosine phosphorylation level of the TrkA protein on the membrane of NIH-3T3-TrkA cells was up-regulated, and the TrkA downstream signaling pathway was activated. The test antibody could bind to the TrkA protein on the surface of NIH-3T3-TrkA cell membrane, inhibit NGF stimulation, and down-regulate tyrosine phosphorylation level of TrkA protein. In the test, IgG4 was used as a negative control (the antibody does not bind to NGF and TrkA), tanezumab (anti-NGF monoclonal antibody, the amino acid sequence of the light chain is shown in SEQ ID NO: 149, and the amino acid sequence of the heavy chain is shown in SEQ ID NO: 150, a drug that inhibits the NGF-TrkA pathway) and MNAC13 (anti-TrKA monoclonal antibody, the amino acid sequence of the light chain is shown in SEQ ID NO: 151, the amino acid sequence of the heavy chain is shown in SEQ ID NO: 152, a drug that inhibits the NGF-TrkA pathway) were used as a positive control. Among them, Tanezumab was used to verify whether the NIH-3T3-TrkA cell model could be used to evaluate the in vitro activity of drugs that inhibited the NGF-TrkA pathway (test drugs). The experimental data are shown in FIG. 5. The method of AlphaLISA was used to detect the down-regulation of tyrosine phosphorylation of TrkA protein under the action of different concentrations of test antibody and positive antibody MNAC13, and to measure the activity of test antibodies in vitro. The test results of p-TrkA are shown in FIG. 6. The experimental results show that both the test antibody and the positive antibody MNAC13 can inhibit the NGF-TrKA pathway and dose-dependently down-regulate the tyrosine phosphorylation level of TrkA protein. Among them, the half inhibitory concentration IC₅₀ of the test antibodies 23E12, 2008, 27H3, 21E5, 1B9, and 4H4 is smaller than of the positive antibody MNAC13. It can be seen that the test antibodies 23E12, 2008, 27H3, 21E5, 1B9, 4H4 have better activity in vitro than the positive antibody MNAC13.

(SEQ ID NO: 149)
DIQMTQSPSSLSASVGDRVTITCRASQSISNNLNWYQQKPGKAPKLLIYY
TSRFHSGVPSRFSGSGSGTDFTFTISSLQPEDIATYYCQQEHTLPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.
(SEQ ID NO: 150)
QVQLQESGPGLVKPSETLSLTCTVSGFSLIGYDLNWIRQPPGKGLEWIGI
IWGDGTTDYNSAVKSRVTISKDTSKNQFSLKLSSVTAADTAVYYCARGGY
WYATSYYFDYWGQGTLVTVSSASTKGPSVFPLAPCSRSTSESTAALGCLV
KDYFPEPVTVSWNSGALTSGVHTFPAVLQSSGLYSLSSVVTVPSSNFGTQ
TYTCNVDHKPSNTKVDKTVERKCCVECPPCPAPPVAGPSVFLFPPKPKDT
LMISRTPEVTCVVVDVSHEDPEVQFNWYVDGVEVHNAKTKPREEQFNSTF
RVVSVLTVVHQDWLNGKEYKCKVSNKGLPAPIEKTISKTKGQPREPQVYT
LPPSREEMTKNQVSLTCLVKGFYPSDIAVEWESNGQPENNYKTTPPMLDS
DGSFFLYSKLTVDKSRWQQGNVFSCSVMHEALHNHYTQKSLSLSPGK.
(SEQ ID NO: 151)
DIVLTQSPSSLSASVGDRVTITCSASSSVSYMHWYQQKPGQAPKLLIYTT
SNLASGVPSRFSGSGSGTDYTLTISSLQPEDVATYYCHQWSSYPWTFGGG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.
(SEQ ID NO: 152)
EVQLLESGGGLVQPGGSLRLSCAASGFTFSTYTMSWARQAPGKGLEWVAY
ISKGGGSTYYPDTVKGRFTISRDNSKNTLYLQMNSLRAEDSAVYYCARGA
MFGNDFFFPMDRWGQGTLVTVSSASTKGPSVFPLAPCSRSTSESTAALGC
LVKDYFPEPVTVSWNSGALTSGVHTFPAVLQSSGLYSLSSVVTVPSSSLG
TKTYTCNVDHKPSNTKVDKRVESKYGPPCPPCPAPEAAGGPSVFLFPPKP
KDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPV
LDSDGSFFLYSKLTVDKSRWQEGNVFSCSVMHEALHNHYTQKSLSLSLG.

### Example 9 Evaluation of the Affinity of Test Antibodies to TrkA Using the Fortebio Method

The test antibody (23E12, 2008, 21E5, 27H3, 1B9, 4H4, 2A5) samples were diluted with buffer solution (PBS buffer 100 ml, 0.1 gBSA was added and stirred until fully dissolved, then 20 μL Tween 20 was added, well mixed) to 4 concentration gradients (80 nM, 26.67 nM, 8.89 nM, 2.93 nM), respectively. They were added to each well of the sample plate. Through the affinity detection system (OCTET RED 96 SYSTEM), after the TRKA (50 μg/ml, 75 kDa, provided by HEC Labs) protein with a histidine tag bound to a Ni-NTA sensor (Manufacturer: PALL, article NO: 18-5101), the binding of the TRKA protein with each test antibody was automatically detected. The results are shown in Table 1. The K_{D} value is the equilibrium dissociation constant between the antibody and its antigen, and the K_{D} value is inversely proportional to the affinity. The K_{D} value is related to the antibody concentration (the amount of antibody required for a particular experiment). The lower the K_{D} value (the lower the concentration), the higher the affinity of the antibody. High-affinity antibodies are generally considered to be in the low nanomolar range (10⁻⁹). The results in Table 1 show that the K_{D} values of the test antibodies (23E12, 2008, 21E5, 27H3, 1B9, 4H4, 2A5) are in the low nanomolar range (10⁻⁹), indicating that the test antibodies have high affinity.

TABLE 1

| Sample ID | K_{D} (M) | kon(1/Ms) | kon Error | kdis(1/s) | kdis Error | Full R\hat{ }2 |
|---|---|---|---|---|---|---|
| 23E12 | 5.70E−11 | 1.90E+06 | 6.33E+04 | 1.09E−04 | 7.47E−06 | 0.944 |
| 4H4 | 1.68E−09 | 1.39E+05 | 6.01E+03 | 2.34E−04 | 1.55E−05 | 0.961 |
| 20C8 | 1.15E−10 | 5.51E+05 | 2.34E+04 | 6.33E−05 | 1.17E−05 | 0.949 |
| 27H3 | 1.78E−10 | 1.57E+06 | 4.53E+04 | 2.79E−04 | 7.91E−06 | 0.929 |
| 21E5 | 1.73E−10 | 1.01E+06 | 2.08E+04 | 1.75E−04 | 5.60E−06 | 0.977 |
| 1B9 | 8.99E−10 | 1.01E+06 | 6.65E+04 | 9.04E−04 | 6.29E−05 | 0.910 |
| 2A5 | 1.62E−11 | 2.22E+05 | 1.42E+04 | 3.60E−06 | 1.96E−05 | 0.944 |

### Example 10 Evaluation of Affinity of Test Antibodies by Flow Cytometry

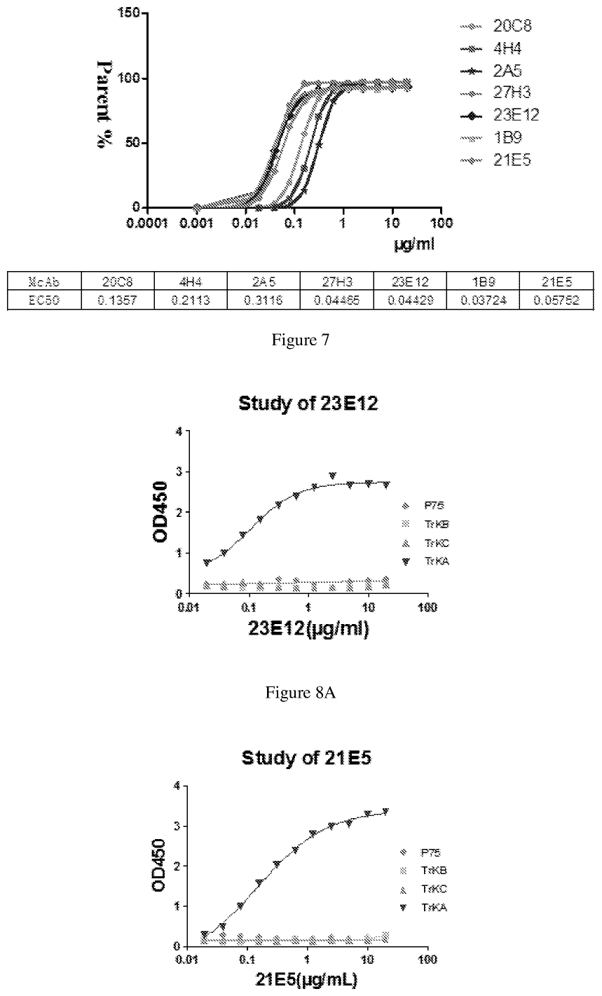

The test antibody (23E12, 2008, 21E5, 27H3, 1B9, 4H4, 2A5) samples were diluted with PBS buffer solution to 11 concentration gradients (20 μg/mL, 10 μg/mL, 5 μg/mL, 2.5 μg/mL, 1.25 μg/mL, 0.625 μg/mL, 0.313 μg/mL, 0.156 μg/mL, 0.078 μg/mL, 0.039 μg/mL, 0.019 μg/mL). Flow cytometry was used to detect the binding of test antibodies at various concentration gradients to the TrKA receptors on the surface of HEK-293T-TrKA cells, and the affinity of the test antibodies was evaluated at the cell level. The results are shown in FIG. 7. In FIG. 7, the EC₅₀ (half-binding concentration) value reflects the affinity of the antibody. The smaller the EC₅₀ value, the higher the affinity of the antibody. It is generally considered that the EC₅₀ value of the high-affinity antibody is less than 1.5 μg/mL. The results in FIG. 7 show that the EC₅₀ values of the test antibodies 23E12, 2008, 21E5, 27H3, 1B9, 4H4, and 2A5 are all lower than 1.5 μg/mL, indicating that the test antibodies have high affinity.

### Example 11 Evaluation of Specificity of Binding of Test Antibodies to Target TrKA by ELISA Method

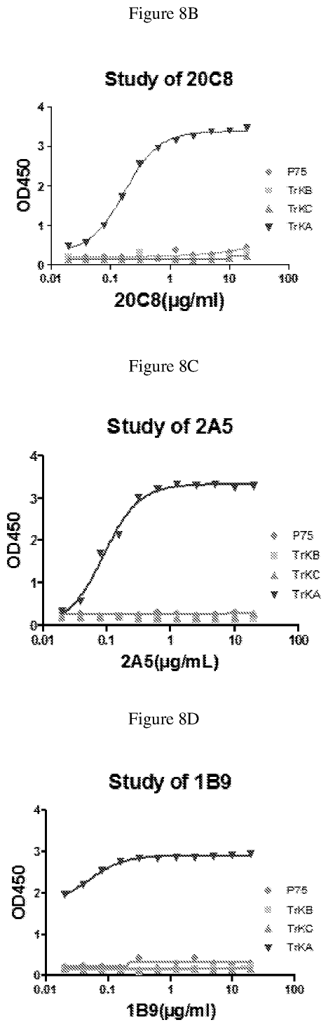

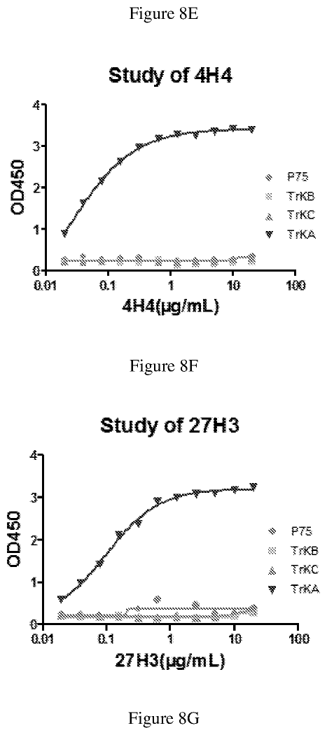

The TrkA receptor family belongs to receptor tyrosine kinases (RTKs), including TrkA, TrkB, and TrkC, which have high homology. TrkA is a receptor tyrosine kinase of nerve growth factor (NGF) that selectively binds to NGF and is a functional receptor for NGF. In addition to the high-affinity receptor TrkA, NGF can also bind to its low-affinity receptor p75. In the test, the binding of test antibodies at different concentrations (20 μg/mL, 10 μg/mL, 5 μg/mL, 2.5 μg/mL, 1.25 μg/mL, 0.625 μg/mL, 0.313 μg/mL, 0.156 μg/mL, 0.078 μg/mL, 0.039 μg/mL, 0.019 μg/mL) to TrKA, TrKB, TrKC, and P75 respectively was detected by the ELISA method, and the specificity of the test antibody binding to the target TrKA was evaluated. The results are shown in FIG. 8. In the FIG. 8, at a certain antibody concentration, the OD₄₅₀ value reflects the binding strength of the antibody to the protein. The larger the reading, the stronger the binding of the antibody to the protein. The experimental results shows that the tested antibodies 23E12, 2008, 27H3, 21E5, 1B9, 4H4, and 2A5 all have good binding to the TrKA receptor (The concentration of the test antibody increases from 0 μg/mL to 20 μg/mL, and the OD₄₅₀ value gradually increases until it becomes stable, which is close to about 3), but did not bind to TrKB, TrKC, P75(The concentration of each test antibody increases from 0 μg/mL to 20 μg/mL, and the OD₄₅₀ value remains almost unchanged, which is close to 0.). It can be seen that the specificity of the binding of the test antibodies to the target TrKA is very good.

### Example 12 Detection of Binding Ability of Test Antibodies to Mouse-TrKA Protein by ELISA Method

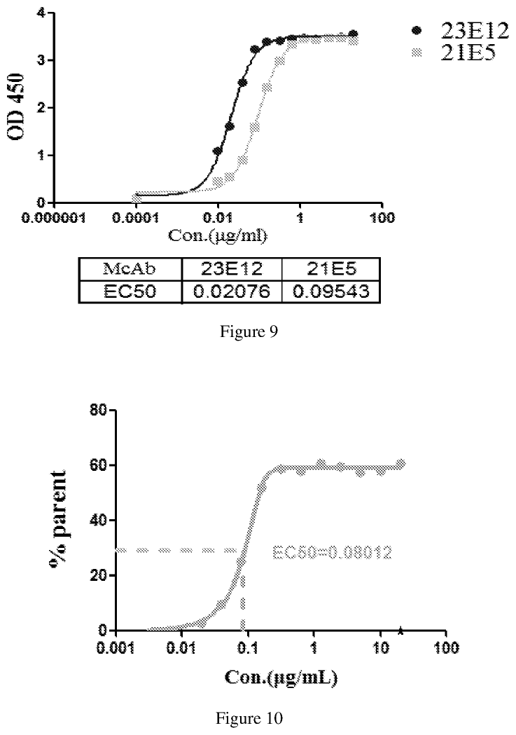

Test antibody (23E12, 21E5) samples were diluted with PBS buffer solution to 11 concentration gradients (20 μg/mL, 10 μg/mL, 5 μg/mL, 2.5 μg/mL, 1.25 μg/mL, 0.625 g/mL, 0.313 μg/mL, 0.156 μg/mL, 0.078 μg/mL, 0.039 μg/mL, 0.019 μg/mL). The binding of the test antibody to the Mouse-TrKA receptor at each concentration gradient was detected by the ELISA method, thus the binding ability of the test antibody to the Mouse-TrKA protein was detected. The results are shown in FIG. 9. In FIG. 9, at a certain antibody concentration, the OD₄₅₀ value reflects the binding strength of the antibody to the protein. The larger the reading, the stronger the binding of the antibody to the protein. The experimental results show that the concentration of the test antibody increases from 0 μg/mL to 20 μg/mL, and the OD₄₅₀ value gradually increases until it approaches stability, which is close to about 3.5. It can be seen that the test antibodies 23E12, 21E5, and Mouse-TrKA protein all have good binding ability.

### Example 13 Detection of Binding Ability of the Test Antibody to Mouse-TrKA Protein by Flow Cytometry

The test antibody 23E12 samples were diluted with PBS buffer to 11 concentration gradients (20 μg/mL, 10 μg/mL, 5 μg/mL, 2.5 μg/mL, 1.25 μg/mL, 0.625 μg/mL, 0.313 μg/mL, 0.156 μg/mL, 0.078 μg/mL, 0.039 μg/mL, 0.019 μg/mL), and the binding of the test antibody to the Mouse-TrKA receptor on the surface of HEK293T-Mouse-TrKA cells at each concentration gradient was detected by flow cytometry. The binding ability of the antibody to Mouse-TrKA protein was tested. The results are shown in FIG. 10. In FIG. 10, the EC₅₀ (half the binding concentration) value reflects the binding capacity of the antibody. The smaller the EC₅₀ value, the stronger the binding capacity of the antibody. The experimental results show that the concentration of the test antibody increases from 0 μg/mL to 20 μg/mL, the % Parent value gradually increases until it approaches stability, and the EC₅₀ value=0.08012 μg/mL. It can be seen that the test antibody 23E12 has a good binding ability with the Mouse-TrKA receptor on the surface of HEK293T-Mouse-TrKA cells.

### Example 14 Detection of Inhibitory Effect of Test Antibodies on the Binding of Mouse-NGF and Mouse-TrKA by ELISA Method

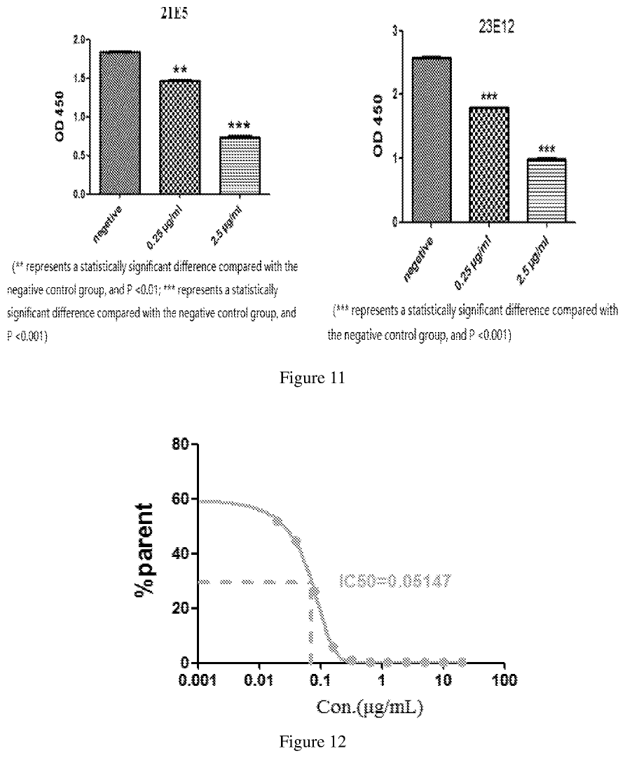

In the test, IgG4 was used as a negative control (the antibody does not bind to Mouse-NGF and Mouse-TrkA), and Mouse-NGF was biotinylated. Mouse-NGF could bind to Mouse-TrkA protein, and anti-Mouse-TrkA monoclonal antibody could also bind to Mouse-TrkA protein. A competitive experiment was designed to detect the binding of Mouse-NGF and Mouse-TrkA protein under different concentrations (2.5 μg/mL, 0.25 μg/mL) of test antibodies (23E12, 21E5) by ELISA method, Thus the inhibition effect of test antibodies on the binding of Mouse-NGF and Mouse-TrKA was investigated. The experimental results are shown in FIG. 11. In FIG. 11, the OD₄₅₀ value reflects the Mouse-NGF signal binding to Mouse-TrkA. The lower the reading, the weaker the Mouse-NGF signal that binds to Mouse-TrkA, and the greater the effect of the antibody on inhibiting the binding of Mouse-NGF and Mouse-TrKA; as shown in FIG. 11, compared with the negative control group, the mouse-NGF signal binding to Mouse-TrkA was significantly reduced under the effects of test antibodies (23E12, 21E5) at different concentrations (2.5 μg/mL, 0.25 μg/mL). It can be seen that the test antibodies 23E12 and 21E5 can inhibit the binding of Mouse-NGF and Mouse-TrKA.

### Example 15 Detection of the Inhibitory Effect of the Test Antibody on the Binding of Mouse-NGF and Mouse-TrKA by Flow Cytometry

Mouse-NGF was biotinylated. Mouse-NGF could bind to the extracellular region of Mouse-TrkA protein on HEK293 T-Mouse-TrkA cells, and anti-Mouse-TrkA monoclonal antibody could also bind to the extracellular region of Mouse-TrkA protein on HEK293T-Mouse-TrkA cells. A competitive experiment was designed to detect the binding of Mouse-NGF to the extracellular region of Mouse-TrkA protein on HEK293T-Mouse-TrkA cells under different concentrations (20 μg/mL, 10 μg/mL, 5 μg/mL, 2.5 μg/mL, 1.25 μg/mL, 0.625 μg/mL, 0.313 μg/mL, 0.156 μg/mL, 0.078 μg/mL, 0.039 μg/mL, 0.019 μg/mL) of test antibodies by flow cytometry, thus the inhibition effect of test antibodies on the binding of Mouse-NGF and Mouse-TrKA was investigated. The experimental results are shown in FIG. 12. In FIG. 12, the parent % value reflects the Mouse-NGF signal that binds to the extracellular region of the Mouse-TrKA protein on the HEK293T-Mouse-TrkA cell. The lower the reading, the weaker the Mouse-NGF signal that binds to the extracellular region of the Mouse-TrkA protein on the HEK293T-Mouse-TrkA cell, and the greater the effect of the antibody on inhibiting the binding of Mouse-NGF and Mouse-TrKA; as shown in FIG. 12, as the concentration of the test antibody increases, the % parent value gradually decreases until it approaches zero, that is, the signal of Mouse-NGF binding to the extracellular region of Mouse-TrkA protein gradually decreases until no Mouse-NGF binds to the extracellular region of Mouse-TrkA protein, and the binding of Mouse-NGF and Mouse-TrkA is all inhibited, IC50=0.05147 μg/mL. It can be seen that within a certain concentration range, the test antibody 23E12 can dose-dependently inhibit the binding of Mouse-NGF and Mouse-TrkA at the cellular level.

### Example 16 Detection of the Inhibitory Effect of the Test Antibody on the Binding of Human-NGF and Human-TrKA by ELISA Method

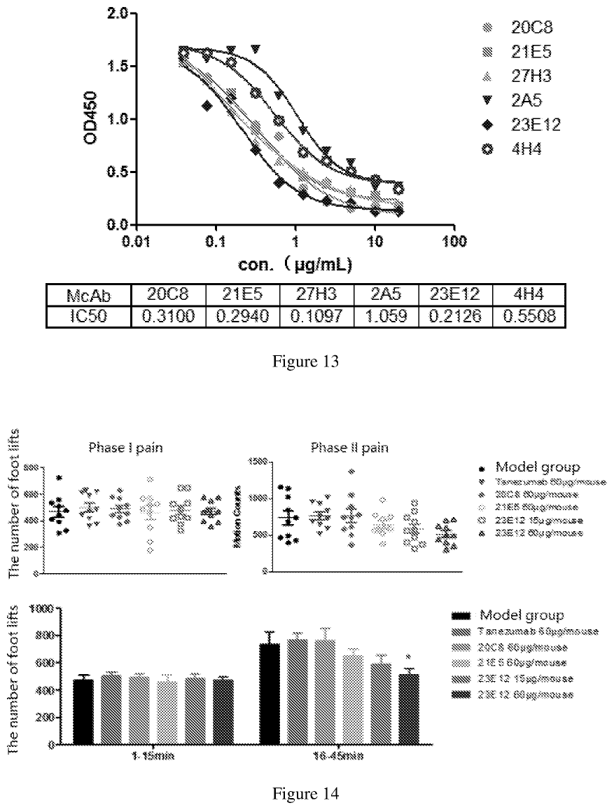

Mouse-NGF was biotinylated. Mouse-NGF could bind to Mouse-TrkA protein, and anti-Mouse-TrkA monoclonal antibody could also bind to Mouse-TrkA protein. A competitive experiment was designed to detect the binding of Mouse-NGF and Mouse-TrkA protein under different concentrations (20 μg/mL, 10 μg/mL, 5 μg/mL, 2.5 μg/mL, 1.25 μg/mL, 0.625 μg/mL, 0.313 μg/mL, 0.156 μg/mL, 0.078 μg/mL, 0.039 μg/mL, 0.019 μg/mL) of test antibodies by ELISA method, thus the inhibition effect of test antibodies on the binding of Mouse-NGF and Mouse-TrkA was investigated. The experimental results are shown in FIG. 13. In FIG. 13, the OD₄₅₀ value reflects the Mouse-NGF signal binding to Mouse-TrkA. The lower the reading, the weaker the Mouse-NGF signal binding to Mouse-TrkA, and the greater the effect of the antibody on inhibiting the binding of Mouse-NGF and Mouse-TrKA; as shown in FIG. 13, as the concentration of the test antibody increases, the OD₄₅₀ value gradually decreases until it approaches zero, that is, the signal of Mouse-NGF binding to the extracellular region of Mouse-TrkA protein gradually decreases until almost no Mouse-NGF binds to the extracellular region of Mouse-TrkA protein, and the binding of Mouse-NGF and Mouse-TrkA is almost completely inhibited. It can be seen that within a certain concentration range, the test antibodies 23E12, 2008, 21E5, 27H3, 4H4, 2A5 can dose-dependently inhibit the binding of NGF and TrkA at the molecular level.

### Example 17 Evaluation of In Vivo Analgesic Activity of Test Antibodies Using Formalin-Induced Pain Model

The formalin inflammatory pain model is a validated pain model that produces continuous rather than transient pain stimuli and responses by injecting formalin. This model produces two-phase pain, which are phase I chemically stimulated pain and phase II inflammatory pain. The pain response caused by this model is reproducible and measurable. This model is one of the best models for preclinical pain research, and is widely used to evaluate the analgesic effect of different drugs. In the test, male ICR mice of 6-8 weeks were selected and randomly divided into 6 groups according to body weight before modeling: model group (subcutaneous injection of normal saline), Tanezumab 60 μg/mouse dose group, 20C8 60 μg/mouse dose group, 21E5 60 μg/mouse dose group, 23E12 15 μg/mouse dose group, 23E12 60 μg/mouse dose group, 10 mice in each group. The drug was administered subcutaneously. After 18 hours, 15 μL of 2.5% formalin solution was injected subcutaneously into the back of the right hind paw of the mouse. The number of times that the mouse lifted the foot within 45 minutes was observed. 1-15 min is Phase I pain and 16-45 min is Phase II pain. Generally speaking, Phase I pain reflects acute pain, and Phase II pain reflects chronic pain. The results are shown in FIG. 14 (the data in FIG. 14 are Mean±SEM, n=10/group, * p<0.05 compared with the solvent model group, using single factor analysis of variance plus LSD multiple comparison test). The number of foot lifts in FIG. 14 reflects the pain intensity of the mouse after modeling. The lower the number of foot lifts, the weaker the pain. The results show that compared with the model group, the 23E12 60 μg/mouse dose group can significantly reduce the number of foot lifts in phase II pain of the mouse, and the other dose groups fail to reduce the number of foot lifts of the mouse after modeling. Conclusion: in the formalin-induced pain model, subcutaneous injection of the test antibody 23E12 at 60 μg/mouse is effective for chronic pain, while the positive drug Tanezumab does not exhibit analgesic effects and may not be sensitive to the formalin-induced pain model.

### Example 18 Evaluation of In Vivo Analgesic Activity of Test Antibodies Using a Complete Freund's Adjuvant-Induced Inflammation Pain Model

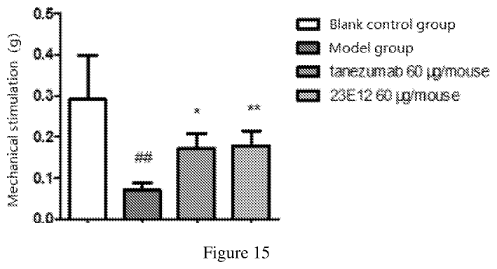

Complete Freund's adjuvant-induced inflammatory pain model is a pain model that produces chronic inflammatory pain stimuli similar to osteoarthritis and responses by injecting complete Freund's adjuvant in the palms of mice. The pain is measured by the mechanical pain test. The greater the intensity of the mechanical stimulus, the more resistant the animal is to pain. In the test, 8-week-old male ICR mice were selected and randomly divided into 6 groups (15 numbers/group) according to pain sensitivity before modeling: control group (injection of saline 25 μl+subcutaneous injection of saline in the left hind limb plantar of mice), model group (injection of 25 μl CFA induced inflammatory pain+subcutaneous injection of saline in the left hind limb of mice), Tanezumab 60 μg/mouse dose group, 23E12 60 μg/mouse dose group, the drug was injected subcutaneously on the 4th day after modeling, and a mechanical pain test was performed after 36 hr of administration. The results are shown in FIG. 15 (the data in FIG. 15 are Mean±SEM, n=15/group, #p □ 0.05, ## p □ 0.01 compared with the blank control group, * p □ 0.05, ** p □ 0.01 compared with the model group, using nonparametric statistical analysis plus independent sample T test). The ordinate represents the intensity of mechanical stimulus. The greater the pressure threshold of mouse paw withdrawal, the better the analgesic effect. The results show that compared with the model group, subcutaneous injection of 60 μg/mouse 23E12 and 60 μg/mouse tanezumab can significantly increase the pressure threshold of mouse paw withdrawal (P<0.01; P<0.05) and exhibit analgesic effects. Both analgesic effects are equivalent. Conclusion: subcutaneous injection of the test antibody 23E12 at 60 μg/mouse exhibits analgesic effects in a complete Freund's adjuvant-induced inflammatory pain model.

### Example 19: Toxicology Test of Test Antibodies

In this example, the toxicity test to be performed by the inventors is shown in Table 2 below.

TABLE 2

| | Mode | Species or | | |
|---|---|---|---|---|
| Research | of admin- | inquiry | | |
| project | istration | system | Main research | Results |
| Toxicity | i.v. | SD rat | Observing whether | No animal died, and diffuse |
| of single | | cynomolgus | target organ toxicity | inflammatory infiltration was |
| administration | | monkey | occurs. Observing the | observed on organs and tissues, but |
| | | | related symptoms, the | no target organ toxicity was shown |
| | | | impact on body | regardless of clinical pathology or |
| | | | weight, food intake, | histological analysis. It had no |
| | | | ophthalmoscope | significant effect on body weight, |
| | | | examination, | food intake, ophthalmoscope |
| | | | electrocardiogram, | examination, electrocardiogram, |
| | | | hematology, clinical | hematology, clinical biochemistry, |
| | | | biochemistry, urine, | urine, organ weight. There was no |
| | | | organ weight; the | significant abnormality in the gross |
| | | | maximum tolerated | anatomy. |
| | | | dose MTD, and | |
| | | | sufficient safety factor | |
| | | | were provided, | |
| Toxicity | i.v./ | SD rat | Observing clinical | No animal died, and diffuse |
| of | (Once a | cynomolgus | pathology and | inflammatory infiltration was |
| repeated | week, 4 | monkey | histology to determine | observed on organs and tissues, but |
| administration | weeks) | | whether there is target | no target organ toxicity was shown |
| | | | organ toxicity | regardless of clinical pathology or |
| | | | NOAEL, observing | histological analysis. There were no |
| | | | the effects on body | symptoms related to nivolumab, |
| | | | weight, food intake, | and it had no significant effect on |
| | | | ophthalmoscope | body weight, food intake, |
| | | | examination, | ophthalmoscope examination, |
| | | | electrocardiogram, | electrocardiogram, hematology, |
| | | | hematology, clinical | clinical biochemistry, urine, and |
| | | | biochemistry, urine, | organ weight. There was no |
| | | | organ weight; safty | significant abnormality in the gross |
| | | | window was | anatomy. |
| | | | calculated by | |
| | | | immunogenicity | |
| | | | (ADA (anti-drug | |
| | | | antibody) analysis | |
| | | | method), | |
| | | | immunotoxicity | |
| | | | studies (evaluation | |
| | | | includes: hematology | |
| | | | differential count of | |
| | | | white blood cells | |
| | | | (including | |
| | | | macrophages), clinical | |
| | | | chemistry (globulin | |
| | | | and albumin: globulin | |
| | | | ratio)), organ mass | |
| | | | (thymus, spleen, | |
| | | | lymph nodes) and | |
| | | | histopathology | |
| | | | (lymphatic organs and | |
| | | | tissues) | |
| General | i.v./ | Central | The main research | The drug has no effect on the |
| Safety | Single | nervous | focus is on the | central nervous system, |
| Pharmacology | dose | system | potential undesired | cardiovascular system and |
| | | Respiratory | adverse effects on | respiratory system. |
| | | system | physiological | |
| | | Cardiovascular | functions when the | |
| | | system | dosage of the drug is | |
| | | | within or above the | |
| | | | therapeutic range, that | |
| | | | is, to observe the | |
| | | | effects of the drug on | |
| | | | the central nervous | |
| | | | system, cardiovascular | |
| | | | system and respiratory | |
| | | | system. | |
| Local | Vascular | Rabbit | Testing whether drugs | No effect |
| drug | irritation | | have effects on blood | |
| safety test | test | | vessels and blood | |
| | and in | | | |
| | vitro | | | |
| | hemolysis | | | |
| | test | | | |
| High | Guinea | Guinea pig | Determining whether | No sensitization |
| sensitization | pig | | there is sensitization | |
| test | whole | | | |
| | body | | | |
| | active | | | |
| | allergy | | | |
| | test | | | |

Among them, the above experimental process and experimental conclusions are as follows:

toxicology experiment:

toxicity of single administration

SD rats and cynomolgus monkeys were observed for target organ toxicity. The related symptoms, such as the impact on body weight, food intake, ophthalmoscope examination, electrocardiogram, hematology, clinical biochemistry, urine and organ weight were observed. The maximum tolerated dose MTD and sufficient safety factor were provided. The results showed that there were no animal deaths, and diffuse inflammatory infiltration was observed on organs and tissues, but no target organ toxicity was shown in clinical pathology or histological analysis. It had no significant effect on body weight, food intake, ophthalmoscope examination, electrocardiogram, hematology, clinical biochemistry, urine and organ weight. There was no significant abnormality in the gross anatomy.

Toxicity of repeated administration: SD rats and cynomolgus monkeys were injected with the drug intravenously once a week for 4 consecutive weeks. The clinical pathology and histology were observed to determine whether there was target organ toxicity NOAEL. The impact on weight, food intake, ophthalmoscope examination, electrocardiogram, hematology, clinical biochemistry, urine and organ weight were observed. Safety window was calculated by immunogenicity (ADA (anti-drug antibody) analysis method), immunotoxicity studies (evaluation includes: hematology differential count of white blood cells (including macrophages), clinical chemistry (globulin and albumin: globulin ratio)), organ mass (thymus, spleen, lymph nodes) and histopathology (lymphatic organs and tissues). The results showed that no animals died, and diffuse inflammatory infiltration was observed on organs and tissues, but no target organ toxicity was shown regardless of clinical pathology or histological analysis. There were no symptoms related to nivolumab, and it had no significant effect on body weight, food intake, ophthalmoscope examination, electrocardiogram, hematology, clinical biochemistry, urine, and organ weight. There was no significant abnormality in the gross anatomy.

General safety pharmacology: SD rats were injected with the drug intravenously in single administration. The effects of the drug on the central nervous system, cardiovascular system and respiratory system were observed. The results showed that the drug had no effect on the central nervous system, cardiovascular system and respiratory system.

Local administration safety test (vascular irritation test and in vitro hemolysis test): New Zealand white rabbits were injected with the drug intravenously to test whether the drug had effects on blood vessels and blood. The results showed that the drug had no effect on blood vessels and blood.

High sensitization test (whole body active allergy test in guinea pigs): guinea pigs were injected with the drug intravenously in single administration to observe whether the drug had sensitization. The results showed that the drug had no sensitization.

Reference throughout this specification to “an embodiment”, “some embodiments”, “one embodiment”, “another example”, “an example”, “a specific example” or “some examples” means that a particular feature, structure, material, or characteristic described in connection with the embodiment or example is included in at least one embodiment or example of the present disclosure. Thus, the appearances of the phrases such as “in some embodiments”, “in one embodiment”, “in an embodiment”, “in another example”, “in an example”, “in a specific example” or “in some examples” in various places throughout this specification are not necessarily referring to the same embodiment or example of the present disclosure. Furthermore, the particular features, structures, materials, or characteristics may be combined in any suitable manner in one or more embodiments or examples. In addition, without any contradiction, those skilled in the art may combine different embodiments or examples and features of the different embodiments or examples described in this specification.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

1.-59. (canceled)

60. An antibody or antigen-binding fragment thereof capable of specifically recognizing TrkA, wherein the antibody comprises a CDR sequence selected from at least one of the following:
heavy chain variable region CDR1, CDR2, CDR3 sequences as shown in SEQ ID NO: 1, 2 and 3, or an amino acid sequence having at least 95% identity with SEQ ID NO: 1, 2 and 3, respectively; or
heavy chain variable region CDR1, CDR2, CDR3 sequences as shown in SEQ ID NO: 4, 5 and 6, or an amino acid sequence having at least 95% identity with SEQ ID NO: 4, 5 and 6, respectively; or
heavy chain variable region CDR1, CDR2, CDR3 sequences as shown in SEQ ID NO: 7, 8 and 9, or an amino acid sequence having at least 95% identity with SEQ ID NO: 7, 8 and 9, respectively; or
heavy chain variable region CDR1, CDR2, CDR3 sequences as shown in SEQ ID NO: 10, 11 and 12, or an amino acid sequence having at least 95% identity with SEQ ID NO: 10, 11 and 12, respectively; or
heavy chain variable region CDR1, CDR2, CDR3 sequences as shown in SEQ ID NO: 13, 14 and 15, or an amino acid sequence having at least 95% identity with SEQ ID NO: 13, 14 and 15, respectively; or
heavy chain variable region CDR1, CDR2, CDR3 sequences as shown in SEQ ID NO: 16, 17 and 18, or an amino acid sequence having at least 95% identity with SEQ ID NO: 16, 17 and 18, respectively; or
heavy chain variable region CDR1, CDR2, CDR3 sequences as shown in SEQ ID NO: 19, 20 and 21, or an amino acid sequence having at least 95% identity with SEQ ID NO: 19, 20 and 21, respectively; or
heavy chain variable region CDR1, CDR2, CDR3 sequences as shown in SEQ ID NO: 22, 23 and 24, or an amino acid sequence having at least 95% identity with SEQ ID NO: 22, 23 and 24, respectively; or
heavy chain variable region CDR1, CDR2, CDR3 sequences as shown in SEQ ID NO: 25, 26 and 27, or an amino acid sequence having at least 95% identity with SEQ ID NO: 25, 26 and 27, respectively; and
light chain variable region CDR1, CDR2, CDR3 sequences as shown in SEQ ID NO: 28, 29 and 30, or an amino acid sequence having at least 95% identity with SEQ ID NO: 28, 29 and 30, respectively; or
light chain variable region CDR1, CDR2, CDR3 sequences as shown in SEQ ID NO: 31, 32 and 33, or an amino acid sequence having at least 95% identity with SEQ ID NO: 31, 32 and 33, respectively; or
light chain variable region CDR1, CDR2, CDR3 sequences as shown in SEQ ID NO: 34, 35 and 36, or an amino acid sequence having at least 95% identity with SEQ ID NO: 34, 35 and 36, respectively; or
light chain variable region CDR1, CDR2, CDR3 sequences as shown in SEQ ID NO: 37, 38 and 39, or an amino acid sequence having at least 95% identity with SEQ ID NO: 37, 38 and 39, respectively; or
light chain variable region CDR1, CDR2, CDR3 sequences as shown in SEQ ID NO: 40, 41 and 42, or an amino acid sequence having at least 95% identity with SEQ ID NO: 40, 41 and 42, respectively; or
light chain variable region CDR1, CDR2, CDR3 sequences as shown in SEQ ID NO: 43, 44 and 45, or an amino acid sequence having at least 95% identity with SEQ ID NO: 43, 44 and 45, respectively; or
light chain variable region CDR1, CDR2, CDR3 sequences as shown in SEQ ID NO: 46, 47 and 48, or an amino acid sequence having at least 95% identity with SEQ ID NO: 46, 47 and 48, respectively; or
light chain variable region CDR1, CDR2, CDR3 sequences as shown in SEQ ID NO: 49, 50 and 51, or an amino acid sequence having at least 95% identity with SEQ ID NO: 49, 50 and 51, respectively; or
light chain variable region CDR1, CDR2, CDR3 sequences as shown in SEQ ID NO: 52, 53 and 54, or an amino acid sequence having at least 95% identity with SEQ ID NO: 52, 53 and 54, respectively.

61. The antibody or antigen-binding fragment thereof according to claim 60, wherein the antibody or antigen-binding fragment thereof specifically recognizes the extracellular region of TrkA.

62. The antibody or antigen-binding fragment thereof according to claim 60, wherein the antibody has a heavy chain variable region of the amino acid sequence shown in any one of SEQ ID NO: 55˜63.

63. The antibody or antigen-binding fragment thereof according to claim 60, wherein the antibody has a light chain variable region of the amino acid sequence shown in any one of SEQ ID NO: 64˜72.

64. The antibody or antigen-binding fragment thereof according to claim 60, wherein the full-length sequence of the constant region of the antibody is shown in SEQ ID NO: 74 or 75.

65. The antibody or the antigen-binding fragment thereof according to claim 60, wherein the antibody has a heavy chain of the amino acid sequence shown in any one of SEQ ID NO: 76˜84 and a light chain of the amino acid sequence shown in any one of SEQ ID NO: 85˜93.

66. The antibody or antigen-binding fragment thereof according to claim 60, wherein the antibody is a single chain antibody fragment, a multimeric antibody, a CDR-grafted antibody, or a small molecule antibody;
wherein the antibody is a single chain antibody fragment, and the antibody has the amino acid sequence shown in SEQ ID NO: 94˜111.

67. A nucleic acid molecule, wherein the nucleic acid molecule encodes the antibody or the antigen-binding fragment thereof according to claim 60;
wherein the nucleic acid molecule is DNA;
wherein the nucleic acid molecule has a nucleotide sequence as shown in any one of SEQ ID NO: 112˜120, or a nucleotide sequence as shown in any one of SEQ ID NO: 121˜129, or a nucleotide sequence as shown in any one of SEQ ID NO: 130˜147.

68. A pharmaceutical composition comprising the antibody or antigen-binding fragment thereof according to claim 60.

69. A kit for detecting TrkA, comprising the antibody or antigen-binding fragment thereof according to claim 60.

70. A mouse B cell, wherein the genome of the B cell carries a sequence encoding a constant region, and the constant region sequence has S108P, F114A, L115A, R289K mutations and 327 K deletion mutations compared with the constant region of human IgG4.

71. A method of treating or preventing pain, cancer, inflammation or inflammatory diseases, neurodegenerative diseases, Sjogren's syndrome, endometriosis, diabetic peripheral neuropathy, prostatitis, pelvic pain syndrome, diseases related to imbalance in the regulation of bone remodeling and diseases caused by abnormal signaling of connective tissue growth factor in a subject, comprising administering to a subject in need thereof a therapeutically effective amount of the antibody or antigen-binding fragment thereof according to claim 60.

72. The method of treating or preventing neuropathic pain, inflammatory pain, cancer-related pain, fracture-related pain, surgery-related pain, inflammatory lung disease, interstitial cystitis, painful bladder syndrome, inflammatory bowel disease, inflammatory skin disease, Raynaud's syndrome, idiopathic pulmonary fibrosis, scar (hypertrophy, keloid type and other forms), sclerosis, endocardial myocardial fibrosis, atrial fibrosis, bone marrow fibrosis, progressive massive fibrosis (lung), renal-derived systemic fibrosis, scleroderma, systemic sclerosis, joint fibrosis, ocular fibrosis, non-small cell lung cancer, papillary thyroid cancer, glioblastoma multiforme, colorectal cancer, melanoma, bile duct cancer or sarcoma, acute myeloid leukemia, large cell neuroendocrine cancer, neuroblastoma, prostate cancer, pancreatic cancer, melanoma, head and neck squamous cell carcinoma or gastric cancer in a subject, comprising administering to a subject in need thereof a therapeutically effective amount of the antibody or antigen-binding fragment thereof according to claim 60.

73. A method of detecting TrkA or diagnosing a TrkA-related disease in a subject comprising administering to a subject in need thereof a therapeutically effective amount of the antibody or antigen-binding fragment thereof according to claim 60.

74. A method of detecting TrkA or diagnosing a TrkA-related disease comprising culturing the mouse B cell according to claim 70 so that the monoclonal antibody is produced.

75. A pharmaceutical composition comprising the nucleic acid molecule according to claim 67.

76. A method of treating or preventing pain, cancer, inflammation or inflammatory diseases, neurodegenerative diseases, Sjogren's syndrome, endometriosis, diabetic peripheral neuropathy, prostatitis, pelvic pain syndrome, diseases related to imbalance in the regulation of bone remodeling and diseases caused by abnormal signaling of connective tissue growth factor in a subject, comprising administering to a subject in need thereof a therapeutically effective amount of the nucleic acid molecule according to claim 67.

77. The method of treating or preventing neuropathic pain, inflammatory pain, cancer-related pain, fracture-related pain, surgery-related pain, inflammatory lung disease, interstitial cystitis, painful bladder syndrome, inflammatory bowel disease, inflammatory skin disease, Raynaud's syndrome, idiopathic pulmonary fibrosis, scar (hypertrophy, keloid type and other forms), sclerosis, endocardial myocardial fibrosis, atrial fibrosis, bone marrow fibrosis, progressive massive fibrosis (lung), renal-derived systemic fibrosis, scleroderma, systemic sclerosis, joint fibrosis, ocular fibrosis, non-small cell lung cancer, papillary thyroid cancer, glioblastoma multiforme, colorectal cancer, melanoma, bile duct cancer or sarcoma, acute myeloid leukemia, large cell neuroendocrine cancer, neuroblastoma, prostate cancer, pancreatic cancer, melanoma, head and neck squamous cell carcinoma or gastric cancer in a subject, comprising administering to a subject in need thereof a therapeutically effective amount of the antibody or antigen-binding fragment thereof according to claim 67.
